SEQUENCE LISTING (33 sequences)

SEQ ID NO: 1 - DNA, 97 bp, Artificial Sequence
  Description of Artificial Sequence: Synthetic oligonucleotide
acaagcggcacattgtgcctattgcgaattaggcacaatgtgcctaatctaacgtcatgccagccacaacggcgaggcgccaagaaggatagaagcc

SEQ ID NO: 2 - DNA, 35 bp, Artificial Sequence
  Description of Artificial Sequence: Synthetic oligonucleotide
tttacagctagctcagtcctagggactgtgctagc

SEQ ID NO: 3 - DNA, 155 bp, Artificial Sequence
  Description of Artificial Sequence: Synthetic polynucleotide
gatttttttcgggtgaggttgcgggctgttcggtaggtttataaacactgctatccaaagctatggacacgctcggctacgagaacagttggcgtgatggcctctagcaattagattgttatgcgacatccgcagacttggcagggagcgcacct

SEQ ID NO: 4 - DNA, 35 bp, Artificial Sequence
  Description of Artificial Sequence: Synthetic oligonucleotide
tttacggctagctcagtcctaggtatagtgctagc

SEQ ID NO: 5 - DNA, 199 bp, Artificial Sequence
  Description of Artificial Sequence: Synthetic polynucleotide
atccgagggatacgggccttgtcagcacggtgttgctaatgagagcctttgcccgggcaatagtacgggcagtgtgtagcggattgaaagacgctgaatcactgacaggcatgaagactatcgatagagtctgatagtgtcgccgccgcacagcggatagagtccacagtcattgaagtgttaatccgcgatcaagctc

SEQ ID NO: 6 - DNA, 216 bp, Artificial Sequence
  Description of Artificial Sequence: Synthetic polynucleotide
gacctagcttttatagcgggtttcgtggtttatagcccattgaaaaaaatctcacatctatatcacaggtgtgcactcgttcccgaaaggttctgagtctacttgatcaagtattgaaataccatcgtaaaggaaaaagacatgtctattcgtgatagcgaaaacaacaacggccaacagcagcagaccgcgcaaactgccgcccccgccccgcaa

SEQ ID NO: 7 - DNA, 182 bp, Artificial Sequence
  Description of Artificial Sequence: Synthetic polynucleotide
ttcaatttaagtagtaacgaggtcagcccggaatctttgggtattcttaaggtatttctgactcagtgtggttgggacagcttcactgtacattgcactggatttgttaatttcttataccggggcaccatgggcagcaaatcgtgttacgaattccgtctaaccaataagcgagctaaata

SEQ ID NO: 8 - DNA, 105 bp, Artificial Sequence
  Description of Artificial Sequence: Synthetic polynucleotide
cgcggaacccctatttgtttatttttctaaatacattcaaatatgtatccgctcatgagacaataaccctgataaatgcttcaataatattgaaaaaggaagagt

SEQ ID NO: 9 - DNA, 161 bp, Artificial Sequence
  Description of Artificial Sequence: Synthetic polynucleotide
cttcaagaattcgtattgaccccatagacagcttcgtcgacgcccgtcccggcccccttgggcttgccggacggcttatgtcatgatggcgccaccctcgcaggttcaaggccggctttcttcctctatgaacaaatcccttgcgctgactacgtaatcac

SEQ ID NO: 10 - DNA, 207 bp, Artificial Sequence
  Description of Artificial Sequence: Synthetic polynucleotide
cttcaagaattcggggtattcctgatcctgcgccgctagcgccgcgcacggccactaggcccgcgccgatagccagtcgcgctcccggctggcacactactcccatttccgccggaaacgcgcgcaacgtaccggcaacgaacgtggaaagaccatgaaagactggctggatgagattcactggaacgccgtgacctacgtatgcac

SEQ ID NO: 11 - DNA, 59 bp, Artificial Sequence
  Description of Artificial Sequence: Synthetic oligonucleotide
gaaaattattttaaatttcctctagtcaggccggaataactccctataatgcgacacca

SEQ ID NO: 12 - DNA, 34 bp, Artificial Sequence
  Description of Artificial Sequence: Synthetic oligonucleotide
gctcgactggtcggtaaccacttgtgtgtggtga

SEQ ID NO: 13 - DNA, 34 bp, Artificial Sequence
  Description of Artificial Sequence: Synthetic oligonucleotide
ggtgctgaccgaggacgagaaggaactgggcgtg

SEQ ID NO: 14 - DNA, 34 bp, Artificial Sequence
  Description of Artificial Sequence: Synthetic oligonucleotide
gatgacaccaacccggccaaggaagaccaggagt

SEQ ID NO: 15 - DNA, 34 bp, Artificial Sequence
  Description of Artificial Sequence: Synthetic oligonucleotide
atttatcacaaaaggattgttcgatgtccaacaa

SEQ ID NO: 16 - DNA, 34 bp, Artificial Sequence
  Description of Artificial Sequence: Synthetic oligonucleotide
cagtgcatggcagcgaacgccgagagccgacacc

SEQ ID NO: 17 - DNA, 34 bp, Artificial Sequence
  Description of Artificial Sequence: Synthetic oligonucleotide
tccgcgatgagctgccgtcccaacaattcaacac

SEQ ID NO: 18 - DNA, 34 bp, Artificial Sequence
  Description of Artificial Sequence: Synthetic oligonucleotide
aacgcgaagccctgttgaaaccgctgcaactggt

SEQ ID NO: 19 - DNA, 34 bp, Artificial Sequence
  Description of Artificial Sequence: Synthetic oligonucleotide
cgtaaacctaatgggcctgatctacagtaatcta

SEQ ID NO: 20 - DNA, 34 bp, Artificial Sequence
  Description of Artificial Sequence: Synthetic oligonucleotide
accaccgagacgcccacaccgtgcaagccgccgg

SEQ ID NO: 21 - DNA, 34 bp, Artificial Sequence
  Description of Artificial Sequence: Synthetic oligonucleotide
ctatcgcgaattcctgcaggctggcgcaaccaag

SEQ ID NO: 22 - skipped

SEQ ID NO: 23 - DNA, 6024 bp, Artificial Sequence
  Description of Artificial Sequence: Synthetic polynucleotide
acaagcggcacattgtgcctattgcgaattaggcacaatgtgcctaatctaacgtcatgccagccacaacggcgaggcgccaagaaggatagaagccgtcgcgccccgcacgggcgcgtggattgaaacggtgctgaccgaggacgagaaggaactgggcgtggtcgcgccccgcacgggcgcgtggattgaaactccgcgatgagctgccgtcccaacaattcaacacgtcgcgccccgcacgggcgcgtggattgaaacaccaccgagacgcccacaccgtgcaagccgccgggtcgcgccccgcacgggcgcgtggattgaaaccatgcaagcttggcgtaggccgcttcgtccctatcaaagcttggagtttacagctagctcagtcctagggactgtgctagcattaaagaggagaaaatggacgcggaggctagcgatactcacttttttgctcactccaccttaaaggcagatcgcagcgattggcagcctctggtcgagcatctacaggctgttgcccgtttggcaggagagaaggctgccttcttcggcggcggtgaattagctgctcttgctggtctgttgcatgacttgggtaaatacactgacgagtttcagcggcgtattgcgggtgatgccatccgtgtcgatcactctactcgcggggccatactggcggtagaacgctatggcgcgctaggtcaattgctagcctacggcatcgctggccaccatgccgggttggccaatggccgcgaggctggtgagcgaactgccttggtcgaccgcctgaaaggggttgggctgccacggttattggaggggtggtgcgtggaaatcgtgctacccgagcgccttcaaccaccgccactaaaagcgcgcctggaaagaggtttctttcagttggcctttcttggccggatgctcttttcctgcttggttgatgcggattatctagataccgaagccttctaccaccgcgtcgaaggacggcgctcccttcgcgagcaagcgcggccgaccttggccgagttacgcgcagcccttgatcggcatctgactgagttcaagggagatacgccggtcaaccgcgttcgcggggagatattggccggcgtgcgcggcaaggcgagcgaacttcccgggctgttttctctcacagtgcccacaggaggcggcaagaccctggcctctctggctttcgccctggatcacgctctagctcatgggctgcgccgggtgatctacgtgattcccttcactagcatcgtcgagcagaacgctgcggtattccgtcgtgcactcggggccttaggcgaagaggcggtgctggagcatcacagcgccttcgttgatgaccgccggcagagcctggaggccaagaagaaactgaacctagcgatggagaactgggacgcgcctatcgtggtgaccactgcagtgcagttcttcgaaagcctgtttgccgaccgtccagcccagtgccgcaagctacacaacatcgccggcagcgtggtgattcttgacgaggcacagaccctaccgctcaagctgttgcggccctgcgttgccgcccttgatgaactggcgctcaactaccgttgtagcccagttctctgtactgccacgcagccagcgcttcaatcgccggatttcatcggtgggctgcaggacgtacgtgagctggcgcccgagccgcagcggctgttccgggagttggtgcgggtacgaatacggacattgggcccgctcgaagatgcggccttgactgagcagatcgccaggcgtgaacaagtgctgtgcatcgtcaacaatcgacgccaggcccgtgcgctctatgagtcgcttgccgagttgcccggtgcccgccatctcaccaccctgatgtgcgccaagcaccgtagcagcgtgctggccgaggtgcgccagatgctcaaaaagggggagccctgtcgcctggtggccacctcgctgatcgaggccggtgtggatgtggattttcccgtggtactgcgtgccgaggctggattggattccatcgcccaggccgcgggacgctgcaatcgcgaaggcaagcggccgctggccgaaagcgaggtgctggtgttcgccgcggccaattctgactgggcgccacccgaggaactcaagcagttcgcccaggccgcccgcgaagtgatgcgcctgcacccggatgattgcctgtccatggcggccatcgagcggtattttcgcatactgtactggcagaagggcgcggaggagttggatgcgggtaacctgctcggcctgattgagagaggccggctcgatggcctgccctacgagactttggccaccaagttccgcatgatcgacagccttcaactgccggtgatcatcccatttgatgacgaggccagagcagccctgcgcgagctggagttcgccgacggctgcgccgccatcgcccgtcgcctgcagccatatctggtgcagatgccacgcaagggttatcaggcattgcgggaagccggtgcgatccaggcggcggcaggtacgcgttatggtgagcagtttatggcgttggtcaaccctgatctgtatcaccaccaattcgggttgcactgggataatccggcctttgtcagcagcgagcggctatgttggtagtcgggacgcgcaacagcggcctggcctggatgatgtgaaagggagggccgatggcctacggaattcgcttaatggtctggggcgagcgtgcctgcttcacccgcccggaaatgaaggtggaacgcgtctcttacgatgcgatcacgccgtccgccgcgcgcggcattctcgaggctatccactggaagccggcgattcgctgggtggtggatcgcattcaagtgcttaagccgatccgcttcgaatccatccggcgcaacgaggtcggcggcaagctgtccgctgtcagcgtcggtaaggcaatgaaggccgggcgtactaatggtctggtgaatctggtcgaggaggatcgccagcagcgcgcgactactctgctgcgcgatgtctcctatgtcatcgaggcgcatttcgagatgactgacagggctggcgccgacgatacggtgggcaagcatctggatatcttcaaccgtcgcgcacggaaggggcagtgcttccatacaccctgcctaggcgtgcgcgagtttccggccagttttcggttgctggaagagggcagtgccgagcctgaagtcgatgcctttctgcgcggcgagcgtgatctgggctggatgctgcatgacattgacttcgccgatggcatgaccccgcacttcttccgtgccctgatgcgcgatgggctgatcgaggtgccggccttcagggcggcagaggacaaggcatgatcctttcggccctcaatgactattatcagcgactgctggagcggggtgaagcgaatatctcacccttcggctacagccaagaaaagatcagttacgccctgctgctgtccgcacaaggagagttgctggacgtgcaggacattcgcttgctctctggcaagaagcctcaacccaggcttatgagtgtgccgcagccggagaagcgcacctcgggcatcaagtccaacgtactgtgggacaagaccagctatgtgctgggtgttagtgccaagggcggagagcgtactcagcaggagcacgagtccttcaagacgctgcaccggcagatcttggttggggaaggcgaccccggtctgcaggccttgctccagttcctcgactgttggcagccggagcagttcaagcccccgctgttcagcgaagcaatgctcgacagcaacttagtgttccgcctagacggccaacaacgctatctgcacgagactccggcggccctggcgttgcgtacccggctgttggccgacggcgacagccgcgaggggctgtgcctagtctgcggccaacgtcagccgttggcgcgcctgcatccagcggtcaagggcgtcaatggtgcccagagttcgggggcttccatcgtctccttcaacctcgacgctttttcctcctacggcaagagccagggggaaaatgctccggtctccgaacaggccgcctttgcctacaccacggtgctcaaccatttgttgcgtcgcgacgagcacaaccgccagcgcctgcagattggcgacgcgagtgtggtgttctgggcgcaggcggatactcctgctcaggtggccgccgccgagtcgaccttctggaacctgctggagccacccgcagatgatggtcaggaagcggaaaagctgcgcggcgtgctggatgctgtggccacggggcggcccttgcatgagctcgactcgctaatggaggaaggtacccgcatttttgtgttagggctggcgcccaatacctcgcgactgtccattcggttctgggcagtcgatagccttgcggtattcacccagcatctggccgagcatttccgggatatgcaccttgagcctctgccctggaagacggagccggccatctggcgcttgctctatgctaccgcgcccagtcgtgacggcagagccaagaccgaagacgtactcccacaactggccggtgaaatgacccgcgccatcctgaccggcagccgctatccgcgcagtttgctagccaacctgatcatgcgcatgcgtgccgacggcgacgtctctggcatacgcgtcgcgctgtgcaaggccgtgctcgctcgcgaggcacgcctgagcggcaaaattcaccaagaggagctacctatgagtctcgacaaggacgccagcaaccccggctatcgcttggggaggctgttcgccgtgttggaaggcgcccagcgcgcagccctgggcgacagggtcaatgccactatccgtgaccgctactacggtgccgcgtccagcacgccagccacggttttcccgatactgctgcgcaacacacaaaaccacttggccaagctgcgcaaggagaagcccggactagcagtgaacctagagcgcgatataggcgaaatcattgacggtatgcagagccaattcccgcgttgcctgcgcctggaggaccagggacgctttgctattggttactaccaacaggcccaggcccgtttcaaccgtggccccgattccgtcgagtaaggagcagaagaatgaccgccatctccaaccgctacgagttcgtttacctctttgatgtcagcaatggcaatcccaatggcgacccggatgctggcaacatgccgcgtctcgatccggaaaccaaccaggggttggtcactgacgtttgcctcaagcgcaagatccgcaactacgtcagcctggagcaggaaagtgcccccggctatgccatctatatgcaggaaaaatccgtgctgaataaccagcacaaacaggcctacgaggcgctcggtatcgagtcagaggcaaagaaactgcccaaggacgaagccaaggcgcgcgaactgacctcttggatgtgcaagaacttcttcgatgtgcgtgctttcggggcggtgatgaccaccgagattaatgccggccaggtgcgtggaccgatccaactggcattcgccacgtctatcgacccggtattgcctatggaggtatccatcacccgcatggcggtgactaacgaaaaggatttggagaaggaacgcaccatgggacgcaagcacatcgtgccttacggcttgtaccgcgcccatggtttcatctctgccaagttggccgagcgaaccggcttttccgacgacgacttggaactgctatggcgcgctttggccaatatgttcgaacacgaccgctcggcggcacgtggcgagatggcagcgcgcaagttgatcgtcttcaagcatgagcatgccatgggcaatgcacccgcccatgtgctgttcggcagcgttaaggtcgagcgagtcgagggggacgcagttacaccagcacgcggtttccaggattaccgtgtcagcatcgatgcggaagctctgcctcagggcgtgagcgtgcgcgagtacctctag

SEQ ID NO: 24 - DNA, 32 bp, Artificial Sequence
  Description of Artificial Sequence: Synthetic oligonucleotide
gtcgcgccccgcacgggcgcgtggattgaaac

SEQ ID NO: 25 - DNA, 32 bp, Artificial Sequence
  Description of Artificial Sequence: Synthetic oligonucleotide
gtcgcgccccgcacgggcgcgtggagtgaaag

SEQ ID NO: 26 - DNA, 32 bp, Artificial Sequence
  Description of Artificial Sequence: Synthetic oligonucleotide
gtcgcgccccgcacgggtgcgtggattgaaac

SEQ ID NO: 27 - DNA, 32 bp, Artificial Sequence
  Description of Artificial Sequence: Synthetic oligonucleotide
gtcgcgccccgcatgggcgcgtggattgaaca

SEQ ID NO: 28 - DNA, 32 bp, Artificial Sequence
  Description of Artificial Sequence: Synthetic oligonucleotide
gtcgcgccctacgcgggcgcgtggagtgaaag

SEQ ID NO: 29 - DNA, 373 bp, Artificial Sequence
  Description of Artificial Sequence: Synthetic polynucleotide
acaagcggcacattgtgcctattgcgaattaggcacaatgtgcctaatctaacgtcatgccagccacaacggcgaggcgccaagaaggatagaagccgtcgcgccccgcacgggcgcgtggattgaaacgctcgactggtcggtaaccacttgtgtgtggtgagtcgcgccccgcacgggcgcgtggattgaaaccagtgcatggcagcgaacgccgagagccgacaccgtcgcgccccgcacgggcgcgtggattgaaaccgtaaacctaatgggcctgatctacagtaatctagtcgcgccccgcacgggcgcgtggattgaaaccatgcaagcttggcgtaggccgcttcgtccctatcaaagcttggag

SEQ ID NO: 30 - DNA, 373 bp, Artificial Sequence
  Description of Artificial Sequence: Synthetic polynucleotide
acaagcggcacattgtgcctattgcgaattaggcacaatgtgcctaatctaacgtcatgccagccacaacggcgaggcgccaagaaggatagaagccgtcgcgccccgcacgggcgcgtggattgaaacggtgctgaccgaggacgagaaggaactgggcgtggtcgcgccccgcacgggcgcgtggattgaaactccgcgatgagctgccgtcccaacaattcaacacgtcgcgccccgcacgggcgcgtggattgaaacaccaccgagacgcccacaccgtgcaagccgccgggtcgcgccccgcacgggcgcgtggattgaaaccatgcaagcttggcgtaggccgcttcgtccctatcaaagcttggag

SEQ ID NO: 31 - DNA, 373 bp, Artificial Sequence
  Description of Artificial Sequence: Synthetic polynucleotide
acaagcggcacattgtgcctattgcgaattaggcacaatgtgcctaatctaacgtcatgccagccacaacggcgaggcgccaagaaggatagaagccgtcgcgccccgcacgggcgcgtggattgaaacgatgacaccaacccggccaaggaagaccaggagtgtcgcgccccgcacgggcgcgtggattgaaacaacgcgaagccctgttgaaaccgctgcaactggtgtcgcgccccgcacgggcgcgtggattgaaacctatcgcgaattcctgcaggctggcgcaaccaaggtcgcgccccgcacgggcgcgtggattgaaaccatgcaagcttggcgtaggccgcttcgtccctatcaaagcttggag

SEQ ID NO: 32 - DNA, 177 bp, Artificial Sequence
  Description of Artificial Sequence: Synthetic polynucleotide
gaaaattattttaaatttcctctagtcaggccggaataactccctataatgcgacaccagtcgcgccccgcacgggcgcgtggattgaaacatttatcacaaaaggattgttcgatgtccaacaagtcgcgccccgcacgggcgcgtggattgaaacgcactcccgttctggataat

SEQ ID NO: 33 - DNA, 6024 bp, Artificial Sequence; identical to SEQ ID NO: 23
  Description of Artificial Sequence: Synthetic polynucleotide

(19) United States
(12) Patent Application Publication
(10) Pub. No.: US 2022/0370526 A1
(43) Pub. Date: Nov. 24, 2022

(54) PHAGE COMPOSITIONS COMPRISING CRISPR-CAS SYSTEMS AND METHODS OF USE THEREOF

(71) Applicant: Locus Biosciences, Inc., Morrisville, NC (US)

(72) Inventors: David G. OUSTEROUT, Morrisville, NC (US); Paul M. GAROFOLO, Morrisville, NC (US); Kurt SELLE, Morrisville, NC (US); Hannah Hewitt TUSON, Morrisville, NC (US)

(21) Appl. No.: 17/774,393

(22) PCT Filed: Nov. 5, 2020
(86) PCT No.: PCT/US2020/059159

§ 371 Date: May 4, 2022

Related U.S. Application Data
(60) Provisional application No. 62/931,795, filed on Nov. 6, 2019.
(60) Provisional application No. 63/088,394, filed on Oct. 6, 2020.

(51) Int. Cl. A61K 35/76; C12N 15/86; C12N 9/22; C12N 15/11; A61P 31/04

(52) U.S. Cl. CPC A61K 35/76; C12N 15/86; C12N 9/22; C12N 15/11; A61P 31/04; C12N 2800/80; C12N 2310/20; C12N 2795/00032; C12N 2795/00042; C12N 2795/00022; A61K 38/00

(57) ABSTRACT

Disclosed here are phage compositions comprising CRISPR-Cas systems and methods of use thereof. In certain embodiments, disclosed herein is a bacteriophage comprising a nucleic acid sequence encoding a Type I CRISPR-Cas system comprising: (a) a CRISPR array; (b) a Cascade polypeptide; and (c) a Cas3 polypeptide. In some embodiments, the CRISPR array comprises a spacer sequence and at least one repeat sequence. In some embodiments, the at least one repeat sequence is operably linked to the spacer sequence at either its 5′ end or its 3′ end.

### CROSS-REFERENCE

This application claims the benefit of U.S. Patent Application No. 62/931,795, filed Nov. 6, 2019, and U.S. Patent Application No. 63/088,394 filed Oct. 6, 2020, which are hereby incorporated by reference in their entirety.

### SUMMARY

Disclosed herein, in certain embodiments, are phage compositions comprising CRISPR-Cas systems and methods of use thereof.

In certain embodiments, disclosed herein is a bacteriophage comprising a nucleic acid sequence encoding a Type I CRISPR-Cas system comprising: (a) a CRISPR array; (b) a Cascade polypeptide; and (c) a Cas3 polypeptide. In some embodiments, the CRISPR array comprises a spacer sequence and at least one repeat sequence. In some embodiments, the at least one repeat sequence is operably linked to the spacer sequence at either its 5′ end or its 3′ end. In some embodiments, the spacer sequence is complementary to a target nucleotide sequence in a target bacterium. In some embodiments, the target nucleotide sequence comprises a coding sequence. In some embodiments, the target nucleotide sequence comprises a non-coding or intergenic sequence. In some embodiments, the target nucleotide sequence comprises all or a part of a promoter sequence. In some embodiments, the target nucleotide sequence comprises all or a part of a nucleotide sequence located on a coding strand of a transcribed region of an essential gene. In some embodiments, the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK. In some embodiments, the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system. In some embodiments, the Cascade complex comprises: (i) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (ii) a Cas6b polypeptide, a Cas8b polypeptide, a Cas7 polypeptide, and a Cas5 polypeptide (Type I-B CRISPR-Cas system); (iii) a Cas5d polypeptide, a Cas8c polypeptide, and a Cas7 polypeptide (Type I-C CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); (vi) a Csy1 polypeptide, a Csy2 polypeptide, a Csy3 polypeptide, and a Csy4 polypeptide (Type I-F CRISPR-Cas system). In some embodiments, the Cas complex comprises a Cas5d polypeptide, a Cas8c polypeptide, and a Cas7 polypeptide (Type I-C CRISPR-Cas system). In some embodiments, the nucleic acid sequence further comprises a promoter sequence. In some embodiments, the target bacterium is killed solely by lytic activity of the bacteriophage. In some embodiments, the target bacterium is killed solely by activity of the CRISPR-Cas system. In some embodiments, the target bacterium is killed by lytic activity of the bacteriophage in combination with activity of the CRISPR-Cas system. In some embodiments, the target bacterium is killed by the activity of the CRISPR-Cas system, independently of the lytic activity of the bacteriophage. In some embodiments, the activity of the CRISPR-Cas system supplements or enhances the lytic activity of the bacteriophage. In some embodiments, the lytic activity of the bacteriophage and the activity of the CRISPR-Cas system are synergistic. In some embodiments, the lytic activity of the bacteriophage, the activity of the CRISPR-Cas system, or both, is modulated by a concentration of the bacteriophage In some embodiments, the bacteriophage infects multiple bacterial strains. In some embodiments, the target bacterium is an Acinetobacter species, an Actinomyces species, Burkholderia cepacia complex, a Campylobacter species, a Candida species, Clostridium difficile, Corynebacterium minutissium, Corynebacterium pseudodiphtheriae, Corynebacterium stratium, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter felis, Helicobacter pylori, Clostridium bolteae, and any combination thereof. In some embodiments, the bacteriophage is an obligate lytic bacteriophage. In some embodiments, the bacteriophage is a temperate bacteriophage that is rendered lytic. In some embodiments, the temperate bacteriophage is rendered lytic by the removal, replacement, or inactivation of a lysogeny gene. In some embodiments, the bacteriophage is p1772, p2131, p2132, p2973, p4209, p1106, p1587, p1835, p2037, p2421, p2363, p004k, or PB1. In some embodiments, the nucleic acid sequence is inserted into a non-essential bacteriophage gene. In some embodiments, described herein is a pharmaceutical composition comprising: (a) the bacteriophage described herein; and (b) a pharmaceutically acceptable excipient. In some embodiments, the pharmaceutical composition is in the form of a tablet, a liquid, a syrup, an oral formulation, an intravenous formulation, an intranasal formulation, an ocular formulation, an otic formulation, a subcutaneous formulation, an inhalable respiratory formulation, a suppository, and any combination thereof.

In some embodiments, disclosed herein is a method of killing a target bacterium comprising introducing into the target bacterium a bacteriophage comprising a nucleic acid sequence encoding a Type I CRISPR-Cas system comprising: (a) a CRISPR array; (b) a Cascade polypeptide; and (c) a Cas3 polypeptide. In some embodiments, the CRISPR array comprises a spacer sequence and at least one repeat sequence. In some embodiments, the at least one repeat sequence is operably linked to the spacer sequence at either its 5′ end or its 3′ end. In some embodiments, the spacer sequence is complementary to a target nucleotide sequence in a target bacterium. In some embodiments, the target nucleotide sequence comprises a coding sequence. In some embodiments, the target nucleotide sequence comprises a non-coding or intergenic sequence. In some embodiments, the target nucleotide sequence comprises all or a part of a promoter sequence. In some embodiments, the target nucleotide sequence comprises all or a part of a nucleotide sequence located on a coding strand of a transcribed region of an essential gene. In some embodiments, the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK. In some embodiments, the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system. In some embodiments, the Cascade complex comprises: (i) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (ii) a Cas6b polypeptide, a Cas8b polypeptide, a Cas7 polypeptide, and a Cas5 polypeptide (Type I-B CRISPR-Cas system); (iii) a Cas5d polypeptide, a Cas8c polypeptide, and a Cas7 polypeptide (Type I-C CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); (vi) a Csy1 polypeptide, a Csy2 polypeptide, a Csy3 polypeptide, and a Csy4 polypeptide (Type I-F CRISPR-Cas system). In some embodiments, the Cascade complex comprises a Cas5d polypeptide, a Cas8c polypeptide, and a Cas7 polypeptide (Type I-C CRISPR-Cas system). In some embodiments, the nucleic acid sequence further comprises a promoter sequence. In some embodiments, the target bacterium is killed solely by activity of the CRISPR-Cas system. In some embodiments, the target bacterium is killed by lytic activity of the bacteriophage in combination with activity of the CRISPR-Cas system. In some embodiments, the target bacterium is killed by the activity of the CRISPR-Cas system, independently of the lytic activity of the bacteriophage. In some embodiments, the activity of the CRISPR-Cas system supplements or enhances the lytic activity of the bacteriophage. In some embodiments, the lytic activity of the bacteriophage and the activity of the CRISPR-Cas system are synergistic. In some embodiments, the lytic activity of the bacteriophage, the activity of the CRISPR-Cas system, or both, is modulated by a concentration of the bacteriophage In some embodiments, the bacteriophage infects multiple bacterial strains. In some embodiments, the target bacterium is an Acinetobacter species, an Actinomyces species, Burkholderia cepacia complex, a Campylobacter species, a Candida species, Clostridium difficile, Corynebacterium minutissium, Corynebacterium pseudodiphtheriae, Corynebacterium stratium, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter felis, Helicobacter pylori, Clostridium bolteae, and any combination thereof. In some embodiments, the bacteriophage is an obligate lytic bacteriophage. In some embodiments, the bacteriophage is a temperate bacteriophage that is rendered lytic. In some embodiments, the temperate bacteriophage is rendered lytic by the removal, replacement, or inactivation of a lysogeny gene. In some embodiments, the bacteriophage is p1772, p2131, p2132, p2973, p4209, p1106, p1587, p1835, p2037, p2421, p2363, p4209, p004k, or PB1. In some embodiments, the nucleic acid sequence is inserted in pace of or adjacent to a non-essential bacteriophage gene. In some embodiments, a mixed population of bacterial cells comprises the target bacterium.

In some embodiments, disclosed herein is a method of treating a disease in an individual in need thereof, the method comprising administering to the individual a bacteriophage comprising a nucleic acid sequence encoding a Type I CRISPR-Cas system comprising: (a) a CRISPR array; (b) a Cascade polypeptide; and (c) a Cas3 polypeptide. In some embodiments, the CRISPR array comprises a spacer sequence and at least one repeat sequence. In some embodiments, the at least one repeat sequence is operably linked to the spacer sequence at either its 5′ end or its 3′ end. In some embodiments, the spacer sequence is complementary to a target nucleotide sequence in a target bacterium. In some embodiments, the target nucleotide sequence comprises a coding sequence. In some embodiments, the target nucleotide sequence comprises a non-coding or intergenic sequence. In some embodiments, the target nucleotide sequence comprises all or a part of a promoter sequence. In some embodiments, the target nucleotide sequence comprises all or a part of a nucleotide sequence located on a coding strand of a transcribed region of an essential gene. In some embodiments, the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK. In some embodiments, the Cascade complex comprises Cascade polypeptides of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system. In some embodiments, the Cascade complex comprises: (i) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (ii) a Cas6b polypeptide, a Cas8b polypeptide, a Cas7 polypeptide, and a Cas5 polypeptide (Type I-B CRISPR-Cas system); (iii) a Cas5d polypeptide, a Cas8c polypeptide, and a Cas7 polypeptide (Type I-C CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); (vi) a Csy1 polypeptide, a Csy2 polypeptide, a Csy3 polypeptide, and a Csy4 polypeptide (Type I-F CRISPR-Cas system). In some embodiments, the CASCADE complex comprises a Cas5d polypeptide, a Cas8c polypeptide, and a Cas7 polypeptide (Type I-C CRISPR-Cas system). In some embodiments, the nucleic acid sequence further comprises a promoter sequence. In some embodiments, the target bacterium is killed solely by activity of the CRISPR-Cas system. In some embodiments, the target bacterium is killed by lytic activity of the bacteriophage in combination with activity of the CRISPR-Cas system. In some embodiments, the target bacterium is killed by the activity of the CRISPR-Cas system, independently of the lytic activity of the bacteriophage. In some embodiments, the activity of the CRISPR-Cas system supplements or enhances the lytic activity of the bacteriophage. In some embodiments, the lytic activity of the bacteriophage and the activity of the CRISPR-Cas system are synergistic. In some embodiments, the lytic activity of the bacteriophage, the activity of the CRISPR-Cas system, or both, is modulated by a concentration of the bacteriophage In some embodiments, the bacteriophage infects multiple bacterial strains. In some embodiments, the bacteriophage is an obligate lytic bacteriophage. In some embodiments, the bacteriophage is a temperate bacteriophage that is rendered lytic. In some embodiments, the temperate bacteriophage is rendered lytic by the removal, replacement, or inactivation of a lysogeny gene. In some embodiments, the bacteriophage is p1772, p2131, p2132, p2973, p4209, p1106, p1587, p1835, p2037, p2421, p2363, p4209, p004k, or PB1. In some embodiments, the nucleic acid sequence is inserted in pace of or adjacent to a non-essential bacteriophage gene. In some embodiments, the disease is a bacterial infection. In some embodiments, the target bacterium causing the disease is a drug resistant bacterium that is resistant to at least one antibiotic. In some embodiments, the drug resistant bacterium is resistant to at least one antibiotic. In some embodiments, the target bacterium causing the disease is a multidrug resistant bacterium. In some embodiments, the multi-drug resistant bacterium is resistant to at least one antibiotic. In some embodiments, the antibiotic comprises a cephalosporin, a fluoroquinolone, a carbapenem, a colistin, an aminoglycoside, vancomycin, streptomycin, or methicillin. In some embodiments, the target bacterium causing the bacterial infection is an Acinetobacter species, an Actinomyces species, Burkholderia cepacia complex, a Campylobacter species, a Candida species, Clostridium difficile, Corynebacterium minutissium, Corynebacterium pseudodiphtheriae, Corynebacterium stratium, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter felis, Helicobacter pylori, Clostridium bolteae, and any combination thereof. In some embodiments, the target bacterium causing the disease is Pseudomonas. In some embodiments, the target bacterium causing the disease is P. aeruginosa. In some embodiments, the administering is intra-arterial, intravenous, intraurethral, intramuscular, oral, subcutaneous, inhalation, or any combination thereof. In some embodiments, the individual is a mammal.

In some embodiments, disclosed herein is a bacteriophage comprising a nucleic acid sequence encoding a Type I CRISPR-Cas system comprising: a CRISPR array; a Cascade polypeptide comprising Cas5, Cas8c and Cas7; and a Cas3 polypeptide. In some embodiments, the CRISPR array comprises a spacer sequence and at least one repeat sequence. In some embodiments, the at least one repeat sequence is operably linked to the spacer sequence at either its 5′ end or its 3′ end. In some embodiments, the spacer sequence is complementary to a target nucleotide sequence in a target bacterium. In some embodiments, the target nucleotide sequence comprises a coding sequence. In some embodiments, the target nucleotide sequence comprises a non-coding or intergenic sequence. In some embodiments, the target nucleotide sequence comprises all or a part of a promoter sequence. In some embodiments, the target nucleotide sequence comprises all or a part of a nucleotide sequence located on a coding strand of a transcribed region of an essential gene. In some embodiments, the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK. In some embodiments, the nucleic acid sequence further comprises a promoter sequence. In some embodiments, the target bacterium is killed solely by lytic activity of the bacteriophage. In some embodiments, the target bacterium is killed solely by activity of the CRISPR-Cas system. In some embodiments, the target bacterium is killed by lytic activity of the bacteriophage in combination with activity of the CRISPR-Cas system. In some embodiments, the target bacterium is killed by the activity of the CRISPR-Cas system, independently of the lytic activity of the bacteriophage. In some embodiments, the activity of the CRISPR-Cas system supplements or enhances the lytic activity of the bacteriophage. In some embodiments, the lytic activity of the bacteriophage and the activity of the CRISPR-Cas system are synergistic. In some embodiments, the lytic activity of the bacteriophage, the activity of the CRISPR-Cas system, or both, is modulated by a concentration of the bacteriophage In some embodiments, the bacteriophage infects multiple bacterial strains. In some embodiments, the target bacterium is an Acinetobacter species, an Actinomyces species, Burkholderia cepacia complex, a Campylobacter species, a Candida species, Clostridium difficile, Corynebacterium minutissium, Corynebacterium pseudodiphtheriae, Corynebacterium stratium, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter felis, Helicobacter pylori, Clostridium bolteae, and any combination thereof. In some embodiments, the bacteriophage is an obligate lytic bacteriophage. In some embodiments, the bacteriophage is a temperate bacteriophage that is rendered lytic. In some embodiments, the temperate bacteriophage is rendered lytic by the removal, replacement, or inactivation of a lysogeny gene. In some embodiments, the bacteriophage is p1772, p2131, p2132, p2973, p4209, p1106, p1587, p1835, p2037, p2421, p2363, p4209, p004k, or PB1. In some embodiments, the nucleic acid sequence is inserted into a non-essential bacteriophage gene. In some embodiments, disclosed herein is a pharmaceutical composition comprising: (a) the bacteriophage disclosed herein; and (b) a pharmaceutically acceptable excipient. In some embodiments, the pharmaceutical composition is in the form of a tablet, a liquid, a syrup, an oral formulation, an intravenous formulation, an intranasal formulation, an ocular formulation, an otic formulation, a subcutaneous formulation, an inhalable respiratory formulation, a suppository, and any combination thereof.

In some embodiments, disclosed herein is a method of sanitizing a surface in need thereof, the method comprising administering to the surface a bacteriophage comprising a nucleic acid sequence encoding a Type I CRISPR-Cas system comprising: (d) a CRISPR array; (e) a Cascade polypeptide; and (f) a Cas3 polypeptide. In some embodiments, the surface is a hospital surface, a vehicle surface, an equipment surface, or an industrial surface.

In some embodiments, disclosed herein is a method of preventing contamination in a food product or a nutritional supplement, the method comprising administering to the a food product or the nutritional supplement a bacteriophage comprising a nucleic acid sequence encoding a Type I CRISPR-Cas system comprising: (a) a CRISPR array; (b) a Cascade polypeptide; and (c) a Cas3 polypeptide. In some embodiments, the food product or nutritional supplement comprises milk, yoghurt, curd, cheese, fermented milks, milk based fermented products, ice-creams, fermented cereal based products, milk based powders, infant formulae or tablets, liquid suspensions, dried oral supplement, wet oral supplement, or dry-tube-feeding.

### BRIEF DESCRIPTION OF THE DRAWINGS

The novel features of the disclosures are set forth with particularity in the appended claims. A better understanding of the features and advantages of the present disclosures will be obtained by reference to the following detailed description that sets forth illustrative embodiments, in which the principles of the disclosures are utilized, and the accompanying drawings of which:

FIG. 1A depicts the sequence and arrangement of crArray2. FIG. 1B depicts the sequence and arrangement of crArray3. FIG. 1C depicts the sequence and arrangement of crArray3. FIG. 1D depicts the sequence and arrangement of crArray4. FIG. 1E depicts the sequence and arrangement of crArray5.

FIG. 2A (top panel) depicts the effects of transforming two P. aeruginosa strains with a plasmid containing crRNA using endogenous CRISPR-Cas3 system as measured by the number of transformants in colony forming units (CFU). The bottom panel shows the effects of transforming P. aeruginosa with a plasmid containing both a crRNA containing 3 spacers and an exogenous Type I-C Cas operon, which results in fewer transformants than the limit of detection. FIG. 2B depicts the number of bacterial transformants obtained per mL of transformation into a Cas operon null mutant of P. aeruginosa strain b1121. Array 1 targets the bacterial genome while array 2 is a non-targeting control. The different plasmids were normalized by molarity to the empty vector control plasmid. FIG. 2C depicts the effects of transforming individual spacers targeting rpoB or ftsA or 3-spacer arrays Array 3 or Array 4 into P. aeruginosa strains with (b1121) or without (b1121 cas KO) an endogenous Type I-C Cas operon.

FIG. 3A depicts a schematic representation of the genome of wild type phage p1772 and its engineered variants. The bar below the genome axis indicates the region of the genome that was removed and replaced. The schematics below the phage genome illustrate the DNA that was used to replace WT phage genes in the deleted region. Array 1, Array 3, and Array 4 target the bacterial genome and will kill bacteria in the presence of a Type I-C Cas operon. The spacers in Array 2 are non-targeting, but the array is structurally the same as the three targeting arrays. FIG. 3B compares the sequences of p1772e005 (targeting crArray1+Cas system) after it had been passaged 5 or 10 times at 37 degrees Celsius. No difference was observed in the insert at the nucleotide level indicating the stability of engineered phages expressing CRISPR-Cas3.

FIG. 4 exemplifies that phage engineered with CRISPR-Cas3 does not exhibit structural changes. There were no gross morphological differences between p1772 wt (wild type), p1772e004 (Cas system only), and p1772e005 (targeting crArray+Cas system) when imaged by TEM.

FIG. 5A-FIG. 5C exemplify full construct phage amplifies similarly to the wild type parent phage in variants of different Cas types and retains a similar host range. FIG. 5A depict the in vitro amplification titers of p1772 wt (wild type), p1772e004 (Cas system), and p1772e005 (targeting crArray1+Cas system) in P. aeruginosa strains containing Type I-F Cas systems. FIG. 5B depicts the in vitro amplification titers of p1772 wt and p1772e005 in P. aeruginosa strains containing Type I-C Cas systems. FIG. 5C depicts the host range of p1772 wt, p1772e004 and p1772e005 on 44 strains of P. aeruginosa. The phage is considered to infect a given strain if (AUC in the presence of phage)/(AUC in the absence of phage) is less than 0.65.

FIG. 6A-FIG. 6E exemplify exogenous CRISPR-Cas3 system is efficiently expressed from the phage genome. FIG. 6A depicts a schematic of the spacer array and Cas operon inserted into engineered variants of p1772. FIG. 6B-6D depict the expression of the crArray, Cas3 and Cas8 in P. aeruginosa strain b1121 infected with p1772 wt (wild type) or p1772e005 (targeting crArray1+Cas system) over 1600 minutes. FIG. 6E depicts the expression of Cas3 1 and 24 hours after infection with p1772e005, p2131e002 (targeting crArray1+Cas system) and p2132e002 (targeting crArray1+Cas system).

FIG. 7 depicts plaques resulting from plating p1772 wt (wild type) or p1772e005 (targeting crArray1+Cas system) onto P. aeruginosa.

FIG. 8A exemplifies the results of a plate-based kill assay. p1772 wt (wild type), p1772e004 (Cas system only), p1772e006 (targeting crArray1 only) and p1772e005 (targeting crArray1+Cas system) were mixed with P. aeruginosa at multiplicities of infection (MOIs) from 100 to 0.0000954. FIG. 8B depicts a portion of a plate set up as in FIG. 8A at greater magnification. FIG. 8C shows a quantification of the relative fluorescent units of P. aeruginosa infected with p1772 wt, p1772e008 (non-targeting crArray2+Cas system), p1772e006 and p1772e005 at a MOI of 1.5

FIG. 9A depicts the growth of p1772 wt (wild type), p1772e004 (Cas system only), p1772e005 (targeting crArray1+Cas system) and p1772e006 (targeting crArray1 only) in the P. aeruginosa strain b1121 over 24 hours when inoculated at an MOI of 1. FIG. 9B depicts the growth of p1772 wt, p1772e004, p1772e005 and p1772e006 in the P. aeruginosa strain b1121 over 24 hours when inoculated at an MOI of 10. FIG. 9C depicts the growth of p1772 wt, p1772e004, p1772e005 and p1772e006 in the P. aeruginosa strain b1121 over 24 hours when inoculated at an MOI of 100.

FIG. 10A depicts the growth on an agar plate of P. aeruginosa cultures mixed with p1772 wt (wild type), p1772e008 (non-targeting crArray2+Cas system), p1772e006 (targeting crArray 1 only), and pArray3 (targeting crArray3+Cas system) at MOIs from 100 to 0.0001. FIG. 10B depicts the growth on an agar plate of P. aeruginosa cultures mixed with p1772 wt, p1772e008, p1772e006, and pArray4 (targeting crArray4+Cas system) at MOIs from 100 to 0.0001. FIG. 10C is an inset of FIG. 10A and depicts a magnification of p1772e006 compared to pArray3 at an MOI of 0.0244 (top row) and 0.00610 (bottom row). FIG. 10D is a quantification of the fluorescence signal from the bacteria following infection with phage at a MOI of about 1.5 in FIG. 10A. FIG. 10E is a quantification of fluorescence signal from the bacteria following infection with phage at a MOI of about 1.5 in FIG. 10B.

FIG. 11 depicts the growth on an agar plate of P. aeruginosa cultures mixed with p1772 variants with different promoters. FIG. 11A shows the growth on an agar plate of P. aeruginosa cultures mixed with p1772 wt (wild type) and variants containing the same crArray 1 and Cas system, where the Cas system was driven by a different promoter, at an MOI of 100 to 0.00001. FIG. 11B shows a quantification of the fluorescence of the cells at MOI 1.5 from FIG. 11A.

FIG. 12A depicts a quantification of the fluorescence the growth on an agar plate of P. aeruginosa cultures mixed with p2132 wt (wild type) and p2132e002 (targeting crArray1+Cas system) at an MOI of 1.5. FIG. 12B depicts a quantification of the fluorescence from the growth on an agar plate of P. aeruginosa cultures mixed with p2973 wt (wild type) and p2973e002 (targeting crArray1+Cas system) at an MOI of 1.5.

FIG. 13 depicts the growth on an agar plate of different strains of P. aeruginosa cultures mixed different phage variants. p4209 wt (wild type) and p4209e002 (targeting crArray1+Cas system) were mixed with b2550 (Type I-E Cas), b2631 (Type I-F Cas), b2816 (Type I-E/I-F Cas), and b2825 (no active Type I Cas) strains of P. aeruginosa and plated after 0 hours, 3 hours or 24 hours of incubation.

FIG. 14 depicts the efficacy of the crArray/Cas insert in multiple P. aeruginosa strains. p4209 wt (wild type), p4209e001 (Cas system only) and p4209e002 (targeting crArray1+Cas system) were plagued on b2550 (Type I-E Cas), b2631 (Type I-F Cas), b2816 (Type I-E/I-F Cas), and b2825 (no active Type I Cas) strains of P. aeruginosa.

FIG. 15A-FIG. 15D exemplify in vivo efficacy results comparing p1772 wt (wild type) to p1772e005 (targeting crArray1+Cas system). FIG. 15A is a schematic depicting the experimental set-up for FIGS. 15B-15D. FIG. 15B depicts the efficacy of the phage when injected into mouse thigh muscle. The left panel depicts the number of colony forming units (CFU) recovered at 6 hours post-infection. The right panel depicts the number of plaque forming units (PFU) recovered at 6 hours post-infection. FIG. 15C depicts the efficacy of the phage when injected into mouse thigh muscle and depicts the number of CFU (top) and PFU (bottom) recovered at 8 and 24 hours post-infection. FIG. 15D depicts the efficacy of the phage when administered intravenously and depicts the number of CFU (top) and PFU (bottom) recovered 9, 12, 15 and 24 hours post infection. FIG. 15E depicts the experimental set-up for FIG. 15F. FIG. 15F depicts the dose response for treatment with p1772 wt and p1772e005 and depicts the amount of CFU (top) and PFU (bottom) recovered 8 and 24 hours post-infection. Data shown as mean±SEM. *p<0.05, **p<0.01, ***p<0.001, ****p<0.0001. One-way ANOVA with multiple comparisons or Two-way ANOVA with Tukey's test.

FIG. 16 is a schematic representation of the genome of wild type phage p004k and its engineered variant p004ke007. The bar below the genome axis indicates the region of the genome that was removed and replaced. The schematic below the phage genome illustrates the DNA that was used to replace WT phage genes in the deleted region.

FIG. 17 illustrates the efficacy of the crArray/Cas system insert in E. coli. FIG. 17A depicts the growth on an agar plate of p004kwt (wild type) and p004ke007 (crArray 5+Cas system) mixed with three strains of E. coli (b3402, b3418, or b4098). FIGS. 17B-17D are a quantification of the optical density of the cell growth in b3402, b3418, and b4098, respectively.

FIG. 18 exemplifies CRISPR-Cas3 engineered reference phage PB1e002 (crArray1+Cas system) acts cooperatively with p1772e005 (crArray1+Cas system).

FIG. 19 depicts the number of transformants produced after transfecting the Pseudomonas with inserts containing different spacer sequences.

### DETAILED DESCRIPTION

Disclosed herein, in certain embodiments, are bacteriophages comprising a nucleic acid sequence encoding a Type I CRISPR-Cas system comprising: (a) a CRISPR array (also referred to as “crArray”); (b) a Cascade polypeptide; and (c) a Cas3 polypeptide. Also disclosed herein, in certain embodiments, are pharmaceutical compositions comprising the bacteriophages disclosed herein. Further disclosed herein, in certain embodiments, are methods of killing a target bacterium comprising introducing into the target bacterium a bacteriophage comprising a nucleic acid sequence encoding a Type I CRISPR-Cas system comprising: (a) a CRISPR array; (b) a Cascade polypeptide; and (c) a Cas3 polypeptide. Further disclosed herein, in certain embodiments, are methods of treating a disease in an individual in need thereof, the method comprising administering to the individual a bacteriophage a nucleic acid sequence encoding a Type I CRISPR-Cas system comprising: (a) a CRISPR array; (b) a Cascade polypeptide; and (c) a Cas3 polypeptide

### Certain Terminology

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

Unless the context indicates otherwise, it is specifically intended that the various features of the disclosure described herein are able of being used in any combination. Moreover, the present disclosure also contemplates that in some embodiments, any feature or combination of features set forth herein are excluded or omitted. To illustrate, if the specification states that a composition comprises components A, B and C, it is specifically intended that any of A, B or C, or a combination thereof, are omitted and disclaimed singularly or in any combination.

One of skill in the art will understand the interchangeability of terms designating the various CRISPR-Cas systems and their components due to a lack of consistency in the literature and an ongoing effort in the art to unify such terminology.

As used in the description and the appended claims, the singular forms “a,” “an” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

The term “about” as used herein when referring to a measurable value such as a dosage or time period and the like refers to variations of ±20%, ±10%, ±5%, ±1%, +0.5%, or even ±0.1% of the specified amount. As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise”, “comprises”, and “comprising”, “includes”, “including”, “have” and “having”, as used herein, specify the presence of the stated features, steps, operations, elements, and/or components, but do not preclude the presence or addition of one or more other features, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed disclosure. Thus, the term “consisting essentially of” when used in a claim of this disclosure is not intended to be interpreted to be equivalent to “comprising.”

The term “consists of” and “consisting of”, as used herein, excludes any features, steps, operations, elements, and/or components not otherwise directly stated. The use of “consisting of” limits only the features, steps, operations, elements, and/or components set forth in that clause and does exclude other features, steps, operations, elements, and/or components from the claim as a whole.

The terms “complementary” or “complementarity”, as used herein, refer to the natural binding of polynucleotides under permissive salt and temperature conditions by base-pairing. For example, the sequence “A-G-T” binds to the complementary sequence “T-C-A.” Complementarity between two single-stranded molecules is “partial,” in which only some of the nucleotides bind, or it is complete when total complementarity exists between the single stranded molecules. The degree of complementarity between nucleic acid strands has significant effects on the efficiency and strength of hybridization between nucleic acid strands.

“Complement” as used herein means 100% complementarity or identity with the comparator nucleotide sequence or it means less than 100% complementarity (e.g., about 80%, 81%, 82%, 83%, 84%, 85%, 86%, 87%, 88%, 89%, 90%, 91%, 92%, 93%, 94%, 95%, 96%, 97%, 98%, 99%, and the like, complementarity). Complement or complementable may also be used in terms of a “complement” to or “complementing” a mutation.

As used herein, the term “CRISPR phage”, “CRISPR enhanced phage”, and “crPhage” refers to a bacteriophage particle comprising bacteriophage DNA comprising at least one heterologous polynucleotide that encodes at least one component of a CRISPR-Cas system (e.g., CRISPR array, crRNA; e.g., P1 bacteriophage comprising an insertion of a targeting crRNA). In some embodiments, the polynucleotide encodes at least one transcriptional activator of a CRISPR-Cas system. In some embodiments, the polynucleotide encodes at least one component of an anti-CRISPR polypeptide of a CRISPR-Cas system.

As used herein, the phrase “substantially identical,” or “substantial identity” in the context of two nucleic acid molecules, nucleotide sequences or protein sequences, refers to two or more sequences or subsequences that have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In some embodiments, substantial identity refers to two or more sequences or subsequences that have at least about 70%, at least about 75%, at least about 80%, at least about 85%, at least about 90%, at least about 95, 96, 96, 97, 98, or 99% identity. For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences is to a full-length polynucleotide sequence or to a portion thereof, or to a longer polynucleotide sequence. In some instances, “Percent identity” is determined using BLASTX version 2.0 for translated nucleotide sequences and BLASTN version 2.0 for polynucleotide sequences.

As used herein, a “target nucleotide sequence” refers to the portion of a target gene (i.e., target region in the genome or the “protospacer sequence,” which is adjacent to a protospacer adjacent motif (PAM) sequence) that is fully complementary or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a CRISPR array.

As used herein, the term “protospacer adjacent motif” or “PAM” refers to a DNA sequence present on the target DNA molecule adjacent to the nucleotide sequence matching the spacer sequence. This motif is found in the target gene next to the region to which a spacer sequence binds as a result of being complementary to that region and identifies the point at which base pairing with the spacer nucleotide sequence begins. The exact PAM sequence that is required varies between each different CRISPR-Cas system. Non-limiting examples of PAMs include CCA, CCT, CCG, TTC, AAG, AGG, ATG, GAG, and/or CC. In some instances, in Type I systems, the PAM is located immediately 5′ to the sequence that matches the spacer, and thus is 3′ to the sequence that base pairs with the spacer nucleotide sequence, and is directly recognized by Cascade. In some instances, for B. halodurans Type I-C systems, the PAM is YYC, where Y can be either T or C. In some instances, for the P. aeruginosa Type I-C system, the PAM is TTC. Once a cognate protospacer and PAM are recognized, Cas3 is recruited, which then cleaves and degrades the target DNA. For Type II systems, the PAM is required for a Cas9/sgRNA to form an R-loop to interrogate a specific DNA sequence through Watson-Crick pairing of its guide RNA with the genome. The PAM specificity is a function of the DNA-binding specificity of the Cas9 protein (e.g., a —protospacer adjacent motif recognition domain at the C-terminus of Cas9).

As used herein, the term “gene” refers to a nucleic acid molecule capable of being used to produce mRNA, tRNA, rRNA, miRNA, anti-microRNA, regulatory RNA, and the like. Genes may or may not be capable of being used to produce a functional protein or gene product. Genes include both coding and non-coding regions (e.g., introns, regulatory elements, promoters, enhancers, termination sequences and/or 5′ and 3′ untranslated regions). A gene is “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

By the terms “treat,” “treating,” or “treatment,” it is intended that the severity of the subject's condition is reduced or at least partially improved or modified and that some alleviation, mitigation or decrease in at least one clinical symptom is achieved, and/or there is a delay in the progression of the disease or condition, and/or delay of the onset of a disease or illness. With respect to an infection, a disease or a condition, the term refers to a decrease in the symptoms or other manifestations of the infection, disease or condition. In some embodiments, treatment provides a reduction in symptoms or other manifestations of the infection, disease or condition by at least about 5%, e.g., about 10%, 15%, 20%, 25%, 30%, 35%, 40%, 45%, 50% or more.

The terms “prevent,” “preventing,” and “prevention” (and grammatical variations thereof) refer to prevention and/or delay of the onset of an infection, disease, condition and/or a clinical symptom(s) in a subject and/or a reduction in the severity of the onset of the infection, disease, condition and/or clinical symptom(s) relative to what would occur in the absence of carrying out the methods disclosed herein prior to the onset of the disease, disorder and/or clinical symptom(s). Thus, in some embodiments, to prevent infection, food, surfaces, medical tools and devices are treated with compositions and by methods disclosed herein.

The terms with respect to an “infection”, “a disease”, or “a condition”, used herein, refer to any adverse, negative, or harmful physiological condition in a subject. In some embodiments, the source of an “infection”, “a disease”, or “a condition”, is the presence of a target bacterial population in and/or on a subject. In some embodiments, the bacterial population comprises one or more target bacterial species. In some embodiments, the one or more bacteria species in the bacterial population comprise one or more strains of one or more bacteria. In some embodiments, the target bacterial population causes an “infection”, “a disease”, or “a condition” that is acute or chronic. In some embodiments, the target bacterial population causes an “infection”, “a disease”, or “a condition” that is localized or systemic. In some embodiments, the target bacterial population causes an “infection”, “a disease”, or “a condition” that is idiopathic. In some embodiments, the target bacterial population causes an “infection”, “a disease”, or “a condition” that is acquired through means, including but not limited to, respiratory inhalation, ingestion, skin and wound infections, blood stream infections, middle-ear infections, gastrointestinal tract infections, peritoneal membrane infections, urinary tract infections, urogenital tract infections, oral soft tissue infections, intra-abdominal infections, epidermal or mucosal absorption, eye infections (including contact lens contamination), endocarditis, infections in cystic fibrosis, infections of indwelling medical devices such as joint prostheses, dental implants, catheters and cardiac implants, sexual contact, and/or hospital-acquired and ventilator-associated bacterial pneumonias.

The terms “individual”, or “subject” as used herein includes any animal that has or is susceptible to an infection, disease or condition involving bacteria. Thus, in some embodiments, subjects are mammals, avians, reptiles, amphibians, fish, crustaceans, or mollusks. Mammalian subjects include but are not limited to humans, non-human primates (e.g., gorilla, monkey, baboon, and chimpanzee, etc.), dogs, cats, goats, horses, pigs, cattle, sheep, and the like, and laboratory animals (e.g., rats, guinea pigs, mice, gerbils, hamsters, and the like). Avian subjects include but are not limited to chickens, ducks, turkeys, geese, quail, pheasants, and birds kept as pets (e.g., parakeets, parrots, macaws, cockatoos, canaries, and the like). Fish subjects include but are not limited to species used in aquaculture (e.g., tuna, salmon, tilapia, catfish, carp, trout, cod, bass, perch, snapper, and the like). Crustacean subjects include but are not limited to species used in aquaculture (e.g., shrimp, prawn, lobster, crayfish, crab and the like). Mollusk subjects include but are not limited to species used in aquaculture (e.g., abalone, mussel, oyster, clams, scallop and the like). In some embodiments, suitable subjects include both males and females and subjects of any age, including embryonic (e.g., in-utero or in-ovo), infant, juvenile, adolescent, adult and geriatric subjects. In some embodiments, a subject is a human.

As used here the term “isolated” in context of a nucleic acid sequence is a nucleic acid sequence that exists apart from its native environment.

As used herein, “expression cassette” means a recombinant nucleic acid molecule comprising a nucleotide sequence of interest (e.g., the recombinant nucleic acid molecules and CRISPR arrays disclosed herein), wherein the nucleotide sequence is operably associated with at least a control sequence (e.g., a promoter).

As used herein, “chimeric” refers to a nucleic acid molecule or a polypeptide in which at least two components are derived from different sources (e.g., different organisms, different coding regions).

As used herein, “selectable marker” means a nucleotide sequence that when expressed imparts a distinct phenotype to the host cell expressing the marker and thus allows such transformed cells to be distinguished from those that do not have the marker.

As used herein, “vector” refers to a composition for transferring, delivering or introducing a nucleic acid (or nucleic acids) into a cell.

As used herein, “pharmaceutically acceptable” means a material that is not biologically or otherwise undesirable, i.e., the material are administered to a subject without causing any undesirable biological effects such as toxicity.

As used herein the term “biofilm” means an accumulation of microorganisms embedded in a matrix of polysaccharide. Biofilms form on solid biological or non-biological surfaces and are medically important, accounting for over 80 percent of microbial infections in the body.

As used herein, the term “in vivo” is used to describe an event that takes place in a subject's body.

As used herein, the term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

### CRISPR/CAS Systems

CRISPR-Cas systems are naturally adaptive immune systems found in bacteria and archaea. The CRISPR system is a nuclease system involved in defense against invading phages and plasmids that provides a form of acquired immunity. There is a diversity of CRISPR-Cas systems based on the set of cas genes and their phylogenetic relationship. There are at least six different types (I through VI) where Type I represents over 50% of all identified systems in both bacteria and archaea. In some embodiments, a Type I, Type II, Type II, Type IV, Type V, or Type VI CRISPR-Cas system is used herein.

Type I systems are divided into seven subtypes including: Type I-A, Type I-B, Type I-C, Type I-D, Type I-E, Type I-F, and Type I-U. Type I CRISPR-Cas systems include a multi-subunit complex called Cascade (for complex associated with antiviral defense), Cas3 (a protein with nuclease, helicase, and exonuclease activity that is responsible for degradation of the target DNA), and CRISPR array encoding crRNA (stabilizes Cascade complex and directs Cascade and Cas3 to DNA target). Cascade forms a complex with the crRNA, and the protein-RNA pair recognizes its genomic target by complementary base pairing between the 5′ end of the crRNA sequence and a predefined protospacer. This complex is directed to homologous loci of pathogen DNA via regions encoded within the crRNA and protospacer-adjacent motifs (PAMs) within the pathogen genome. Base pairing occurs between the crRNA and the target DNA sequence leading to a conformational change. In the Type I-E system, the PAM is recognized by the CasA protein within Cascade, which then unwinds the flanking DNA to evaluate the extent of base pairing between the target and the spacer portion of the crRNA. Sufficient recognition leads Cascade to recruit and activate Cas3. Cas3 then nicks the non-target strand and begins degrading the strand in a 3′-to-5′ direction.

In the Type I-C system, the proteins Cas5, Cas8c, and Cas7 form the Cascade effector complex. Cas5 processes the pre-crRNA (which can take the form of a multi-spacer array, or a single spacer between two repeats) to produce individual crRNA(s) made up of a hairpin structure formed from the remaining repeat sequence and a linear spacer. The effector complex then binds to the processed crRNA and scans DNA to identify PAM sites. In the Type I-C system, the PAM is recognized by the Cas8c protein, which then acts to unwind the DNA duplex. If the sequence 3′ of the PAM matches the crRNA spacer that is bound to effector complex, a conformational change in the complex occurs and Cas3 is recruited to the site. Cas3 then nicks the non-target strand and begins degrading the DNA.

In some embodiments, the CRISPR-Cas system is endogenous to the target bacterium. In some embodiments, the CRISPR-Cas system is exogenous to the target bacterium. In some embodiments, the CRISPR-Cas system is a Type I CRISPR-Cas system. In some embodiments, the CRISPR-Cas system is a Type I-A CRISPR-Cas system. In some embodiments, the CRISPR-Cas system is a Type I-B CRISPR-Cas system. In some embodiments, the CRISPR-Cas system is a Type I-C CRISPR-Cas system. In some embodiments, the CRISPR-Cas system is a Type I-C CRISPR-Cas system derived from Pseudomonas aeruginosa. In some embodiments, the CRISPR-Cas system is a Type I-D CRISPR-Cas system. In some embodiments, the CRISPR-Cas system is a Type I-E CRISPR-Cas system. In some embodiments, the CRISPR-Cas system is a Type I-F CRISPR-Cas system. In some embodiments, the CRISPR-Cas system is a Type I-U CRISPR-Cas system. In some embodiments, the CRISPR-Cas system is a Type II CRISPR-Cas system. In some embodiments, the CRISPR-Cas system is a Type III CRISPR-Cas system.

In some embodiments, processing of a CRISPR-array disclosed herein includes, but is not limited to, the following processes: 1) transcription of the nucleic acid encoding a pre-crRNA; 2) recognition of the pre-crRNA by Cascade and/or specific members of Cascade, such as Cas6, and (3) processing of the pre-crRNA by Cascade or members of Cascade, such as Cas6, into mature crRNAs. In some embodiments, the mode of action for a Type I CRISPR system includes, but is not limited to, the following processes: 4) mature crRNA complexation with Cascade; 5) target recognition by the complexed mature crRNA/Cascade complex; and 6) nuclease activity at the target leading to DNA degradation.

### CRISPR Phages

Disclosed herein, in certain embodiments, are bacteriophage compositions comprising CRISPR-Cas systems and methods of use thereof.

Bacteriophages or “phages” represent a group of bacterial viruses and are engineered or sourced from environmental sources. Individual bacteriophage host ranges are usually narrow, meaning, phages are highly specific to one strain or few strains of a bacterial species and this specificity makes them unique in their antibacterial action. Bacteriophages are bacterial viruses that rely on the host's cellular machinery to replicate. Bacteriophages are generally classified as virulent or temperate phages depending on their lifestyle. Virulent bacteriophages, also known as lytic bacteriophages, can only undergo lytic replication. Lytic bacteriophages infect a host cell, undergo numerous rounds of replication, and trigger cell lysis to release newly made bacteriophage particles. In some embodiments, the lytic bacteriophages disclosed herein retain their replicative ability. In some embodiments, the lytic bacteriophages disclosed herein retain their ability to trigger cell lysis. In some embodiments, the lytic bacteriophages disclosed herein retain both they replicative ability and the ability to trigger cell lysis. In some embodiments, the bacteriophages disclosed herein comprise a CRISPR array. In some embodiments, the CRISPR array does not affect the bacteriophages ability to replicate and/or trigger cell lysis. Temperate or lysogenic bacteriophages can undergo lysogeny in which the phage stops replicating and stably resides within the host cell, either integrating into the bacterial genome or being maintained as an extrachromosomal plasmid. Temperate phages can also undergo lytic replication similar to their lytic bacteriophage counterparts. Whether a temperate phage replicates lytically or undergoes lysogeny upon infection depends on a variety of factors including growth conditions and the physiological state of the cell. A bacterial cell that has a lysogenic phage integrated into its genome is referred to as a lysogenic bacterium or lysogen. Exposure to adverse conditions may trigger reactivation of the lysogenic phage, termination of the lysogenic state and resumption of lytic replication by the phage. This process is called induction. Adverse conditions which favor the termination of the lysogenic state include desiccation, exposure to UV or ionizing radiation, and exposure to mutagenic chemicals. This leads to the expression of the phage genes, reversal of the integration process, and lytic multiplication. In some embodiments, the temperate bacteriophages disclosed herein are rendered lytic. The term “lysogeny gene” refers to any gene whose gene product promotes lysogeny of a temperate phage. Lysogeny genes can directly promote, as in the case of integrase proteins that facilitate integration of the bacteriophage into the host genome. Lysogeny genes can also indirectly promote lysogeny as in the case of CI transcriptional regulators which prevent transcription of genes required for lytic replication and thus favor maintenance of lysogeny.

Bacteriophages package and deliver synthetic DNA using three general approaches. Under the first approach, the synthetic DNA is recombined into the bacteriophage genome in a targeted manner, which usually involves a selectable marker. Under the second approach, restriction sites within the phage are used to introduce synthetic DNA in-vitro. Under the third approach, a plasmid generally encoding the phage packaging sites and lytic origin of replication is packaged as part of the assembly of the bacteriophage particle. The resulting plasmids have been coined “phagemids.”

Phages are limited to a given bacterial strain for evolutionary reasons. In some cases, injecting their genetic material into an incompatible strain is counterproductive. Phages have therefore evolved to specifically infect a limited cross-section of bacterial strains. However, some phages have been discovered that inject their genetic material into a wide range of bacteria. The classic example is the P1 phage, which has been shown to inject DNA in a range of gram-negative bacteria.

Disclosed herein, in some embodiments, are bacteriophages comprising a first nucleic acid sequence encoding a first spacer sequence or a crRNA transcribed therefrom, wherein the first spacer sequence is complementary to a target nucleotide sequence from a target gene in a target bacterium. In some embodiments, the bacteriophage comprises a first nucleic acid sequence encoding a first spacer sequence or a crRNA transcribed therefrom, wherein the first spacer sequence is complementary to a target nucleotide sequence from a target gene in a target bacterium, provided that the bacteriophage is rendered lytic. In some embodiments, the bacteriophage is a temperate bacteriophage. In some embodiments, the bacteriophage is rendered lytic by removal, replacement, or inactivation of a lysogenic gene. In some embodiments, the lysogenic gene plays a role in the maintenance of lysogenic cycle in the bacteriophage. In some embodiments, the lysogenic gene plays a role in establishing the lysogenic cycle in the bacteriophage. In some embodiments, the lysogenic gene plays a role in both establishing the lysogenic cycle and in the maintenance of the lysogenic cycle in the bacteriophage. In some embodiments, the lysogenic gene is a repressor gene. In some embodiments, the lysogenic gene is cI repressor gene. In some embodiments, the bacteriophage is rendered lytic by the removal of a regulatory element of a lysogeny gene. In some embodiments, the bacteriophage is rendered lytic by the removal of a promoter of a lysogeny gene. In some embodiments, the bacteriophage is rendered lytic by the removal of a functional element of a lysogeny gene. In some embodiments, the lysogenic gene is an activator gene. In some embodiments, the lysogenic gene is cII gene. In some embodiments, the lysogenic gene is lexA gene. In some embodiments, the lysogenic gene is int (integrase) gene. In some embodiments, two or more lysogeny genes are removed, replaced, or inactivated to cause arrest of a bacteriophage lysogeny cycle and/or induction of a lytic cycle. In some embodiments, the bacteriophage is rendered lytic via a second CRISPR array comprising a second spacer directed to a lysogenic gene. In some embodiments, the bacteriophage is rendered lytic by the insertion of one or more lytic genes. In some embodiments, the bacteriophage is rendered lytic by the insertion of one or more genes that contribute to the induction of a lytic cycle. In some embodiments, the bacteriophage is rendered lytic by altering the expression of one or more genes that contribute to the induction of a lytic cycle. In some embodiments, the bacteriophage phenotypically changes from a lysogenic bacteriophage to a lytic bacteriophage. In some embodiments, the phenotypic change is via a self-targeting CRISPR-Cas system to render a bacteriophage lytic since it is incapable of lysogeny. In some embodiments, the self-targeting CRISPR-Cas comprises a self-targeting crRNA from the prophage genome and kills lysogens. In some embodiments, the bacteriophage is rendered lytic by environmental alterations. In some embodiments, environmental alterations include, but are not limited to, alterations in temperature, pH, or nutrients, exposure to antibiotics, hydrogen peroxide, foreign DNA, or DNA damaging agents, presence of organic carbon, and presence of heavy metal (e.g. in the form of chromium (VI)). In some embodiments, the bacteriophage that is rendered lytic is prevented from reverting to lysogenic state. In some embodiments, the bacteriophage that is rendered lytic is prevented from reverting back to lysogenic state by way of introducing an additional CRISPR array. In some embodiments, the bacteriophage does not confer any new properties onto the target bacterium beyond cellular death cause by lytic activity of the bacteriophage and/or the activity of the CRISPR array. Further disclosed herein, in some embodiments, are temperate bacteriophages comprising a first nucleic acid sequence encoding a first spacer sequence or a crRNA transcribed therefrom, wherein the first spacer sequence is complementary to a target nucleotide sequence from a target gene in a target bacterium, provided the bacteriophage is rendered lytic. In some embodiments, the bacteriophage infects multiple bacterial strains. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence comprises at least a portion of an essential gene that is needed for survival of the target bacterium. In some embodiments, the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK. In some embodiments, the target nucleotide sequence is in a non-essential gene. In some embodiments, the target nucleotide sequence is a noncoding sequence. In some embodiments, the noncoding sequence is an intergenic sequence. In some embodiments, the spacer sequence is complementary to a target nucleotide sequence of a highly conserved sequence in a target bacterium. In some embodiments, the spacer sequence is complementary to a target nucleotide sequence of a sequence present in the target bacterium. In some embodiments, the spacer sequence is complementary to a target nucleotide sequence that comprises all or a part of a promoter sequence of the essential gene. In some embodiments, the first nucleic acid sequence comprises a first CRISPR array comprising at least one repeat sequence. In some embodiments, the at least one repeat sequence is operably linked to the first spacer sequence at either its 5′ end or its 3′ end. In some embodiments, the target bacterium is C. difficile.

In some embodiments, the bacteriophage or phagemid DNA is from a lysogenic or temperate bacteriophage. In some embodiments, the bacteriophages or phagemids include but are not limited to P1 phage, a M13 phage, a λ phage, a T4 phage, a T7 phage, a T7m phage, a ϕC2 phage, a ϕCD27 phage, a ϕNM1 phage, Bc431 v3 phage, ϕ10 phage, ϕ25 phage, Δ151 phage, A511-like phages, B054, 0176-like phages, or Campylobacter phages (such as NCTC 12676 and NCTC 12677). In some embodiments, the bacteriophage is ϕCD146 C. difficile bacteriophage. In some embodiments, the bacteriophage is ϕCD24-2 C. difficile bacteriophage.

In some embodiments, a plurality of bacteriophages are used together. In some embodiments, the plurality of bacteriophages used together targets the same or different bacteria within a sample or subject. In some embodiments, the bacteriophages used together comprises T4 phage, T7 phage, T7m phage, or any combination of bacteriophages described herein.

In some embodiments, bacteriophages of interest are obtained from environmental sources. or commercial research vendors. In some embodiments, obtained bacteriophages are screened for lytic activity against a library of bacteria and their associated strains. In some embodiments, the bacteriophages are screened against a library of bacteria and their associated strains for their ability to generate primary resistance in the screened bacteria.

In some embodiments, the nucleic acid is inserted into the bacteriophage genome. In some embodiments, the nucleic acid comprises a crArray, a Cas system, or a combination thereof. In some embodiments, the nucleic acid is inserted into the bacteriophage genome at a transcription terminator site at the end of an operon of interest. In some embodiments, the nucleic acid is inserted into the bacteriophage genome as a replacement for one or more removed non-essential genes. In some embodiments, the nucleic acid is inserted into the bacteriophage genome as a replacement for one or more removed lysogenic genes. In some embodiments, the replacement of non-essential and/or lysogenic genes with the nucleic acid enhances the lytic activity of the bacteriophage. In some embodiments, the replacement of non-essential and/or lysogenic genes with the nucleic acid renders a lysogenic bacteriophage lytic.

In some embodiments, the nucleic acid is introduced into the bacteriophage genome at a first location while one or more non-essential and/or lysogenic genes are separately removed and/or inactivated from the bacteriophage genome at a separate location. In some embodiments, the removal of one or more non-essential and/or lysogenic genes renders a lysogenic bacteriophage into a lytic bacteriophage. Similarly, in some embodiments, one or more lytic genes are introduced into the bacteriophage so as to render a non-lytic, lysogenic bacteriophage into a lytic bacteriophage.

In some embodiments, the replacement, removal, inactivation, or any combination thereof, of one or more non-essential and/or lysogenic genes is achieved by chemical, biochemical, and/or any suitable method. In some embodiments, the insertion of one or more lytic genes is achieved by any suitable chemical, biochemical, and/or physical method by homologous recombination.

In some embodiments, the bacteriophage is ϕCD146 C. difficile bacteriophage. In some embodiments, the bacteriophage is ϕCD24-2 C. difficile bacteriophage.

In some embodiments, the non-essential gene to be removed and/or replaced from the bacteriophage is a gene that is non-essential for the survival of the bacteriophage. In some embodiments, the non-essential gene to be removed and/or replaced from the bacteriophage is a gene that is non-essential for the induction and/or maintenance of lytic cycle. In some embodiments, the non-essential gene to be removed and/or replaced from the bacteriophage is gp49 from ϕCD146 C. difficile bacteriophage. In some embodiments, the non-essential gene to be removed and/or replaced from the bacteriophage is gp75 from ϕCD24-2 C. difficile bacteriophage.

Disclosed herein, in certain embodiments, are bacteriophages comprising a complete exogenous CRISPR-Cas system. In some embodiments, the CRISPR-Cas system is Type I CRISPR-Cas system, Type II CRISPR-Cas system, Type III CRISPR-Cas system, Type IV CRISPR-Cas system, Type V CRISPR-Cas system, or Type VI CRISPR-Cas system. Disclosed herein, in certain embodiments, are bacteriophages comprising a nucleic acid sequence encoding a Type I CRISPR-Cas system comprising: (a) a CRISPR array; (b) a Cascade polypeptide; and (c) a Cas3 polypeptide.

### CRISPR Array

In some embodiments, the CRISPR array (crArray) comprises a spacer sequence and at least one repeat sequence. In some embodiments, the CRISPR array encodes a processed, mature crRNA. In some embodiments, the mature crRNA is introduced into a phage or a target bacterium. In some embodiments, an endogenous or exogenous Cas6 processes the CRISPR array into mature crRNA. In some embodiments, an exogenous Cas6 is introduced into the phage. In some embodiments, the phage comprises an exogenous Cas6. In some embodiments, an exogenous Cas6 is introduced into a target bacterium.

In some embodiments, the CRISPR array comprises a spacer sequence. In some embodiments, the CRISPR array further comprises at least one repeat sequence. In some embodiments, the at least one repeat sequence is operably linked to the spacer sequence at either its 5′ end or its 3′ end. In some embodiments, the CRISPR array is of any length and comprises any number of spacer nucleotide sequences alternating with repeat nucleotide sequences necessary to achieve the desired level of killing of a target bacterium by targeting one or more essential genes. In some embodiments, the CRISPR array comprises, consists essentially of, or consists of 1 to about 100 spacer nucleotide sequences, each linked on its 5′ end and its 3′ end to a repeat nucleotide sequence. In some embodiments, the CRISPR array comprises, consists essentially of, or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, spacer nucleotide sequences.

### Spacer Sequence

In some embodiments, the spacer sequence is complementary to a target nucleotide sequence in a target bacterium. In some embodiments, the target nucleotide sequence is a coding region. In some embodiments, the coding region is an essential gene. In some embodiments, the coding region is a nonessential gene. In some embodiments, the target nucleotide sequence is a noncoding sequence. In some embodiments, the noncoding sequence is an intergenic sequence. In some embodiments, the spacer sequence is complementary to a target nucleotide sequence of a highly conserved sequence in a target bacterium. In some embodiments, the spacer sequence is complementary to a target nucleotide sequence of a sequence present in the target bacterium. In some embodiments, the spacer sequence is complementary to a target nucleotide sequence that comprises all or a part of a promoter sequence of the essential gene. In some embodiments, the spacer sequence comprises one, two, three, four, or five mismatches as compared to the target nucleotide sequence. In some embodiments, the mismatches are contiguous. In some embodiments, the mismatches are noncontiguous. In some embodiments, the spacer sequence has 70% complementarity to a target nucleotide sequence. In some embodiments, the spacer sequence has 80% complementarity to a target nucleotide sequence. In some embodiments, the spacer sequence is 85%, 90%, 95%, 96%, 97%, 98%, 99% or 100% complementarity to a target nucleotide sequence. In some embodiments, the spacer sequence has 100% complementarity to the target nucleotide sequence. In some embodiments, the spacer sequence has complete complementarity or substantial complementarity over a region of a target nucleotide sequence that are at least about 8 nucleotides to about 150 nucleotides in length. In some embodiments, a spacer sequence has complete complementarity or substantial complementarity over a region of a target nucleotide sequence that is at least about 20 nucleotides to about 100 nucleotides in length. In some embodiments, the 5′ region of the spacer sequence is 100% complementary to a target nucleotide sequence while the 3′ region of the spacer is substantially complementary to the target nucleotide sequence and therefore the overall complementarity of the spacer sequence to the target nucleotide sequence is less than 100%. For example, in some embodiments, the first 7, 8, 9, 10, 11, 12, 13, 14, 15, 16 nucleotides in the 3′ region of a 20 nucleotide spacer sequence (seed region) is 100% complementary to the target nucleotide sequence, while the remaining nucleotides in the 5′ region of the spacer sequence are substantially complementary (e.g., at least about 70% complementary) to the target nucleotide sequence. In some embodiments, the first 7 to 12 nucleotides of the 3′ end of the spacer sequence is 100% complementary to the target nucleotide sequence, while the remaining nucleotides in the 5′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target nucleotide sequence. In some embodiments, the first 7 to 10 nucleotides in the 3′ end of the spacer sequence is 75%-99% complementary to the target nucleotide sequence, while the remaining nucleotides in the 5′ region of the spacer sequence are at least about 50% to about 99% complementary to the target nucleotide sequence. In some embodiments, the first 7 to 10 nucleotides in the 3′ end of the spacer sequence is 100% complementary to the target nucleotide sequence, while the remaining nucleotides in the 5′ region of the spacer sequence are substantially complementary (e.g., at least about 70% complementary) to the target nucleotide sequence. In some embodiments, the first 10 nucleotides (within the seed region) of the spacer sequence is 100% complementary to the target nucleotide sequence, while the remaining nucleotides in the 5′ region of the spacer sequence are substantially complementary (e.g., at least about 70% complementary) to the target nucleotide sequence. In some embodiment, the 5′ region of a spacer sequence (e.g., the first 8 nucleotides at the 5′ end, the first 10 nucleotides at the 5′ end, the first 15 nucleotides at the 5′ end, the first 20 nucleotides at the 5′ end) have about 75% complementarity or more (75% to about 100% complementarity) to the target nucleotide sequence, while the remainder of the spacer sequence have about 50% or more complementarity to the target nucleotide sequence. In some embodiments, the first 8 nucleotides at the 5′ end of the spacer sequence have 100% complementarity to the target nucleotide sequence or have one or two mutations and therefore is about 88% complementary or about 75% complementary to the target nucleotide sequence, respectively, while the remainder of the spacer nucleotide sequence is at least about 50% or more complementary to the target nucleotide sequence.

In some embodiments, the spacer sequence is about 15 nucleotides to about 150 nucleotides in length. In some embodiments, the spacer nucleotide sequence is about 15 nucleotides to about 100 nucleotides in length (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nucleotides or more). In some embodiments, the spacer nucleotide sequence is a length of about 8 to about 150 nucleotides, about 8 to about 100 nucleotides, about 8 to about 50 nucleotides, about 8 to about 40 nucleotides, about 8 to about 30 nucleotides, about 8 to about 25 nucleotides, about 8 to about 20 nucleotides, about 10 to about 150 nucleotides, about 10 to about 100 nucleotides, about 10 to about 80 nucleotides, about 10 to about 50 nucleotides, about 10 to about 40, about 10 to about 30, about 10 to about 25, about 10 to about 20, about 15 to about 150, about 15 to about 100, about 15 to about 50, about 15 to about 40, about 15 to about 30, about 20 to about 150 nucleotides, about 20 to about 100 nucleotides, about 20 to about 80 nucleotides, about 20 to about 50 nucleotides, about 20 to about 40, about 20 to about 30, about 20 to about 25, at least about 8, at least about 10, at least about 15, at least about 20, at least about 25, at least about 30, at least about 32, at least about 35, at least about 40, at least about 44, at least about 50, at least about 60, at least about 70, at least about 80, at least about 90, at least about 100, at least about 110, at least about 120, at least about 130, at least about 140, at least about 150 nucleotides in length, or more, and any value or range therein. In some embodiments, the P. aeruginosa Type I-C Cas system has a spacer length of about 30 to 39 nucleotides, about 31 to about 38 nucleotides, about 32 to about 37 nucleotides, about 33 to about 36 nucleotides, about 34 to about 35 nucleotides, or about 35 nucleotides In some embodiments, the P. aeruginosa Type I-C Cas system has a spacer length of about 34 nucleotides. In some embodiments, the P. aeruginosa Type I-C Cas system has a spacer length of at least about 10, at least about 15, at least about 20, at least about 21, at least about 22, at least about 23, at least about 24, at least about 25, at least about 26, at least about 27, at least about 29, at least about 29, at least about 30, at least about 31, at least about 32, at least about 33, at least about 34, at least about, at least about 35, at least about 36, at least about 37, at least about 38, at least about 39, at least about 20, at least about 41, at least about 42, at least about 43, at least about 44, at least about 45, or more than about 45 nucleotides.

In some embodiments, the spacer sequence comprises at least or about 70%, 80%, 85%, 90%, 91%, 92%, 93%, 94%, 95%, 96%, 97%, 98%, 99%, or 100% sequence identity to any one of SEQ ID NOs: 12-22. In some instances, the spacer sequence comprises at least or about 95% homology to any one of SEQ ID NOS: 12-22. In some instances, the spacer sequence comprises at least or about 97% homology to any one of SEQ ID NOS: 12-22. In some instances, the spacer sequence comprises at least or about 99% homology to any one of SEQ ID NOS: 12-22. In some instances, the spacer sequence comprises 100% homology to any one of SEQ ID NOS: 12-22. In some instances, the spacer sequence comprises at least a portion having at least or about 3, 4, 5, 6, 7, 8, 9, 10, 12, 14, 16, 17, 18, 19, 20, 21, 22, 23, 24, 25, 26, 27, 28, 29, 30, 31, 32, 33, 34, or more than 34 nucleotides of any one of SEQ ID NOS: 12-22.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

The term “homology” or “similarity” between two proteins is determined by comparing the amino acid sequence and its conserved amino acid substitutes of one protein sequence to the second protein sequence. Similarity may be determined by procedures which are well-known in the art, for example, a BLAST program (Basic Local Alignment Search Tool at the National Center for Biological Information).

In some embodiments, the identity of two or more spacer sequences of the CRISPR array is the same. In some embodiments, the identity of two or more spacer sequences of the CRISPR array is different. In some embodiments, the identity of two or more spacer sequences of the CRISPR array is different but are complementary to one or more target nucleotide sequences. In some embodiments, the identity of two or more spacer sequences of the CRISPR array is different and are complementary to one or more target nucleotide sequences that are overlapping sequences. In some embodiments, the identity of two or more spacer sequences of the CRISPR array is different and are complementary to one or more target nucleotide sequences that are not overlapping sequences. In some embodiments, the target nucleotide sequence is about 10 to about 40 consecutive nucleotides in length located immediately adjacent to a PAM sequence (PAM sequence located immediately 3′ of the target region) in the genome of the organism. In some embodiments, a target nucleotide sequence is located adjacent to or flanked by a PAM (protospacer adjacent motif). In some embodiments, the two or more sequences of the CRISPR array comprises at least or about 70%, 80%, 85%, 90%, 91%, 92%, 93%, 94%, 95%, 96%, 97%, 98%, 99%, or 100% sequence identity to any one of SEQ ID NOs: 12-22. In some instances, the two or more sequences of the CRISPR array comprises at least or about 95% homology to any one of SEQ ID NOS: 12-22. In some instances, the two or more sequences of the CRISPR array comprises at least or about 97% homology to any one of SEQ ID NOS: 12-22. In some instances, the two or more sequences of the CRISPR array comprises at least or about 99% homology to any one of SEQ ID NOS: 12-22. In some instances, the two or more sequences of the CRISPR array comprises 100% homology to any one of SEQ ID NOS: 12-22. In some instances, the two or more sequences of the CRISPR array comprises at least a portion having at least or about 3, 4, 5, 6, 7, 8, 9, 10, 12, 14, 16, 17, 18, 19, 20, 21, 22, 23, 24, 25, 26, 27, 28, 29, 30, 31, 32, 33, 34, or more than 34 nucleotides of any one of SEQ ID NOS: 12-22.

In some embodiments, the CRISPR array comprises a first spacer sequence comprising at least or about 70%, 80%, 85%, 90%, 91%, 92%, 93%, 94%, 95%, 96%, 97%, 98%, 99%, or 100% sequence identity to any one of SEQ ID NOs: 12-15; a second spacer sequence comprising at least or about 70%, 80%, 85%, 90%, 91%, 92%, 93%, 94%, 95%, 96%, 97%, 98%, 99%, or 100% sequence identity to any one of SEQ ID NOs: 16-18; a third spacer sequence comprising at least or about 70%, 80%, 85%, 90%, 91%, 92%, 93%, 94%, 95%, 96%, 97%, 98%, 99%, or 100% sequence identity to any one of SEQ ID NOs: 19-21, wherein said first spacer sequence, second spacer sequence, and third spacer sequence comprise from 0-8 nucleotide modifications. In some instances, the first spacer sequence comprises at least or about 97% homology to any one of SEQ ID NOS: 12-15; the second spacer sequence comprises at least or about 97% homology to any one of SEQ ID NOS: 16-18; and the third spacer sequence comprises at least or about 97% homology to any one of SEQ ID NOS: 20-23. In some instances, the first spacer sequence comprises at least or about 99% homology to any one of SEQ ID NOS: 12-15; the second spacer sequence comprises at least or about 99% homology to any one of SEQ ID NOS: 16-18; and the third spacer sequence comprises at least or about 99% homology to any one of SEQ ID NOS: 19-21. In some instances, the first spacer sequence comprises at least or about 100% homology to any one of SEQ ID NOS: 12-15; the second spacer sequence comprises at least or about 100% homology to any one of SEQ ID NOS: 16-19; and the third spacer sequence comprises at least or about 100% homology to any one of SEQ ID NOS: 19-21.

In some embodiments, the CRISPR array comprises a first spacer sequence comprising at least or about 70%, 80%, 85%, 90%, 91%, 92%, 93%, 94%, 95%, 96%, 97%, 98%, 99%, or 100% sequence identity to SEQ ID NO: 12; a second spacer sequence comprising at least or about 70%, 80%, 85%, 90%, 91%, 92%, 93%, 94%, 95%, 96%, 97%, 98%, 99%, or 100% sequence identity to SEQ ID NO: 16; a third spacer sequence comprising at least or about 70%, 80%, 85%, 90%, 91%, 92%, 93%, 94%, 95%, 96%, 97%, 98%, 99%, or 100% sequence identity to SEQ ID NO: 20, wherein said first spacer sequence, second spacer sequence, and third spacer sequence comprise from 0-8 nucleotide modifications. In some instances, the first spacer sequence comprises at least or about 97% homology to SEQ ID NO: 12; the second spacer sequence comprises at least or about 97% homology to SEQ ID NO: 16; and the third spacer sequence comprises at least or about 97% homology to SEQ ID NO: 19. In some instances, the first spacer sequence comprises at least or about 99% homology to SEQ ID NO: 12; the second spacer sequence comprises at least or about 99% homology to SEQ ID NO: 16; and the third spacer sequence comprises at least or about 99% homology to SEQ ID NO: 19. In some instances, the first spacer sequence comprises at least or about 100% homology to SEQ ID NO: 12; the second spacer sequence comprises at least or about 100% homology to SEQ ID NO: 16; and the third spacer sequence comprises at least or about 100% homology to SEQ ID NO: 19.

In some embodiments, the CRISPR array comprises a first spacer sequence comprising at least or about 70%, 80%, 85%, 90%, 91%, 92%, 93%, 94%, 95%, 96%, 97%, 98%, 99%, or 100% sequence identity to SEQ ID NO: 13; a second spacer sequence comprising at least or about 70%, 80%, 85%, 90%, 91%, 92%, 93%, 94%, 95%, 96%, 97%, 98%, 99%, or 100% sequence identity to SEQ ID NO: 17; a third spacer sequence comprising at least or about 70%, 80%, 85%, 90%, 91%, 92%, 93%, 94%, 95%, 96%, 97%, 98%, 99%, or 100% sequence identity to SEQ ID NO: 20, wherein said first spacer sequence, second spacer sequence, and third spacer sequence comprise from 0-8 nucleotide modifications. In some instances, the first spacer sequence comprises at least or about 97% homology to SEQ ID NO: 13; the second spacer sequence comprises at least or about 97% homology to SEQ ID NO: 17; and the third spacer sequence comprises at least or about 97% homology to SEQ ID NO: 20. In some instances, the first spacer sequence comprises at least or about 99% homology to SEQ ID NO: 13; the second spacer sequence comprises at least or about 99% homology to SEQ ID NO: 17; and the third spacer sequence comprises at least or about 99% homology to SEQ ID NO: 20. In some instances, the first spacer sequence comprises at least or about 100% homology to SEQ ID NO: 13; the second spacer sequence comprises at least or about 100% homology to SEQ ID NO: 17; and the third spacer sequence comprises at least or about 100% homology to SEQ ID NO: 20.

In some embodiments, the CRISPR array comprises a first spacer sequence comprising at least or about 70%, 80%, 85%, 90%, 91%, 92%, 93%, 94%, 95%, 96%, 97%, 98%, 99%, or 100% sequence identity to SEQ ID NO: 14; a second spacer sequence comprising at least or about 70%, 80%, 85%, 90%, 91%, 92%, 93%, 94%, 95%, 96%, 97%, 98%, 99%, or 100% sequence identity to SEQ ID NO: 18; a third spacer sequence comprising at least or about 70%, 80%, 85%, 90%, 91%, 92%, 93%, 94%, 95%, 96%, 97%, 98%, 99%, or 100% sequence identity to SEQ ID NO: 21, wherein said first spacer sequence, second spacer sequence, and third spacer sequence comprise from 0-8 nucleotide modifications. In some instances, the first spacer sequence comprises at least or about 97% homology to SEQ ID NO: 14; the second spacer sequence comprises at least or about 97% homology to SEQ ID NO: 18; and the third spacer sequence comprises at least or about 97% homology to SEQ ID NO: 21. In some instances, the first spacer sequence comprises at least or about 99% homology to SEQ ID NO: 14; the second spacer sequence comprises at least or about 99% homology to SEQ ID NO: 18; and the third spacer sequence comprises at least or about 99% homology to SEQ ID NO: 21. In some instances, the first spacer sequence comprises at least or about 100% homology to SEQ ID NO: 14; the second spacer sequence comprises at least or about 100% homology to SEQ ID NO: 18; and the third spacer sequence comprises at least or about 100% homology to SEQ ID NO: 21.

The PAM sequence is found in the target gene next to the region to which a spacer sequence binds as a result of being complementary to that region and identifies the point at which base pairing with the spacer nucleotide sequence begins. The exact PAM sequence that is required varies between each different CRISPR-Cas system and is identified through established bioinformatics and experimental procedures. Non-limiting examples of PAMs include CCA, CCT, CCG, TTC, AAG, AGG, ATG, GAG, and/or CC. For Type I systems, the PAM is located immediately 5′ to the sequence that matches the spacer, and thus is 3′ to the sequence that base pairs with the spacer nucleotide sequence, and is directly recognized by Cascade. Once a protospacer is recognized, Cascade generally recruits the endonuclease Cas3, which cleaves and degrades the target DNA. For Type II systems, the PAM is required for a Cas9/sgRNA to form an R-loop to interrogate a specific DNA sequence through Watson-Crick pairing of its guide RNA with the genome. The PAM specificity is a function of the DNA-binding specificity of the Cas9 protein (e.g., a —protospacer adjacent motif recognition domain at the C-terminus of Cas9)

In some embodiments, the target nucleotide sequence in the bacterium to be killed is any essential target nucleotide sequence of interest. In some embodiments, the target nucleotide sequence is a non-essential sequence. In some embodiments, a target nucleotide sequence comprises, consists essentially of or consist of all or a part of a nucleotide sequence encoding a promoter, or a complement thereof, of the essential gene. In some embodiments, the spacer nucleotide sequence is complementary to a promoter, or a part thereof, of the essential gene. In some embodiments, the target nucleotide sequence comprises all or a part of a nucleotide sequence located on a coding or a non-coding strand of the essential gene. In some embodiments, the target nucleotide sequence comprises all or a part of a nucleotide sequence located on a coding of a transcribed region of the essential gene.

In some embodiments, the essential gene is any gene of an organism that is critical for its survival. However, being essential is highly dependent on the circumstances in which an organism lives. For instance, a gene required to digest starch is only essential if starch is the only source of energy. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence comprises at least a portion of an essential gene that is needed for survival of the target bacterium. In some embodiments, the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK. In some embodiments, a non-essential gene is any gene of an organism that is not critical for survival. However, being non-essential is highly dependent on the circumstances in which an organism lives.

In some embodiments, non-limiting examples of the target nucleotide sequence of interest includes a target nucleotide sequence encoding a transcriptional regulator, a translational regulator, a polymerase gene, a metabolic enzyme, a transporter, an RNase, a protease, a DNA replication enzyme, a DNA modifying or degrading enzyme, a regulatory RNA, a transfer RNA, or a ribosomal RNA. In some embodiments, the target nucleotide sequence is from a gene involved in cell-division, cell structure, metabolism, motility, pathogenicity, virulence, or antibiotic resistance. In some embodiments, the target nucleotide sequence is from a hypothetical gene whose function is not yet characterized. Thus, for example, these genes are any genes from any bacterium.

The appropriate spacer sequences for a full-construct phage may be identified by locating a search set of representative genomes, searching the genomes with relevant parameters, and determining the quality of a spacer for use in a CRISPR engineered phage.

First, a suitable search set of representative genomes is located and acquired for the organism/species/target of interest. The set of representative genomes may be found in a variety of databases, including without limitations the NCBI genbank or the PATRIC database. NCBI genbank is one of the largest databases available and contains a mixture of reference and submitted genomes for nearly every organism sequenced to date. Specifically, for pathogenic prokaryotes, the PATRIC (Pathosystems Resource Integration Center) database provides an additional comprehensive resource of genomes and provides a focus on clinically relevant strains and genomes relevant to a drug product. Both of the above databases allow for bulk downloading of genomes via FTP (File Transfer Protocol) servers, enabling rapid and programmatic dataset acquisition

Next, the genomes are searched with relevant parameters to locate suitable spacer sequences. Genomes may be read from start to end, in both the forward and reverse complement orientations, to locate contiguous stretches of DNA that contain a PAM (Protospacer Adjacent Motif) site. The spacer sequence will be the N-length DNA sequence 3′ or 5′ adjacent to the PAM site (depending on the CRISPR system type), where N is specific to the Cas system of interest and is generally known ahead of time. Characterizing the PAM sequence and spacer sequences may be performed during the discovery and initial research of a Cas system. Every observed PAM-adjacent spacer may be saved to a file and/or database for downstream use. The exact PAM sequence that is required varies between each different CRISPR-Cas system and is identified through established bioinformatics and experimental procedures.

Next, the quality of a spacer for use in a CRISPR engineered phage is determined. Each observed spacer may be evaluated to determine how many of the evaluated genomes they are present in. The observed spacers may be evaluated to see how many times they may occur in each given genome. Spacers that occur in more than one location per genome may be advantageous because the Cas system may not be able to recognize the target site if a mutation occurs, and each additional “backup” site increases the likelihood that a suitable, non-mutated target location will be present. The observed spacers may be evaluated to determine whether they occur in functionally annotated regions of the genome. If such information is available, the functional annotations may be further evaluated to determine whether those regions of the genome are “essential” for the survival and function of the organism. By focusing on spacers that occur in all, or nearly all, evaluated genomes of interest (>=99%), the spacer selection may be broadly applicable to many targeted genomes. Provided a large selection pool of conserved spacers exists, preference may be given to spacers that occur in regions of the genome that have known function, with higher preference given if those genomic regions are “essential” for survival and occur more than 1 time per genome.

The spacer sequences for a full construct phage, in some embodiments, are validated. In some embodiments, a first step comprises identifying a plasmid that replicates in the organism, species, or target of interest. In some embodiments, the plasmid has a selectable marker. In some embodiments, the selectable marker is an antibiotic-resistance gene. In some embodiments, an expression cassette includes a nucleotide sequence for a selectable marker. In some embodiments, the selectable marker is adenine deaminase (ada), blasticidin S deaminases (Bsr, BSD), bleomycin-binding protein (Ble), Neomycin phosphotransferase (neo), histidinol dehydrogenase (hisD), glutamine synthetase (GS), dihydrofolate reductase (dhfr), cytosine deaminase (codA), puromycin N-acetyltransferase (Pac), or hygromycin B phosphotransferase (Hph), ampicillin, chloramphenicol, kanamycin, tetracycline, polymyxin B, erythromycin, carbenicillin, streptomycin, spectinomycin, puromycin N-acetyltransferase (Pac), or zeocin (Sh bla). In some embodiments, the selectable marker is a gene involved in thymidylate synthase, thymidine kinase, dihydrofolate reductase, or glutamine synthetase. In some embodiments, the selectable marker is a gene encoding a fluorescent protein.

In some embodiments, a second step comprises inserting the genes encoding the Cas system into the plasmid such that they will be expressed in the organism, species, or target of interest. In some embodiments, a promoter is provided upstream of the Cas system. In some embodiments, the promoter is recognized by the organism, species, or target of interest to drive the expression of the Cas system. Exemplary promoters include, but are not limited to, L-arabinose inducible (araBAD, P BAD) promoter, any lac promoter, L-rhamnose inducible (rhaPBAD) promoter, T7 RNA polymerase promoter, trc promoter, tac promoter, lambda phage promoter (pLpL-9G-50), anhydrotetracycline-inducible (tetA) promoter, trp, Ipp, phoA, recA, proU, cst-1, cadA, nar, Ipp-lac, cspA, 11-lac operator, T3-lac operator, T4 gene 32, T5-lac operator, nprM-lac operator, Vhb, Protein A, corynebacterial-E. coli like promoters, thr, horn, diphtheria toxin promoter, sig A, sig B, nusG, SoxS, katb, a-amylase (Pamy), Ptms, P43 (comprised of two overlapping RNA polymerase a factor recognition sites, σA, σB), Ptms, P43, rplK-rplA, ferredoxin promoter, and/or xylose promoter. In some embodiments, the promoter is a BBa_J23102, BBa_J23104, or BBa_J23109. In some embodiments the promoter is derived from the organism, species, or target bacterium, such as endogenous CRISPR promoter, endogenous Cas operon promoter, p16, plpp, or ptat. In some embodiments, the promoter is a phage promoter, such as the promoter for gp105 or gp245. In some embodiments, a ribosomal binding site (RBS) is provided between the promoter and the Cas system. In some embodiments, the RBS is recognized by the organism, species, or target of interest.

In some embodiments, a third step comprises providing genome-targeting spacers into the plasmid. In some embodiments, the genome-targeting spacers are identified using bioinformatics. In some embodiments, the genome-targeting spacers are provided upstream of the repeat-spacer-repeat. In some embodiments, a promoter is provided. In some embodiments, the promoter is recognized by the organism, species, or target of interest to drive the expression of the crRNA. In some embodiments, the cloning for the third step comprises using an organism or species that is not targeted by the spacer being cloned.

In some embodiments, a fourth step comprises providing a non-target spacer into the plasmid that expresses the Cas system. In some embodiments, the non-target spacer comprises a sequence is random. In some embodiments, the non-target spacer comprises a sequence that does not comprise targeting sites in the genome of the organism, species, or target of interest. In some embodiments, the non-target spacer sequence is determined using bioinformatics to not comprise targeting sites in the genome of the organism, species, or target of interest. In some embodiments, the non-target spacer sequence is provided upstream of the repeat-spacer-repeat. In some embodiments, a promoter is provided. In some embodiments, the promoter is recognized by the organism, species, or target of interest to drive the expression of the crRNA.

In some embodiments, a fifth step comprises determining an efficacy of each spacer generated. In some embodiments, the killing efficacy is determined. In some embodiments, the efficacy of each spacer at targeting the bacterial genome is determined. In some embodiments, the plasmids comprising the spacer comprises about 0.5-fold, about 1-fold, 5-fold, 10-fold, 20-fold, 40-fold, 60-fold, 80-fold, or up to about 100 fold reduction in transfer rate as compared to a plasmid that comprises the non-targeting spacer.

### Repeat Nucleotide Sequences

In some embodiments, a repeat nucleotide sequence of the CRISPR array comprises a nucleotide sequence of any known repeat nucleotide sequence of a CRISPR-Cas system. In some embodiments, a repeat nucleotide sequence is of a synthetic sequence comprising the secondary structure of a native repeat from a CRISPR-Cas system (e.g., an internal hairpin). In some embodiments, the repeat nucleotide sequences are distinct from one another based on the known repeat nucleotide sequences of a CRISPR-Cas system. In some embodiments, the repeat nucleotide sequences are each composed of distinct secondary structures of a native repeat from a CRISPR-Cas system (e.g., an internal hairpin). In some embodiments, the repeat nucleotide sequences are a combination of distinct repeat nucleotide sequences operable with a CRISPR-Cas system.

In some embodiments, the spacer sequence is linked at its 5′ end to the 3′ end of a repeat sequence. In some embodiments, the spacer sequence is linked at its 5′ end to about 1 to about 8, about 1 to about 10, or about 1 to about 15 nucleotides of the 3′ end of a repeat sequence. In some embodiments, the about 1 to about 8, about 1 to about 10, about 1 to about 15 nucleotides of the repeat sequence are a portion of the 3′ end of a repeat sequence. In some embodiments, the spacer nucleotide sequence is linked at its 3′ end to the 5′ end of a repeat sequence. In some embodiments, the spacer is linked at its 3′ end to about 1 to about 8, about 1 to about 10, or about 1 to about 15 nucleotides of the 5′ end of a repeat sequence. In some embodiments, the about 1 to about 8, about 1 to about 10, about 1 to about 15 nucleotides of the repeat sequence are a portion of the 5′ end of a repeat sequence.

In some embodiments, the spacer nucleotide sequence is linked at its 5′ end to a first repeat sequence and linked at its 3′ end to a second repeat sequence to form a repeat-spacer-repeat sequence. In some embodiments, the spacer sequence is linked at its 5′ end to the 3′ end of a first repeat sequence and is linked at its 3′ end to the 5′ of a second repeat sequence where the spacer sequence and the second repeat sequence are repeated to form a repeat-(spacer-repeat)n sequence such that n is any integer from 1 to 100. In some embodiments, a repeat-(spacer-repeat)n sequence comprises, consists essentially of, or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, spacer nucleotide sequences.

In some embodiments, the repeat sequence is identical to or substantially identical to a repeat sequence from a wild-type CRISPR loci. In some embodiments, the repeat sequence is a repeat sequence found in Table 3. In some embodiments, the repeat sequence is a sequence described herein. In some embodiments, the repeat sequence comprises a portion of a wild type repeat sequence (e.g., 1, 2, 3, 4, 5, 6, 7, 8, 9, 10, 11, 12, 13, 14, 15 or more contiguous nucleotides of a wild type repeat sequence). In some embodiments, the repeat sequence comprises, consists essentially of, or consists of at least one nucleotide (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, or more nucleotides, or any range therein). In some embodiments, the repeat sequence comprises, consists essentially of, or consists of no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 nucleotides. In some embodiments, the repeat sequence comprises about 20 to 40, 21 to 40, 22 to 40 23 to 40, 24 to 40, 25 to 40, 26 to 40, 27 to 40, 28 to 40, 29 to 40, 30 to 30, 31 to 40, 32 to 40, 33 to 40, 34 to 40, 35 to 40, 36 to 40, 37 to 40, 38 to 40, 39 to 40, 20 to 39, 20 to 38, 20 to 37, 20 to 36, 20 to 35, 20 to 34, 20 to 33, 20 to 32, 20 to 31, 20 to 30, 20 to 29, 20 to 28, 20 to 26, 20 to 25, 20 to 24, 20 to 23, 20 to 22, or 20 to 21 nucleotides. In some embodiments, the repeat sequence comprises about 20 to 35, 21 to 35, 22 to 35 23 to 35, 24 to 35, 25 to 35, 26 to 35, 27 to 35, 28 to 35, 29 to 35, 30 to 30, 31 to 35, 32 to 35, 33 to 35, 34 to 35, 25 to 40, 25 to 39, 25 to 38, 25 to 37, 25 to 36, 25 to 35, 25 to 34, 25 to 33, 25 to 32, 25 to 31, 25 to 30, 25 to 29, 25 to 28, 25 to 26 nucleotides. In some embodiments, the system is a P. aeruginosa Type I-C Cas system. In some embodiments, the P. aeruginosa Type I-C Cas system has a repeat length of about 25 to 38 nucleotides.

In some embodiments, the repeat sequence comprises at least or about 70%, 80%, 85%, 90%, 91%, 92%, 93%, 94%, 95%, 96%, 97%, 98%, 99%, or 100% sequence identity to any one of SEQ ID NOs: 24-28. In some instances, the repeat sequence comprises at least or about 95% homology to any one of SEQ ID NOS: 24-28. In some instances, the repeat sequence comprises at least or about 97% homology to any one of SEQ ID NOS: 24-28. In some instances, the repeat sequence comprises at least or about 99% homology to any one of SEQ ID NOS: 24-28. In some instances, the repeat sequence comprises 100% homology to any one of SEQ ID NOS: 24-28. In some instances, the repeat sequence comprises at least a portion having at least or about 3, 4, 5, 6, 7, 8, 9, 10, 12, 14, 16, 17, 18, 19, 20, 21, 22, 23, 24, 25, 26, 27, 28, 29, 30, 31, 32, or more than 32 nucleotides of any one of SEQ ID NOS: 24-28.

### Transcriptional Activators

In some embodiments, the nucleic acid sequence further comprises a transcriptional activator. In some embodiments, the transcriptional activator encoded regulates the expression of genes of interest within the target bacterium. In some embodiments, the transcriptional activator activates the expression of genes of interest within the target bacterium whether exogenous or endogenous. In some embodiments, the transcriptional activator activates the expression genes of interest within the target bacterium by disrupting the activity of one or more inhibitory elements within the target bacterium. In some embodiments, the inhibitory element comprises a transcriptional repressor. In some embodiments, the inhibitory element comprises a global transcriptional repressor. In some embodiments the inhibitory element is a histone-like nucleoid-structuring (H-NS) protein or homologue or functional fragment thereof. In some embodiments, the inhibitory element is a leucine responsive regulatory protein (LRP). In some embodiments, the inhibitory element is a CodY protein.

In some bacteria, the CRISPR-Cas system is poorly expressed and considered silent under most environmental conditions. In these bacteria, the regulation of the CRISPR-Cas system is the result of the activity of transcriptional regulators, for example histone-like nucleoid-structuring (H-NS) protein which is widely involved in transcriptional regulation of the host genome. H-NS exerts control over host transcriptional regulation by multimerization along AT-rich sites resulting in DNA bending. In some bacteria, such as E. coli, the regulation of the CRISPR-Cas3 operon is regulated by H-NS.

Similarly, in some bacteria, the repression of the CRISPR-Cas system is controlled by an inhibitory element, for example the leucine responsive regulatory protein (LRP). LRP has been implicated in binding to upstream and downstream regions of the transcriptional start sites. Notably, the activity of LRP in regulating expression of the CRISPR-Cas system varies from bacteria to bacteria. Unlike, H-NS which has broad inter-species repression activity, LRP has been shown to differentially regulate the expression of the host CRISPR-Cas system. As such, in some instances, LRP reflects a host-specific means of regulating CRISPR-Cas system expression in different bacteria.

In some instances, the repression of CRISPR-Cas system is also controlled by inhibitory element CodY. CodY is a GTP-sensing transcriptional repressor that acts through DNA binding. The intracellular concentration of GTP acts as an indicator for the environmental nutritional status. Under normal culture conditions, GTP is abundant and binds with CodY to repress transcriptional activity. However, as GTP concentrations decreases, CodY becomes less active in binding DNA, thereby allowing transcription of the formerly repressed genes to occur. As such, CodY acts as a stringent global transcriptional repressor.

In some embodiments, the transcriptional activator is a LeuO polypeptide, any homolog or functional fragment thereof, a leu coding sequence, or an agent that upregulates LeuO. In some embodiments, the transcriptional activator comprises any ortholog or functional equivalent of LeuO. In some bacteria, LeuO acts in opposition to H-NS by acting as a global transcriptional regulator that responds to environmental nutritional status of a bacterium. Under normal conditions, LeuO is poorly expressed. However, under amino acid starvation and/or reaching of the stationary phase in the bacterial life cycle, LeuO is upregulated. Increased expression of LeuO leads to it antagonizing H-NS at overlapping promoter regions to effect gene expression. Overexpression of LeuO upregulates the expression of the CRISPR-Cas system. In E. coli and S. typhimurium, LeuO drives increased expression of the casABCDE operon which has predicted LeuO and H-NS binding sequences upstream of CasA.

In some embodiments, the expression of LeuO leads to disruption of an inhibitory element. In some embodiments, the disruption of an inhibitory element due to expression of LeuO removes the transcriptional repression of a CRISPR-Cas system. In some embodiments, the expression of LeuO removes transcriptional repression of a CRISPR-Cas system due to activity of H-NS. In some embodiments, the disruption of an inhibitory element due to the expression of LeuO causes an increase in the expression of a CRISPR-Cas system. In some embodiments, the increase in the expression of a CRISPR-Cas system due to the disruption of an inhibitory element caused by the expression of LeuO causes an increase in the CRISPR-Cas processing of a nucleic acid sequence comprising a CRISPR array. In some embodiments, the increase in the expression of a CRISPR-Cas system due to the disruption of an inhibitory element by the expression of LeuO causes an increase in the CRISPR-Cas processing of a nucleic acid sequence comprising a CRISPR array so as to increase the level of lethality of the CRISPR array against a bacterium. In some embodiments, transcriptional activator causes increase activity of a bacteriophage and/or the CRISPR-Cas system.

### Regulatory Elements

In some embodiments, the nucleic acid sequences are operatively associated with a variety of promoters, terminators and other regulatory elements for expression in various organisms or cells. In some embodiments, the nucleic acid sequence further comprises a leader sequence. In some embodiments, the nucleic acid sequence further comprises a promoter sequence. In some embodiments, at least one promoter and/or terminator is operably linked the CRISPR array. Any promoter useful with this disclosure is used and includes, for example, promoters functional with the organism of interest as well as constitutive, inducible, developmental regulated, tissue-specific/preferred-promoters, and the like, as disclosed herein. A regulatory element as used herein is endogenous or heterologous. In some embodiments, an endogenous regulatory element derived from the subject organism is inserted into a genetic context in which it does not naturally occur (e.g. a different position in the genome than as found in nature), thereby producing a recombinant or non-native nucleic acid.

In some embodiments, expression of the nucleic acid sequence is constitutive, inducible, temporally regulated, developmentally regulated, or chemically regulated. In some embodiments, the expression of the nucleic acid sequence is made constitutive, inducible, temporally regulated, developmentally regulated, or chemically regulated by operatively linking the nucleic acid sequence to a promoter functional in an organism of interest. In some embodiments, repression is made reversible by operatively linking the nucleic acid sequence to an inducible promoter that is functional in an organism of interest. The choice of promoter disclosed herein varies depending on the quantitative, temporal and spatial requirements for expression, and also depending on the host cell to be transformed.

Exemplary promoters for use with the methods, bacteriophages and compositions disclosed herein include promoters that are functional in bacteria. For example, L-arabinose inducible (araBAD, P BAD) promoter, any lac promoter, L-rhamnose inducible (rhaPBAD) promoter, T7 RNA polymerase promoter, trc promoter, tac promoter, lambda phage promoter (pLpL-9G-50), anhydrotetracycline-inducible (tetA) promoter, trp, Ipp, phoA, recA, proU, cst-1, cadA, nar, Ipp-lac, cspA, 11-lac operator, T3-lac operator, T4 gene 32, T5-lac operator, nprM-lac operator, Vhb, Protein A, corynebacterial-E. coli like promoters, thr, horn, diphtheria toxin promoter, sig A, sig B, nusG, SoxS, katb, a-amylase (Pamy), Ptms, P43 (comprised of two overlapping RNA polymerase a factor recognition sites, σA, σB), Ptms, P43, rplK-rplA, ferredoxin promoter, and/or xylose promoter. In some embodiments, the promoter is a BBa_J23102 promoter. In some embodiments, the promoter works in a broad range of bacteria, such as BBa_J23104, BBa_J23109. In some embodiments the promoter is derived from the target bacterium, such as endogenous CRISPR promoter, endogenous Cas operon promoter, p16, plpp, or ptat. In some embodiments, the promoter is a phage promoter, such as the promoter for gp105 or gp245.

In some embodiments, the promoter comprises at least or about 70%, 80%, 85%, 90%, 91%, 92%, 93%, 94%, 95%, 96%, 97%, 98%, 99%, or 100% sequence identity to any one of SEQ ID NOs: 1-11. In some instances, the promoter comprises at least or about 95% homology to any one of SEQ ID NOS: 1-11. In some instances, the promoter comprises at least or about 97% homology to any one of SEQ ID NOS: 1-11. In some instances, the promoter comprises at least or about 99% homology to any one of SEQ ID NOS: 1-11. In some instances, the promoter comprises 100% homology to any one of SEQ ID NOS: 1-11. In some instances, the promoter comprises at least a portion having at least or about 3, 4, 5, 6, 7, 8, 9, 10, 12, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more than 50 nucleotides of any one of SEQ ID NOS: 1-11. In some instances, the promoter comprises at least a portion having at least or about 50, 55, 60, 65, 70, 75, 80, 85, 90, 95, 100, 105, 110, 115 120, 125, 130, 135, 140, 145, 150, 155, 160, 165, 170, 175, 180, 185, 190, 195, 200, 205, 210, 215, or more than 215 nucleotides of any one of SEQ ID NOS: 1-11.

In some embodiments, inducible promoters are used. In some embodiments, chemical-regulated promoters are used to modulate the expression of a gene in an organism through the application of an exogenous chemical regulator. The use of chemically regulated promoters enables RNAs and/or the polypeptides encoded by the nucleic acid sequence to be synthesized only when, for example, an organism is treated with the inducing chemicals. In some embodiments where a chemical-inducible promoter is used, the application of a chemical induces gene expression. In some embodiments wherein a chemical-repressible promoter is used, the application of the chemical represses gene expression. In some embodiments, the promoter is a light-inducible promoter, where application of specific wavelengths of light induces gene expression. In some embodiments, a promoter is a light-repressible promoter, where application of specific wavelengths of light represses gene expression.

### Expression Cassette

In some embodiments, the nucleic acid sequence is an expression cassette or in an expression cassette. In some embodiments, the expression cassettes are designed to express the nucleic acid sequence disclosed herein. In some embodiments, the nucleic acid sequence is an expression cassette encoding components of a CRISPR-Cas system. In some embodiments, the nucleic acid sequence is an expression cassette encoding components of a Type I CRISPR-Cas system. In some embodiments, the nucleic acid sequence is an expression cassette encoding an operable CRISPR-Cas system. In some embodiments, the nucleic acid sequence is an expression cassette encoding the operable components of a Type I CRISPR-Cas system, including Cascade and Cas3. In some embodiments, the nucleic acid sequence is an expression cassette encoding the operable components of a Type I CRISPR-Cas system, including a crRNA, Cascade and Cas3.

In some embodiments, an expression cassette comprising a nucleic acid sequence of interest is chimeric, meaning that at least one of its components is heterologous with respect to at least one of its other components. In some embodiments, an expression cassette is naturally occurring but has been obtained in a recombinant form useful for heterologous expression.

In some embodiments, an expression cassette includes a transcriptional and/or translational termination region (i.e. termination region) that is functional in the selected host cell. In some embodiments, termination regions are responsible for the termination of transcription beyond the heterologous nucleic acid sequence of interest and for correct mRNA polyadenylation. In some embodiments, the termination region is native to the transcriptional initiation region, is native to the operably linked nucleic acid sequence of interest, is native to the host cell, or is derived from another source (i.e., foreign or heterologous to the promoter, to the nucleic acid sequence of interest, to the host, or any combination thereof). In some embodiments, terminators are operably linked to the nucleic acid sequence disclosed herein.

In some embodiments, an expression cassette includes a nucleotide sequence for a selectable marker. In some embodiments, the nucleotide sequence encodes either a selectable or a screenable marker, depending on whether the marker confers a trait that is selected for by chemical means, such as by using a selective agent (e.g. an antibiotic), or on whether the marker is simply a trait that one identifies through observation or testing, such as by screening (e.g., fluorescence).

### Vectors

In addition to expression cassettes, the nucleic acid sequences disclosed herein (e.g. nucleic acid sequence comprising a CRISPR array) are used in connection with vectors. A vector comprises a nucleic acid molecule comprising the nucleotide sequence(s) to be transferred, delivered or introduced. Non-limiting examples of general classes of vectors include, but are not limited to, a viral vector, a plasmid vector, a phage vector, a phagemid vector, a cosmid vector, a fosmid vector, a bacteriophage, an artificial chromosome, or an agrobacterium binary vector in double or single stranded linear or circular form which may or may not be self-transmissible or mobilizable. A vector transforms prokaryotic or eukaryotic host either by integration into the cellular genome or exist extrachromosomally (e.g. autonomous replicating plasmid with an origin of replication). Additionally, included are shuttle vectors by which is meant a DNA vehicle capable, naturally or by design, of replication in two different host organisms. In some embodiments, a shuttle vector replicates in actinomycetes and bacteria and/or eukaryotes. In some embodiments, the nucleic acid in the vector are under the control of, and operably linked to, an appropriate promoter or other regulatory elements for transcription in a host cell. In some embodiments, the vector is a bi-functional expression vector which functions in multiple hosts.

### Codon Optimization

In some embodiments, the nucleic acid sequence is codon optimized for expression in any species of interest. Codon optimization involves modification of a nucleotide sequence for codon usage bias using species-specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications of the nucleotide sequences are determined by comparing the species-specific codon usage table with the codons present in the native polynucleotide sequences. Codon optimization of a nucleotide sequence results in a nucleotide sequence having less than 100% identity (e.g., 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to the native nucleotide sequence but which still encodes a polypeptide having the same function as that encoded by the original nucleotide sequence. In some embodiments, the nucleic acid sequences of this disclosure are codon optimized for expression in the organism/species of interest.

### Transformation

In some embodiments, the nucleic acid sequence, and/or expression cassettes disclosed herein are expressed transiently and/or stably incorporated into the genome of a host organism. In some embodiments, a the nucleic acid sequence and/or expression cassettes disclosed herein is introduced into a cell by any method known to those of skill in the art. Exemplary methods of transformation include transformation via electroporation of competent cells, passive uptake by competent cells, chemical transformation of competent cells, as well as any other electrical, chemical, physical (mechanical) and/or biological mechanism that results in the introduction of nucleic acid into a cell, including any combination thereof. In some embodiments, transformation of a cell comprises nuclear transformation. In some embodiments, transformation of a cell comprises plasmid transformation and conjugation.

In some embodiments, when more than one nucleic acid sequence is introduced, the nucleotide sequences are assembled as part of a single nucleic acid construct, or as separate nucleic acid constructs, and are located on the same or different nucleic acid constructs. In some embodiments, nucleotide sequences are introduced into the cell of interest in a single transformation event, or in separate transformation events.

### Type I CRISPR-Cas System

In some embodiments, the Type I CRISPR-Cas system is a Type I-A system, Type I-B system, Type I-C system, Type I-D system, Type I-E system, or Type I-F system. In some embodiments, the Type I CRISPR-Cas system is a Type I-A system. In some embodiments, the Type I CRISPR-Cas system is a Type I-B system. In some embodiments, the Type I CRISPR-Cas system is a Type I-C system. In some embodiments, the Type I CRISPR-Cas system is a Type I-D system. In some embodiments, the Type I CRISPR-Cas system is a Type I-E system. In some embodiments, the Type I CRISPR-Cas system is a Type I-F system. In some embodiments, the Type I CRISPR-Cas system comprises Cascade polypeptides. Type I Cascade polypeptides process CRISPR arrays to produce a processed RNA that is then used to bind the complex to a target sequence that is complementary to the spacer in the processed RNA. In some embodiments, the Type I Cascade complex is a Type I-A Cascade polypeptides, a Type I-B Cascade polypeptides, a Type I-C Cascade polypeptides, a Type I-D Cascade polypeptides, a Type I-E Cascade polypeptides, a Type I-F Cascade polypeptides, or a Type I-U Cascade polypeptides.

In some embodiments, the Type I Cascade complex comprises: (a) a nucleotide sequence encoding a Cas7 (Csa2) polypeptide, a nucleotide sequence encoding a Cas8a1 (Csx13) polypeptide or a Cas8a2 (Csx9) polypeptide, a nucleotide sequence encoding a Cas5 polypeptide, a nucleotide sequence encoding a Csa5 polypeptide, a nucleotide sequence encoding a Cas6a polypeptide, a nucleotide sequence encoding a Cas3′ polypeptide, and a nucleotide sequence encoding a Cas3″ polypeptide having no nuclease activity (Type I-A); (b) a nucleotide sequence encoding a Cas6b polypeptide, a nucleotide sequence encoding a Cas8b (Csh1) polypeptide, a nucleotide sequence encoding a Cas7 (Csh2) polypeptide, and a nucleotide sequence encoding a Cas5 polypeptide (Type I-B); (c) a nucleotide sequence encoding a Cas5d polypeptide, a nucleotide sequence encoding a Cas8c (Csd1) polypeptide, and a nucleotide sequence encoding a Cas7 (Csd2) polypeptide (Type I-C); (d) a nucleotide sequence encoding a Cas10d (Csc3) polypeptide, a nucleotide sequence encoding a Csc2 polypeptide, a nucleotide sequence encoding a Csc1 polypeptide, and a nucleotide sequence encoding a Cas6d polypeptide (Type I-D); (e) a nucleotide sequence encoding a Cse1 (CasA) polypeptide, a nucleotide sequence encoding a Cse2 (CasB) polypeptide, a nucleotide sequence encoding a Cas7 (CasC) polypeptide, a nucleotide sequence encoding a Cas5 (CasD) polypeptide, and a nucleotide sequence encoding a Cas6e (CasE) polypeptide (Type I-E); and/or (f) a nucleotide sequence encoding a Cys1 polypeptide, a nucleotide sequence encoding a Cys2 polypeptide, a nucleotide sequence encoding a Cas7 (Cys3) polypeptide, and a nucleotide sequence encoding a Cas6f polypeptide (Type I-F).

In some embodiments, the Type I CRISPR-Cas system is exogenous to the target bacterium.

### Target Bacterium

In some embodiments, the target bacterium comprises one or more species of the target bacterium. In some embodiments, the target bacterium comprises one or more strains of the target bacterium. In some embodiments, non-limiting examples of target bacteria include Escherichia spp., Salmonella spp., Bacillus spp., Corynebacterium spp., Clostridium spp., Clostridioides spp., Pseudomonas spp., Lactococcus spp., Acinetobacter spp., Mycobacterium spp., Myxococcus spp., Staphylococcus spp., Streptococcus spp., Enterococcus spp., Bacteroides spp., Fusobacterium spp., Actinomyces spp., Porphyromonas spp., or cyanobacteria. In some embodiments, non-limiting examples of bacteria include Escherichia coli, Salmonella enterica, Bacillus subtilis, Clostridium acetobutylicum, Clostridium ljungdahlii, Clostridioides difficile, Clostridium bolteae, Acinetobacter baumannii, Mycobacterium tuberculosis, Mycobacterium abscessus, Mycobacterium intracellulare, Mycobacterium fortuitum, Mycobacterium chelonae, Mycobacterium avium, Mycobacterium gordonae, Myxococcus xanthus, Streptococcus pyogenes, or cyanobacteria. In some embodiments, non-limiting examples of bacteria include Staphylococcus aureus, methicillin resistant Staphylococcus aureus, Streptococcus pneumoniae, carbapenem-resistant Enterobacteriaceae, extended spectrum beta-lactamase (ESBL)-producing Enterobacteriaceae, Staphylococcus epidermidis, Staphylococcus salivarius, Corynebacterium minutissimum, Corynebacterium pseudodiphtheriticum, Corynebacterium striatum, Corynebacterium group G1, Corynebacterium group G2, Streptococcus mitis, Streptococcus sanguinis, Klebsiella pneumoniae, Pseudomonas aeruginosa, Burkholderia cepacia, Serratia marcescens, Haemophilus influenzae, Moraxella sp., Neisseria meningitidis, Neisseria gonorrhoeae, Salmonella typhimurium, Actinomyces israelii., Porphyromonas gingivalis., Prevotella melaninogenicus, Helicobacter pylori, Helicobacter felis, Enterococcus faecalis, Enterococcus faecium, Enterococcus gallinarum, Bacteroides fragilis, Bacteroides thetaiotaomicron, Fusobacterium nucleatum, Ruminococcus gnavus, or Campylobacter jejuni. Further non-limiting examples of bacteria include lactic acid bacteria including but not limited to Lactobacillus spp. and Bifidobacterium spp.; electrofuel bacterial strains including but not limited to Geobacter spp., Clostridium spp., or Ralstonia eutropha; or bacteria pathogenic on, for example, plants and mammals. In some embodiments, the bacterium is Pseudomonas aeruginosa. In some embodiments, the bacterium is Escherichia coli. In some embodiments, the bacterium is Clostridioides difficile. In some embodiments, the bacterium is Staphylococcus aureus. In some embodiments, the bacterium is Klebsiella pneumoniae. In some embodiments, the bacterium is Enterococcus faecalis. In some embodiments, the bacterium is Enterococcus faecium. In some embodiments, the bacterium is Bacteroides fragilis. In some embodiments, the bacterium is Bacteroides thetaiotaomicron. In some embodiments, the bacterium is Fusobacterium nucleatum. In some embodiments, the bacterium is Enterococcus gallinarum. In some embodiments, the bacterium is Ruminococcus gnavus. In some embodiments, the bacterium is Acinetobacter baumannii. In some embodiments, the bacterium is Mycobacterium tuberculosis. In some embodiments, the bacterium is Streptococcus pneumoniae. In some embodiments, the bacterium is Haemophilus influenzae. In some embodiments, the bacterium is Neisseria gonorrhoeae.

In some embodiments, the target bacterium causes an infection or disease. In some embodiments, the infection or disease is acute or chronic. In some embodiments, the infection or disease is localized or systemic. In some embodiments, infection or disease is idiopathic. In some embodiments, the infection or disease is acquired through means including, but not limited to, respiratory inhalation, ingestion, skin and wound infections, blood stream infections, middle-ear infections, gastrointestinal tract infections, peritoneal membrane infections, urinary tract infections, urogenital tract infections, oral soft tissue infections, intra-abdominal infections, epidermal or mucosal absorption, eye infections (including contact lens contamination), endocarditis, infections in cystic fibrosis, infections of indwelling medical devices such as joint prostheses, dental implants, catheters and cardiac implants, sexual contact, and/or hospital-acquired and ventilator-associated bacterial pneumonias. In some embodiments, the target bacterium causes urinary tract infection. In some embodiments, the E. coli causes urinary tract infection. In some embodiments, the target bacterium causes and/or exacerbates an inflammatory disease. In some embodiments, the target bacterium causes and/or exacerbates an autoimmune disease. In some embodiments, the target bacterium causes and/or exacerbates inflammatory bowel disease (IBD). In some embodiments, the E. coli causes inflammatory bowel disease (IBD). In some embodiments, the target bacterium causes and/or exacerbates psoriasis. In some embodiments, the target bacterium causes and/or exacerbates psoriatic arthritis (PA). In some embodiments, the target bacterium causes and/or exacerbates rheumatoid arthritis (RA). In some embodiments, the target bacterium causes and/or exacerbates systemic lupus erythematosus (SLE). In some embodiments, the target bacterium causes and/or exacerbates multiple sclerosis (MS). In some embodiments, the target bacterium causes and/or exacerbates Graves' disease. In some embodiments, the target bacterium causes and/or exacerbates Hashimoto's thyroiditis. In some embodiments, the target bacterium causes and/or exacerbates Myasthenia gravis. In some embodiments, the target bacterium causes and/or exacerbates vasculitis. In some embodiments, the target bacterium causes and/or exacerbates cancer. In some embodiments, the target bacterium causes and/or exacerbates cancer progression. In some embodiments, the target bacterium causes and/or exacerbates cancer metastasis. In some embodiments, the target bacterium causes and/or exacerbates resistance to cancer therapy. In some embodiments, the therapy used to address cancer includes, but is not limited to, chemotherapy, immunotherapy, hormone therapy, targeted drug therapy, and/or radiation therapy. In some embodiments, the cancer develops in organs including, but not limited to the, anus, bladder, blood and blood components, bone, bone marrow, brain, breast, cervix uteri, colon and rectum, esophagus, kidney, larynx, lymphatic system, muscle (i.e., soft tissue), oral cavity and pharynx, ovary, pancreas, prostate, skin, small intestine, stomach, testis, thyroid, uterus, and/or vulva. In some embodiments, the target bacterium causes and/or exacerbates disorders of the central nervous system (CNS). In some embodiments, the target bacterium causes and/or exacerbates attention deficit/hyperactivity disorder (ADHD). In some embodiments, the target bacterium causes and/or exacerbates autism. In some embodiments, the target bacterium causes and/or exacerbates bipolar disorder. In some embodiments, the target bacterium causes and/or exacerbates major depressive disorder. In some embodiments, the target bacterium causes and/or exacerbates epilepsy. In some embodiments, the target bacterium causes and/or exacerbates neurodegenerative disorders including, but not limited to, Alzheimer's disease, Huntington's disease, and/or Parkinson's disease.

Cystic fibrosis and cystic fibrosis-associated bronchiectasis is associated with infection by Pseudomonas aeruginosa. See, e.g., P. Farrell, et al, Radiology, Vol. 252, No. 2, pp. 534-543 (2009). In some embodiments, one or more bacteriophage are administered to a patient with cystic fibrosis or cystic fibrosis-associated bronchiectasis. In some embodiments, a combination of two or more bacteriophage are administered to a patient with cystic fibrosis or cystic fibrosis-associated bronchiectasis. In some embodiments, administration of the bacteriophage to a patient with cystic fibrosis or cystic fibrosis-associated bronchiectasis results in a reduction in bacterial load in the patient. In some embodiments, the reduction in bacterial load results in a clinical improvement in the patient with cystic fibrosis or cystic fibrosis-associated bronchiectasis.

Non-cystic fibrosis bronchiectasis is associated with infection by Pseudomonas aeruginosa. See, e.g., R. Wilson, et al, Respiratory Medicine, Vol. 117, pp. 179-189 (2016). In some embodiments, one or more bacteriophage are administered to a patient with non-cystic fibrosis bronchiectasis. In some embodiments, a combination of two or more bacteriophage are administered to a patient with non-cystic fibrosis bronchiectasis. In some embodiments, administration of the bacteriophage to a patient with non-cystic fibrosis bronchiectasis results in a reduction in bacterial load in the patient. In some embodiments, the reduction in bacterial load results in a clinical improvement in the patient with non-cystic fibrosis bronchiectasis.

### Bacteriophage

In some embodiments, the bacteriophage is an obligate lytic bacteriophage. In some embodiments, the bacteriophage is a temperate bacteriophage with retained lysogeny genes. In some embodiments, the bacteriophage is a temperate bacteriophage with some lysogeny genes removed, replaced, or inactivated. In some embodiments, the bacteriophage is a temperate bacteriophage with a lysogeny gene removed, replaced, or inactivated, thereby rendering the bacteriophage lytic. In some embodiments, the bacteriophages include, but are not limited to, p2131, p2132, p2973, p4209, p1106, p1587, p1835, p2037, p2421, p2363, p1772, PB1, p004k, or p004ex. In some embodiments, the bacteriophage is p1772, p2131, p2132, p2973, p4209, p1106, p1587, p1835, p2037, p2421, p2363, or PB1, which target Pseudomonas ssp. In some embodiments, the bacteriophage is p004k, or p00ex, which target Escherichia ssp. In some embodiments, the bacteriophage targets Pseudomonas spp. In some embodiments, the bacteriophage targets Pseudomonas aeruginosa. In some embodiments, the bacteriophages include, but are not limited to, p004k, or p00ex. In some embodiments, the bacteriophage targets Escherichia spp. In some embodiments, the bacteriophage targets Escherichia coli. In some embodiments, the bacteriophage targets Staphylococcus spp. In some embodiments, the bacteriophage targets Staphylococcus aureus. In some embodiments, the bacteriophage targets Klebsiella spp. In some embodiments, the bacteriophage targets Klebsiella pneumoniae. In some embodiments, the bacteriophage targets Enterococcus spp. In some embodiments, the bacteriophage targets Enterococcus faecium. In some embodiments, the bacteriophage targets Enterococcus faecalis. In some embodiments, the bacteriophage targets Enterococcus gallinarum. In some embodiments, the bacteriophage targets Clostridioides spp. In some embodiments, the bacteriophage targets Clostridioides difficile. In some embodiments, the bacteriophage targets Bacteroides spp. In some embodiments, the bacteriophage targets Bacteroides fragilis. In some embodiments, the bacteriophage targets Bacteroides thetaiotaomicron. In some embodiments, the bacteriophage targets Fusobacterium spp. In some embodiments, the bacteriophage targets Fusobacterium nucleatum. In some embodiments, the bacteriophage targets Streptococcus spp. In some embodiments, the bacteriophage targets Streptococcus pneumoniae. In some embodiments, the bacteriophage targets Acinetobacter spp. In some embodiments, the bacteriophage targets Acinetobacter baumannii. In some embodiments, the bacteriophage targets Mycobacterium spp. In some embodiments, the bacteriophage targets Mycobacterium tuberculosis. In some embodiments, the bacteriophage targets Haemophilus spp. In some embodiments, the bacteriophage targets Haemophilus influenzae. In some embodiments, the bacteriophage targets Neisseria spp. In some embodiments, the bacteriophage targets Neisseria gonorrhoeae. In some embodiments, the bacteriophage targets Ruminococcus spp. In some embodiments, the bacteriophage targets Ruminococcus gnavus.

In some embodiments, bacteriophages of interest are obtained from environmental sources or from commercial research vendors. In some embodiments, obtained bacteriophages are screened for lytic activity against a library of bacteria and their associated strains. In some embodiments, the bacteriophages are screened against a library of bacteria and their associated strains for their ability to generate primary resistance in the screened bacteria.

### Insertion Sites

In some embodiments, the insertion of the nucleic acid sequence into a bacteriophage preserves the lytic activity of the bacteriophage. In some embodiments, the nucleic acid sequence is inserted into the bacteriophage genome. In some embodiments, the nucleic acid sequence is inserted into the bacteriophage genome at a transcription terminator site at the end of an operon of interest. In some embodiments, the nucleic acid sequence is inserted into the bacteriophage genome as a replacement for one or more removed non-essential genes. In some embodiments, the nucleic acid sequence is inserted into the bacteriophage genome as a replacement for one or more removed lysogenic genes. In some embodiments, the replacement of non-essential and/or lysogenic genes with the nucleic acid sequence does not affect the lytic activity of the bacteriophage. In some embodiments, the replacement of non-essential and/or lysogenic genes with the nucleic acid sequence preserves the lytic activity of the bacteriophage. In some embodiments, the replacement of non-essential and/or lysogenic genes with the nucleic acid sequence enhances the lytic activity of the bacteriophage. In some embodiments, the replacement of non-essential and/or lysogenic genes with the nucleic acid sequence renders a lysogenic bacteriophage lytic.

In some embodiments, the nucleic acid sequence is introduced into the bacteriophage genome at a first location while one or more non-essential and/or lysogenic genes are separately removed and/or inactivated from the bacteriophage genome at a separate location. In some embodiments, the nucleic acid sequence is introduced into the bacteriophage at a first location while one or more non-essential and/or lysogenic genes are separately removed and/or inactivated from the bacteriophage genome at multiple separate locations. In some embodiments, the removal and/or inactivation of one or more non-essential and/or lysogenic genes does not affect the lytic activity of the bacteriophage. In some embodiments, the removal and/or inactivation of one or more non-essential and/or lysogenic genes preserves the lytic activity of the bacteriophage. In some embodiments, the removal of one or more non-essential and/or lysogenic genes renders a lysogenic bacteriophage into a lytic bacteriophage.

In some embodiments, the bacteriophage is a temperate bacteriophage which has been rendered lytic by any of the aforementioned means. In some embodiments, a temperate bacteriophage is rendered lytic by the removal, replacement, or inactivation of one or more lysogenic genes. In some embodiments, the lytic activity of the bacteriophage is due to the removal, replacement, or inactivation of at least one lysogeny gene. In some embodiments, the lysogenic gene plays a role in the maintenance of lysogenic cycle in the bacteriophage. In some embodiments, the lysogenic gene plays a role in establishing the lysogenic cycle in the bacteriophage. In some embodiments, the lysogenic gene plays a role in both establishing the lysogenic cycle and in the maintenance of the lysogenic cycle in the bacteriophage. In some embodiments, the lysogenic gene is a repressor gene. In some embodiments, the lysogenic gene is cI repressor gene. In some embodiments, the lysogenic gene is an activator gene. In some embodiments, the lysogenic gene is cII gene. In some embodiments, the lysogenic gene is lexA gene. In some embodiments, the lysogenic gene is int (integrase) gene. In some embodiments, two or more lysogeny genes are removed, replaced, or inactivated to cause arrest of a bacteriophage lysogeny cycle and/or induction of a lytic cycle. In some embodiments, a temperate bacteriophage is rendered lytic by the insertion of one or more lytic genes. In some embodiments, a temperate bacteriophage is rendered lytic by the insertion of one or more genes that contribute to the induction of a lytic cycle. In some embodiments, a temperate bacteriophage is rendered lytic by altering the expression of one or more genes that contribute to the induction of a lytic cycle. In some embodiments, a temperate bacteriophage phenotypically changes from a lysogenic bacteriophage to a lytic bacteriophage. In some embodiments, a temperate bacteriophage is rendered lytic by environmental alterations. In some embodiments, environmental alterations include, but are not limited to, alterations in temperature, pH, or nutrients, exposure to antibiotics, hydrogen peroxide, foreign DNA, or DNA damaging agents, presence of organic carbon, and presence of heavy metal (e.g. in the form of chromium (VI). In some embodiments, a temperate bacteriophage that is rendered lytic is prevented from reverting to lysogenic state. In some embodiments, a temperate bacteriophage that is rendered lytic is prevented from reverting back to lysogenic state by way the self-targeting activity of the first introduced CRISPR array. In some embodiments, a temperate bacteriophage that is rendered lytic is prevented from reverting back to lysogenic state by way of introducing an additional CRISPR array. In some embodiments, the bacteriophage does not confer any new properties onto the target bacterium beyond cellular death cause by lytic activity of the bacteriophage and/or the activity of the first or second CRISPR array.

In some embodiments, the replacement, removal, inactivation, or any combination thereof, of one or more non-essential and/or lysogenic genes is achieved by chemical, biochemical, and/or any suitable method. In some embodiments, the insertion of one or more lytic genes is achieved by any suitable chemical, biochemical, and/or physical method by homologous recombination.

### Non-Essential Gene

In some embodiments, the non-essential gene to be removed and/or replaced from the bacteriophage is a gene that is non-essential for the survival of the bacteriophage. In some embodiments, the non-essential gene to be removed and/or replaced from the bacteriophage is a gene that is non-essential for the induction and/or maintenance of lytic cycle. In some embodiments, the non-essential gene to be removed and/or replaced from the bacteriophage is the hoc gene from a T4 E. coli bacteriophage. In some embodiments, the non-essential gene to be removed and/or replaced include gp0.7, gp4.3, gp4.5, gp4.7, or any combination thereof from a T7 E. coli bacteriophage. In some embodiments, the non-essential gene to be removed and/or replaced is gp0.6, gp0.65, gp0.7, gp4.3, gp4.5, or any combination thereof from a T7m E. coli bacteriophage.

### Antimicrobial Agents and Peptides

In some embodiments, a bacteriophage disclosed herein is further genetically modified to express an antibacterial peptide, a functional fragment of an antibacterial peptide or a lytic gene. In some embodiments, a bacteriophage disclosed herein express at least one antimicrobial agent or peptide disclosed herein. In some embodiments, a bacteriophage disclosed herein comprises a nucleic acid sequence that encodes an enzybiotic where the protein product of the nucleic acid sequence targets phage resistant bacteria. In some embodiments, the bacteriophage comprises nucleic acids which encode enzymes which assist in breaking down or degrading biofilm matrix. In some embodiments, a bacteriophage disclosed herein comprises nucleic acids encoding Dispersin D aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, xylanase or lyase. In some embodiments, the enzyme is selected from the group consisting of cellulases, such as glycosyl hydroxylase family of cellulases, such as glycosyl hydroxylase 5 family of enzymes also called cellulase A; polyglucosamine (PGA) depolymerases; and colonic acid depolymerases, such as 1,4-L-fucodise hydrolase, colanic acid, depolymerazing alginase, DNase I, or combinations thereof. In some embodiments, a bacteriophage disclosed herein secretes an enzyme disclosed herein.

In some embodiments, an antimicrobial agent or peptide is expressed and/or secreted by a bacteriophage disclosed herein. In some embodiments, a bacteriophage disclosed herein secretes and expresses an antibiotic such as ampicillin, penicillin, penicillin derivatives, cephalosporins, monobactams, carbapenems, ofloxacin, ciproflaxacin, levofloxacin, gatifloxacin, norfloxacin, lomefloxacin, trovafloxacin, moxifloxacin, sparfloxacin, gemifloxacin, pazufloxacin or any antibiotic disclosed herein. In some embodiments, a bacteriophage disclosed herein comprises a nucleic acid sequence encoding an antibacterial peptide, expresses an antibacterial peptide, or secretes a peptide that aids or enhances killing of a target bacterium. In some embodiments, a bacteriophage disclosed herein comprises a nucleic acid sequence encoding a peptide, a nucleic acid sequence encoding an antibacterial peptide, expresses an antibacterial peptide, or secretes a peptide that aids or enhances the activity of the first and/or the second Type I CRISPR-Cas system.

### Methods of Use

Disclosed herein, in certain embodiments, are methods of killing a target bacterium comprising introducing into a target bacterium any of the bacteriophages disclosed herein.

Further disclosed herein, in certain embodiments, are methods of modifying a mixed population of bacterial cells having a first bacterial species that comprises a target nucleotide sequence in the essential gene and a second bacterial species that does not comprise a target nucleotide sequence in the essential gene, the method comprising introducing into the mixed population of bacterial cells any of the bacteriophages disclosed herein.

Also disclosed herein, in certain embodiments, are methods of treating a disease in an individual in need thereof, the method comprising administering to the individual any of the bacteriophages disclosed herein.

In some embodiments, the target bacterium is killed solely by lytic activity of the bacteriophage. In some embodiments, the target bacterium is killed solely by activity of the CRISPR-Cas system. In some embodiments, the target bacterium is killed by the processing of the CRISPR array by a CRISPR-Cas system to produce a processed crRNA capable of directing CRISPR-Cas based endonuclease activity and/or cleavage at the target nucleotide sequence in the target gene of the bacterium.

In some embodiments, the target bacterium is killed by lytic activity of the bacteriophage in combination with activity of the Type I CRISPR-Cas system. In some embodiments, the target bacterium is killed by the activity of the Type I CRISPR-Cas system, independently of the lytic activity of the bacteriophage. In some embodiments, the activity of the Type I CRISPR-Cas system supplements or enhances the lytic activity of the bacteriophage.

In some embodiments, the lytic activity of the bacteriophage and the activity of the Type I CRISPR-Cas system is synergistic. In some embodiments, the lytic activity of the bacteriophage is modulated by a concentration of the bacteriophage. In some embodiments, the activity of the Type I CRISPR-Cas system is modulated by a concentration of the bacteriophage.

In some embodiments, the synergistic killing of the bacterium is modulated to favor killing by the lytic activity of the bacteriophage over the activity of the first CRISPR-Cas system by increasing the concentration of bacteriophage administered to the bacterium. In some embodiments, the synergistic killing of the bacterium is modulated to disfavor killing by the lytic activity of the bacteriophage over the activity of the CRISPR-Cas system by decreasing the concentration of bacteriophage administered to the bacterium. In some embodiments, at low concentrations, lytic replication allows for amplification and killing of the target bacteria. In some embodiments, at high concentrations, amplification of a phage is not required. In some embodiments, the synergistic killing of the bacterium is modulated to favor killing by the activity of the CRISPR-Cas system over the lytic activity of the bacteriophage by altering the number, the length, the composition, the identity, or any combination thereof, of the spacers so as to increase the lethality of the CRISPR array. In some embodiments, the synergistic killing of the bacterium is modulated to disfavor killing by the activity of the CRISPR-Cas system over the lytic activity of the bacteriophage by altering the number, the length, the composition, the identity, or any combination thereof, of the spacers so as to decrease the lethality of the CRISPR array.

### Administration Routes and Dosage

Dose and duration of the administration of a composition disclosed herein will depend on a variety of factors, including the subject's age, subject's weight, and tolerance of the phage. In some embodiments, a bacteriophage disclosed herein is administered to patients intra-arterially, intravenously, intraurethrally, intramuscularly, orally, subcutaneously, by inhalation, or any combination thereof. In some embodiments, a bacteriophage disclosed herein is administered to patients by oral administration.

In some embodiments, a dose of phage between 10³ and 10²⁰ PFU is given. In some embodiments, a dose of phage between 10³ and 10¹⁰ PFU is given. In some embodiments, a dose of phage between 10⁶ and 10²⁰ PFU is given. In some embodiments, a dose of phage between 10⁶ and 10¹⁰ PFU is given. For example, in some embodiments, the bacteriophage is present in a composition in an amount between 10³ and 10¹¹ PFU. In some embodiments, the bacteriophage is present in a composition in an amount about 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², 10²³, 10²⁴ PFU or more. In some embodiments, the bacteriophage is present in a composition in an amount of less than 10¹ PFU. In some embodiments, the bacteriophage is present in a composition in an amount between 10¹ and 10⁸, 10⁴ and 10⁹, 10⁵ and 10¹⁰, or 10⁷ and 10¹¹PFU.

In some embodiments, a bacteriophage or a mixture is administered to a subject in need thereof 1, 2, 3, 4, 5, 6, 7, 8, 9, 10, 11, 12, 13, 14, 15, 16, 17, 18, 19, 20, 21, 22, 23, or 24 times a day. In some embodiments, a bacteriophage or a mixture is administered to a subject in need thereof at least 1, 2, 3, 4, 5, 6, 7, 8, 9, 10, 11, 12, 13, 14, 15, 16, 17, 18, 19, 20, or 21 times a week. In some embodiments, a bacteriophage or a mixture is administered to a subject in need thereof at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month. In some embodiments, a bacteriophage or a mixture is administered to a subject in need thereof every 2, 4, 6, 8, 10, 12, 14, 18, 20, 22, or 24 hours.

In some embodiments, the compositions (bacteriophage) disclosed herein are administered before, during, or after the occurrence of a disease or condition. In some embodiment, the timing of administering the composition containing the bacteriophage varies. In some embodiments, the pharmaceutical compositions are used as a prophylactic and are administered continuously to subjects with a propensity to conditions or diseases in order to prevent the occurrence of the disease or condition. In some embodiments, pharmaceutical compositions are administered to a subject during or as soon as possible after the onset of the symptoms. In some embodiments, the administration of the compositions is initiated within the first 48 hours of the onset of the symptoms, within the first 24 hours of the onset of the symptoms, within the first 6 hours of the onset of the symptoms, or within 3 hours of the onset of the symptoms. In some embodiments, the initial administration of the composition is via any route practical, such as by any route described herein using any formulation described herein. In some embodiments, the compositions is administered as soon as is practicable after the onset of a disease or condition is detected or suspected, and for a length of time necessary for the treatment of the disease, such as, for example, from about 1 month to about 3 months. In some embodiments, the length of treatment will vary for each subject.

### Bacterial Infections

Disclosed herein, in certain embodiments, are methods of treating bacterial infections. In some embodiments, the bacteriophages disclosed herein treat or prevent diseases or conditions mediated or caused by bacteria as disclosed herein in a human or animal subject. In some embodiments, the bacteriophages disclosed herein treat or prevent diseases or conditions caused or exacerbated by bacteria as disclosed herein in a human or animal subject. Such bacteria are typically in contact with tissue of the subject including: gut, oral cavity, lung, armpit, ocular, vaginal, anal, ear, nose or throat tissue. In some embodiments, a bacterial infection is treated by modulating the activity of the bacteria and/or by directly killing of the bacteria.

In some embodiments, non-limiting examples of target bacteria includes Escherichia spp., Salmonella spp., Bacillus spp., Corynebacterium Clostridium spp., Clostridium spp., Pseudomonas spp., Clostridium spp., Lactococcus spp., Acinetobacter spp., Mycobacterium spp., Myxococcus spp., Staphylococcus spp., Streptococcus spp., or cyanobacteria. In some embodiments, non-limiting examples of bacteria include Escherichia coli, Salmonella enterica, Bacillus subtilis, Clostridium acetobutylicum, Clostridium ljungdahlii, Clostridium difficile, Acinetobacter baumannii, Mycobacterium tuberculosis, Myxococcus xanthus, Staphylococcus aureus, Streptococcus pyogenes, or cyanobacteria. In some embodiments, non-limiting examples of bacteria include Staphylococcus aureus, methicillin resistant Staphylococcus aureus, Streptococcus pneumonia, carbapenem-resistant Enterobacteriaceae, Staphylococcus epidermidis, Staphylococcus salivarius, Corynebacterium minutissium, Corynebacterium pseudodiphtherias, Corynebacterium stratium, Corynebacterium group G1, Corynebacterium group G2, Streptococcus pneumonia, Streptococcus mitis, Streptococcus sanguis, Escherichia coli, Klebsiella pneumoniae, Pseudomonas aeruginosa, Burkholderia cepacia, Serratia marcescens, Haemophilus influenzae, Moraxella sp., Neisseria meningitidis, Neisseria gonorrhoeae, Salmonella typhimurium, Actinomyces spp., Porphyromonas spp., Prevotella melaninogenicus, Helicobacter pylori, Helicobacter felis, or Campylobacter jejuni. Further non-limiting examples of bacteria include lactic acid bacteria including but not limited to Lactobacillus spp. and Bifidobacterium spp.; electrofuel bacterial strains including but not limited to Geobacter spp., Clostridium spp., or Ralstonia eutropha; or bacteria pathogenic on, for example, plants and mammals. In some embodiments, the bacterium is Escherichia coli. In some embodiments, the bacterium is Clostridium difficile. In some embodiments, the bacterium is Pseudomonas aeruginosa.

In some embodiments, one or more target bacteria present in a bacterial population are pathogenic. In some embodiments, the pathogenic bacteria are uropathogenic. In some embodiments, the pathogenic bacterium is uropathogenic E. coli (UPEC). In some embodiments, the pathogenic bacteria are diarrheagenic. In some embodiments, the pathogenic bacteria are diarrheagenic E. coli (DEC). In some embodiments, the pathogenic bacteria are Shiga-toxin producing. In some embodiments, the pathogenic bacterium is Shiga-toxin producing E. coli (STEC). In some embodiments, the pathogenic bacteria are Shiga-toxin producing. In some embodiments, the pathogenic bacterium is Shiga-toxin producing E. coli (STEC). In some embodiments, the pathogenic bacterium is Shiga-toxin producing E. coli (STEC). In some embodiments, the pathogenic bacteria are various O-antigen:H-antigen serotype E. coll. In some embodiments, the pathogenic bacteria are enteropathogenic. In some embodiments, the pathogenic bacterium is enteropathogenic E. coli (EPEC).

In some embodiments, the pathogenic bacteria are various strains of C. difficile including: CD043, CD05, CD073, CD093, CD180, CD106, CD128, CD199, CD111, CD108 CD25, CD148, CD154, FOBT195, CD03, CD038, CD112, CD196, CD105, UK1, UK6, BI-9, CD041, CD042, CD046, CD19, or R20291.

In some embodiments, the bacteriophages disclosed herein are used to treat an infection, a disease, or a condition, in the gastrointestinal tract of a subject. In some embodiments, the bacteriophages are used to modulate and/or kill target bacteria within the microbiome or gut flora of a subject. In some embodiments, the bacteriophages are used to selectively modulate and/or kill one or more target bacteria from a plurality of bacteria within the microbiome or gut flora of a subject. In some embodiments, the bacteriophages are used to selectively modulate and/or kill one or more target enteropathogenic bacteria from a plurality of bacteria within the microbiome or gut flora of a subject. In some embodiments, the target enteropathogenic bacterium is enteropathogenic E. coli (EPEC). In some embodiments, the bacteriophages are used to selectively modulate and/or kill one or more target diarrheagenic bacteria from a plurality of bacteria within the microbiome or gut flora of a subject. In some embodiments, the target diarrheagenic bacterium is diarrheagenic E. coli (DEC). In some embodiments, the bacteriophages are used to selectively modulate and/or kill one or more target Shiga-toxin producing bacteria from a plurality of bacteria within the microbiome or gut flora of a subject. In some embodiments, the target Shiga-toxin producing bacterium is Shiga-toxin producing E. coli (STEC).

In some embodiments, the bacteriophages are used to selectively modulate and/or kill one or more target enteropathogenic C. difficile bacteria strains within the microbiome or gut flora of a subject including: CD043, CD05, CD073, CD093, CD180, CD106, CD128, CD199, CD111, CD108, CD25, CD148, CD154, FOBT195, CD03, CD038, CD112, CD196, CD105, UK1, UK6, BI-9, CD041, CD042, CD046, CD19, or R20291.

In some embodiments, the bacteriophages disclosed herein are used to treat an infection, a disease, or a condition, in the urinary tract of a subject. In some embodiments, the bacteriophages are used to modulate and/or kill target bacteria within the urinary tract flora of a subject. The urinary tract flora includes, but is not limited, to Staphylococcus epidermidis, Enterococcus faecalis, and some alpha-hemolytic Streptococci. In some embodiments, the bacteriophages are used to selectively modulate and/or kill one or more target uropathogenic bacteria from a plurality of bacteria within the urinary tract flora of a subject. In some embodiments, the target bacterium is uropathogenic E. coli (UPEC).

In some embodiments, the bacteriophages disclosed herein are used to treat an infection, a disease, or a condition, on the skin of a subject. In some embodiments, the bacteriophages are used to modulate and/or kill target bacteria on the skin of a subject.

In some embodiments, the bacteriophages disclosed herein are used to treat an infection, a disease, or a condition, on a mucosal membrane of a subject. In some embodiments, the bacteriophages are used to modulate and/or kill target bacteria on the mucosal membrane of a subject.

In some embodiments, the pathogenic bacteria are antibiotic resistant. In one embodiment, the pathogenic bacterium is methicillin-resistant Staphylococcus aureus (MRSA).

In some embodiments, the one or more target bacteria present in the bacterial population form a biofilm. In some embodiments, the biofilm comprises pathogenic bacteria. In some embodiments, the bacteriophage disclosed herein is used to treat a biofilm.

In some embodiments, non-limiting examples of target bacteria includes Escherichia spp., Salmonella spp., Bacillus spp., Corynebacterium Clostridium spp., Clostridium spp., Pseudomonas spp., Clostridium spp., Lactococcus spp., Acinetobacter spp., Mycobacterium spp., Myxococcus spp., Staphylococcus spp., Streptococcus spp., or cyanobacteria. In some embodiments, non-limiting examples of bacteria include Escherichia coli, Salmonella enterica, Bacillus subtilis, Clostridium acetobutylicum, Clostridium ljungdahlii, Clostridium difficile, Acinetobacter baumannii, Mycobacterium tuberculosis, Myxococcus xanthus, Staphylococcus aureus, Streptococcus pyogenes, or cyanobacteria. In some embodiments, non-limiting examples of bacteria include Staphylococcus aureus, methicillin resistant Staphylococcus aureus, Streptococcus pneumonia, carbapenem-resistant Enterobacteriaceae, Staphylococcus epidermidis, Staphylococcus salivarius, Corynebacterium minutissium, Corynebacterium pseudodiphtherias, Corynebacterium stratium, Corynebacterium group G1, Corynebacterium group G2, Streptococcus pneumonia, Streptococcus mitis, Streptococcus sanguis, Escherichia coli, Klebsiella pneumoniae, Pseudomonas aeruginosa, Burkholderia cepacia, Serratia marcescens, Haemophilus influenzae, Moraxella sp., Neisseria meningitidis, Neisseria gonorrhoeae, Salmonella typhimurium, Actinomyces spp., Porphyromonas spp., Prevotella melaninogenicus, Helicobacter pylori, Helicobacter felis, or Campylobacter jejuni. Further non-limiting examples of bacteria include lactic acid bacteria including but not limited to Lactobacillus spp. and Bifidobacterium spp.; electrofuel bacterial strains including but not limited to Geobacter spp., Clostridium spp., or Ralstonia eutropha; or bacteria pathogenic on, for example, plants and mammals. In some embodiments, the bacterium is Escherichia coli. In some embodiments, the bacterium is Clostridium difficile.

In some embodiments, the bacteriophage treats acne and other related skin infections.

In some embodiments, a target bacterium is a multiple drug resistant (MDR) bacteria strain. An MDR strain is a bacteria strain that is resistant to at least one antibiotic. In some embodiments, a bacteria strain is resistant to an antibiotic class such as a cephalosporin, a fluoroquinolone, a carbapenem, a colistin, an aminoglycoside, vancomycin, streptomycin, and methicillin. In some embodiments, a bacteria strain is resistant to an antibiotic such as a Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Linezolid, a Streptogramin, Tigecycline, Daptomycin, or any combination thereof. Examples of MDR strains include: Vancomycin-Resistant Enterococci (VRE), Methicillin-Resistant Staphylococcus aureus (MRSA), Extended-spectrum β-lactamase (ESBLs) producing Gram-negative bacteria, Klebsiella pneumoniae carbapenemase (KPC) producing Gram-negatives, and Multidrug-Resistant gram negative rods (MDR GNR) MDRGN bacteria such as Enterobacter species E. coli, Klebsiella pneumoniae, Acinetobacter baumannii, or Pseudomonas aeruginosa.

In some embodiments the target bacterium is Klebsiella pneumoniae. In some embodiments, the target bacterium is Staphylococcus aureus. In some embodiments, the target bacterium is Enterococci. In some embodiments, the target bacterium is Acinetobacter. In some embodiments, the target bacterium is Pseudomonas. In some embodiments, the target bacterium is Enterobacter. In some embodiments, the target bacterium is Clostridium difficile. In some embodiments, the target bacterium is E. coli. In some embodiments, the target bacterium is Clostridium bolteae. In some embodiments, the methods and compositions disclosed herein are for use in veterinary and medical applications as well as research applications.

### Microbiome

“Microbiome”, “microbiota”, and “microbial habitat” are used interchangeably hereinafter and refer to the ecological community of microorganisms that live on or in a subject's bodily surfaces, cavities, and fluids. Non-limiting examples of habitats of microbiome include: gut, colon, skin, skin surfaces, skin pores, vaginal cavity, umbilical regions, conjunctival regions, intestinal regions, stomach, nasal cavities and passages, gastrointestinal tract, urogenital tracts, saliva, mucus, and feces. In some embodiments, the microbiome comprises microbial material including, but not limited to, bacteria, archaea, protists, fungi, and viruses. In some embodiments, the microbial material comprises a gram-negative bacterium. In some embodiments, the microbial material comprises a gram-positive bacterium. In some embodiments, the microbial material comprises Proteobacteria, Actinobacteria, Bacteroidetes, or Firmicutes.

In some embodiments, the bacteriophages as disclosed herein are used to modulate or kill target bacteria within the microbiome of a subject. In some embodiments, the bacteriophages are used to modulate and/or kill target bacteria within the microbiome by the CRISPR-Cas system, lytic activity, or a combination thereof. In some embodiments, the bacteriophages are used to modulate and/or kill target bacteria within the microbiome of a subject. In some embodiments, the bacteriophages are used to selectively modulate and/or kill one or more target bacteria from a plurality of bacteria within the microbiome of a subject. In some embodiments, the target bacterium is E. coli. In some embodiments, the E. coli is a multidrug-resistant (MDR) strain. In some embodiments, the E. coli is an extended spectrum beta-lactamase (ESBL) strain. In some embodiments, the E. coli is a carbapenem-resistant strain. In some embodiments, the E. coli is a non-multidrug-resistant (non-MDR) strain. In some embodiments, the E. coli is a non-carbapenem-resistant strain. In some embodiments, the pathogenic bacteria are uropathogenic. In some embodiments, the pathogenic bacterium is uropathogenic E. coli (UPEC). In some embodiments, the pathogenic bacteria are diarrheagenic. In some embodiments, the pathogenic bacteria are diarrheagenic E. coli (DEC). In some embodiments, the pathogenic bacteria are Shiga-toxin producing. In some embodiments, the pathogenic bacterium is Shiga-toxin producing E. coli (STEC). In some embodiments, the pathogenic bacteria are various O-antigen:H-antigen serotype E. coli. In some embodiments, the pathogenic bacteria are enteropathogenic. In some embodiments, the pathogenic bacterium is enteropathogenic E. coli (EPEC).

In some embodiments, the bacteriophages are used to modulate or kill target single or plurality of bacteria within the microbiome or gut flora of the gastrointestinal tract of a subject. Modification (e.g., dysbiosis) of the microbiome or gut flora increases the risk for health conditions such as diabetes, mental disorders, ulcerative colitis, colorectal cancer, autoimmune disorders, obesity, diabetes, diseases of the central nervous system and inflammatory bowel disease. An exemplary bacteria associated with diseases and conditions of gastrointestinal tract and are being modulated or killed by the bacteriophages include strains, sub-strains, and enterotypes of E. coli.

In some embodiments, the bacteriophages are used to selectively modulate and/or kill one or more target enteropathogenic C. difficile bacteria strains within the microbiome of a subject including: CD043, CD05, CD073, CD093, CD180, CD106, CD128, CD199, CD111, CD108, CD25, CD148, CD154, FOBT195, CD03, CD038, CD112, CD196, CD105, UK1, UK6, BI-9, CD041, CD042, CD046, CD19, or R20291.

In some embodiments, the bacteriophages are used to modulate or kill target single or plurality of bacteria within the microbiome or gut flora of the gastrointestinal tract of a subject. Modification (e.g., dysbiosis) of the microbiome or gut flora increases the risk for health conditions such as diabetes, mental disorders, ulcerative colitis, colorectal cancer, autoimmune disorders, obesity, diabetes, diseases of the central nervous system and inflammatory bowel disease. An exemplary list of the bacteria associated with diseases and conditions of gastrointestinal tract and are being modulated or killed by the bacteriophages include strains, sub-strains, and enterotypes of enterobacteriaceae, pasteurellaceae, fusobacteriaceae, neisseriaceae, veillonellaceae, gemellaceae, bacteriodales, clostridiales, erysipelotrichaceae, bifidobacteriaceae Bacteroides, Faecalibacterium, Roseburia, Blautia, Ruminococcus, Coprococcus, Streptococcus, Dorea, Blautia, Ruminococcus, Lactobacillus, Enterococcus, Streptococcus, Escherichia coli, Fusobacterium nucleatum, Haemophilus parainfluenzae (pasteurellaceae), Veillonella parvula, Eikenella corrodens (neisseriaceae), Gemella moribillum, Bacteroides vulgatus, Bacteroides caccae, Bifidobacterium bifidum, Bifidobacterium longum, Bifidobacterium adolescentis, Bifidobacterium dentum, Blautia hansenii, Ruminococcus gnavus, Clostridium nexile, Faecalibacterium prausnitzii, Ruminoccus torques, Clostridium bolteae, Eubacterium rectale, Roseburia intestinalis, Coprococcus comes, Actinomyces, Lactococcus, Roseburia, Streptococcus, Blautia, Dialister, Desulfovibrio, Escherichia, Lactobacillus, Coprococcus, Clostridium, Bifidobacterium, Klebsiella, Granulicatella, Eubacterium, Anaerostipes, Parabacteroides, Coprobacillus, Gordonibacter, Collinsella, Bacteroides, Faecalibacterium, Anaerotruncus, Alistipes, Haemophilus, Anaerococcus, Veillonella, Arevotella, Akkermansia, Bilophila, Sutterella, Eggerthella, Holdemania, Gemella, Peptoniphilus, Rothia, Enterococcus, Pediococcus, Citrobacter, Odoribacter, Enterobacteria, Fusobacterium, and Proteus.

In some embodiments, a bacteriophage disclosed herein is administered to a subject to promote a healthy microbiome. In some embodiments, a bacteriophage disclosed herein is administered to a subject to restore a subject's microbiome to a microbiome composition that promotes health. In some embodiments, a composition comprising a bacteriophage disclosed herein comprises a prebiotic or a third agent. In some embodiment, microbiome related disease or disorder is treated by a bacteriophage disclosed herein.

### Environmental Therapy

In some embodiments, bacteriophages disclosed herein are further used for food and agriculture sanitation (including meats, fruits and vegetable sanitation), hospital sanitation, home sanitation, vehicle and equipment sanitation, industrial sanitation, etc. In some embodiments, bacteriophages disclosed herein are used for the removal of antibiotic-resistant or other undesirable pathogens from medical, veterinary, animal husbandry, or any additional environments bacteria are passed to humans or animals.

Environmental applications of phage in health care institutions are for equipment such as endoscopes and environments such as ICUs which are potential sources of nosocomial infection due to pathogens that are difficult or impossible to disinfect. In some embodiments, a phage disclosed herein is used to treat equipment or environments inhabited by bacterial genera which become resistant to commonly used disinfectants. In some embodiments, phage compositions disclosed herein are used to disinfect inanimate objects. In some embodiments, an environment disclosed herein is sprayed, painted, or poured onto with aqueous solutions with phage titers. In some embodiment a solution described herein comprises between 10¹-10²⁰ plaque forming units (PFU)/ml. In some embodiments, a bacteriophage disclosed herein is applied by aerosolizing agents that include dry dispersants to facilitate distribution of the bacteriophage into the environment. In some embodiments, objects are immersed in a solution containing bacteriophage disclosed herein.

### Sanitation

In some embodiments, bacteriophages disclosed herein are used as sanitation agents in a variety of fields. Although the terms “phage” or “bacteriophage” may be used, it should be noted that, where appropriate, this term should be broadly construed to include a single bacteriophage, multiple bacteriophages, such as a bacteriophage mixtures and mixtures of a bacteriophage with an agent, such as a disinfectant, a detergent, a surfactant, water, etc.

In some embodiments, bacteriophages are used to sanitize hospital facilities, including operating rooms, patient rooms, waiting rooms, lab rooms, or other miscellaneous hospital equipment. In some embodiments, this equipment includes electrocardiographs, respirators, cardiovascular assist devices, intraaortic balloon pumps, infusion devices, other patient care devices, televisions, monitors, remote controls, telephones, beds, etc. In some situations, the bacteriophage is applied through an aerosol canister. In some embodiments, bacteriophage is applied by wiping the phage on the object with a transfer vehicle.

In some embodiments, a bacteriophage described herein is used in conjunction with patient care devices. In some embodiment, bacteriophage is used in conjunction with a conventional ventilator or respiratory therapy device to clean the internal and external surfaces between patients. Examples of ventilators include devices to support ventilation during surgery, devices to support ventilation of incapacitated patients, and similar equipment. In some embodiments, the conventional therapy includes automatic or motorized devices, or manual bag-type devices such as are commonly found in emergency rooms and ambulances. In some embodiments, respiratory therapy includes inhalers to introduce medications such as bronchodilators as commonly used with chronic obstructive pulmonary disease or asthma, or devices to maintain airway patency such as continuous positive airway pressure devices.

In some embodiment, a bacteriophage described herein is used to cleanse surfaces and treat colonized people in an area where highly-contagious bacterial diseases, such as meningitis or enteric infections are present.

In some embodiments, water supplies are treated with a composition disclosed herein. In some embodiments, bacteriophage disclosed herein is used to treat contaminated water, water found in cisterns, wells, reservoirs, holding tanks, aqueducts, conduits, and similar water distribution devices. In some embodiments, the bacteriophage is applied to industrial holding tanks where water, oil, cooling fluids, and other liquids accumulate in collection pools. In some embodiments, a bacteriophage disclosed herein is periodically introduced to the industrial holding tanks in order to reduce bacterial growth.

In some embodiments, bacteriophages disclosed herein are used to sanitize a living area, such as a house, apartment, condominium, dormitory, or any living area. In some embodiments, the bacteriophage is used to sanitize public areas, such as theaters, concert halls, museums, train stations, airports, pet areas, such as pet beds, or litter boxes. In this capacity, the bacteriophage is dispensed from conventional devices, including pump sprayers, aerosol containers, squirt bottles, pre-moistened towelettes, etc, applied directly to (e.g., sprayed onto) the area to be sanitized, or be transferred to the area via a transfer vehicle, such as a towel, sponge, etc. In some embodiments, a phage disclosed herein is applied to various rooms of a house, including the kitchen, bedrooms, bathrooms, garage, basement, etc. In some embodiments, a phage disclosed herein is in the same manner as conventional cleaners. In some embodiments, the phage is applied in conjunction with (before, after, or simultaneously with) conventional cleaners provided that the conventional cleaner is formulated so as to preserve adequate bacteriophage biologic activity.

In some embodiments, a bacteriophage disclosed herein is added to a component of paper products, either during processing or after completion of processing of the paper products. Paper products to which a bacteriophage disclosed herein is added include, but are not limited to, paper towels, toilet paper, moist paper wipes.

### Food Safety

In some embodiments, a bacteriophage described herein is used in any food product or nutritional supplement, for preventing contamination. Examples for food or pharmaceuticals products are milk, yoghurt, curd, cheese, fermented milks, milk based fermented products, ice-creams, fermented cereal based products, milk based powders, infant formulae or tablets, liquid suspensions, dried oral supplement, wet oral supplement, or dry-tube-feeding.

The broad concept of bacteriophage sanitation is applicable to other agricultural applications and organisms. Produce, including fruits and vegetables, dairy products, and other agricultural products. For example, freshly-cut produce frequently arrive at the processing plant contaminated with pathogenic bacteria. This has led to outbreaks of food-borne illness traceable to produce. In some embodiments, the application of bacteriophage preparations to agricultural produce substantially reduce or eliminate the possibility of food-borne illness through application of a single phage or phage mixture with specificity toward species of bacteria associated with food-borne illness. In some embodiments, bacteriophages are applied at various stages of production and processing to reduce bacterial contamination at that point or to protect against contamination at subsequent points.

In some embodiments, specific bacteriophages are applied to produce in restaurants, grocery stores, produce distribution centers. In some embodiments, bacteriophages disclosed herein are periodically or continuously applied to the fruit and vegetable contents of a salad bar. In some embodiments, the application of bacteriophages to a salad bar or to sanitize the exterior of a food item is a misting or spraying process or a washing process.

In some embodiments, a bacteriophage described herein is used in matrices or support media containing with packaging containing meat, produce, cut fruits and vegetables, and other foodstuffs. In some embodiments, polymers that are suitable for packaging are impregnated with a bacteriophage preparation.

In some embodiments, a bacteriophage described herein is used in farm houses and livestock feed. In some embodiments, on a farm raising livestock, the livestock is provided with bacteriophage in their drinking water, food, or both. In some embodiments, a bacteriophage described herein is sprayed onto the carcasses and used to disinfect the slaughter area.

The use of specific bacteriophages as biocontrol agents on produce provides many advantages. For example, bacteriophages are natural, non-toxic products that will not disturb the ecological balance of the natural microflora in the way the common chemical sanitizers do, but will specifically lyse the targeted food-borne pathogens. Because bacteriophages, unlike chemical sanitizers, are natural products that evolve along with their host bacteria, new phages that are active against recently emerged, resistant bacteria are rapidly identified when required, whereas identification of a new effective sanitizer is a much longer process, several years.

### Pharmaceutical Compositions

Disclosed herein, in certain embodiments, are pharmaceutical compositions comprising (a) the nucleic acid sequences as disclosed herein; and (b) a pharmaceutically acceptable excipient. Also disclosed herein, in certain embodiments, are pharmaceutical compositions comprising (a) the bacteriophages as disclosed herein; and (b) a pharmaceutically acceptable excipient. Further disclosed herein, in certain embodiments, are pharmaceutical compositions comprising (a) the compositions as disclosed herein; and (b) a pharmaceutically acceptable excipient.

In some embodiments, the disclosure provides pharmaceutical compositions and methods of administering the same to treat bacterial, archaeal infections or to disinfect an area. In some embodiments, the pharmaceutical composition comprises any of the reagents discussed above in a pharmaceutically acceptable carrier. In some embodiments, a pharmaceutical composition or method disclosed herein treats urinary tract infections (UTI) and/or inflammatory diseases (e.g. inflammatory bowel disease (IBD)). In some embodiments, a pharmaceutical composition or method disclosed herein treats Crohn's disease. In some embodiments, a pharmaceutical composition or method disclosed herein treats ulcerative colitis.

In some embodiments, compositions disclosed herein comprise medicinal agents, pharmaceutical agents, carriers, adjuvants, dispersing agents, diluents, and the like.

In some embodiments, the bacteriophages disclosed herein are formulated for administration in a pharmaceutical carrier in accordance with suitable methods. In some embodiments, the manufacture of a pharmaceutical composition according to the disclosure, the bacteriophage is admixed with, inter alia, an acceptable carrier. In some embodiments, the carrier is a solid (including a powder) or a liquid, or both, and is preferably formulated as a unit-dose composition. In some embodiments, one or more bacteriophages are incorporated in the compositions disclosed herein, which are prepared by any suitable method of a pharmacy.

In some embodiment, a method of treating subject's in-vivo, comprising administering to a subject a pharmaceutical composition comprising a bacteriophage disclosed herein in a pharmaceutically acceptable carrier, wherein the pharmaceutical composition is administered in a therapeutically effective amount. In some embodiments, the administration of the bacteriophage to a human subject or an animal in need thereof are by any means known in the art.

In some embodiments, bacteriophages disclosed herein are for oral administration. In some embodiments, the bacteriophages are administered in solid dosage forms, such as capsules, tablets, and powders, or in liquid dosage forms, such as elixirs, syrups, and suspensions. In some embodiments, compositions and methods suitable for buccal (sub-lingual) administration include lozenges comprising the bacteriophages in a flavored base, usually sucrose and acacia or tragacanth; and pastilles comprising the bacteriophages in an inert base such as gelatin and glycerin or sucrose and acacia.

In some embodiments, methods and compositions of the present disclosure are suitable for parenteral administration comprising sterile aqueous and non-aqueous injection solutions of the bacteriophage. In some embodiments, these preparations are isotonic with the blood of the intended recipient. In some embodiments, these preparations comprise antioxidants, buffers, bacteriostals and solutes which render the composition isotonic with the blood of the intended recipient. In some embodiments, aqueous and non-aqueous sterile suspensions include suspending agents and thickening agents. In some embodiments, compositions disclosed herein are presented in unit\dose or multi-dose containers, for example sealed ampoules and vials, and are stored in a freeze-dried(lyophilized) condition requiring only the addition of the sterile liquid carrier, for example, saline or water for injection on immediately prior to use.

In some embodiment, methods and compositions suitable for rectal administration are presented as unit dose suppositories. In some embodiments, these are prepared by admixing the bacteriophage with one or more conventional solid carriers, for example, cocoa butter, and then shaping the resulting mixture. In some embodiments, methods and compositions suitable for topical application to the skin are in the form of an ointment, cream, lotion, paste, gel, spray, aerosol, or oil. In some embodiments, carriers which are used include petroleum jelly, lanoline, polyethylene glycols, alcohols, transdermal enhancers, and combinations of two or more thereof.

In some embodiments, methods and compositions suitable for transdermal administration are presented as discrete patches adapted to remain in intimate contact with the epidermis of the recipient for a prolonged period of time.

In some embodiments, methods and compositions suitable for nasal administration or otherwise administered to the lungs of a subject include any suitable means, e.g., administered by an aerosol suspension of respirable particles comprising the bacteriophage compositions, which the subject inhales. In some embodiments, the respirable particles are liquid or solid. As used herein, “aerosol” includes any gas-borne suspended phase, which is capable of being inhaled into the bronchioles or nasal passages. In some embodiments, aerosols of liquid particles are produced by any suitable means, such as with a pressure-driven aerosol nebulizer or an ultrasonic nebulizer. In some embodiments, aerosols of solid particles comprising the composition is produced with any solid particulate medicament aerosol generator, by techniques known in the pharmaceutical art.

In some embodiment, methods and compositions suitable for administering bacteriophages disclosed herein to a surface of an object or subject includes aqueous solutions. In some embodiments, such aqueous solutions are sprayed onto the surface of an object or subject. In some embodiment, the aqueous solutions are used to irrigate and clean a physical wound of a subject form foreign debris including bacteria.

In some embodiments, the bacteriophages disclosed herein are administered to the subject in a therapeutically effective amount. In some embodiments, at least one bacteriophage composition disclosed herein is formulated as a pharmaceutical formulation. In some embodiments, a pharmaceutical formulation comprises 1, 2, 3, 4, 5, 6, 7, 8, 9, 10, 11, 12, 13, 14, 15, 16, 17, 18, 19, 20 or more bacteriophage disclosed herein. In some instances, a pharmaceutical formulation comprises a bacteriophage described herein and at least one of: an excipient, a diluent, or a carrier.

In some embodiments, a pharmaceutical formulation comprises an excipient. Excipients are described in the Handbook of Pharmaceutical Excipients, American Pharmaceutical Association (1986) and includes but are not limited to solvents, dispersion media, diluents, or other liquid vehicles, dispersion or suspension aids, surface active agents, isotonic agents, thickening or emulsifying agents, preservatives, solid binders, and lubricants.

Non-limiting examples of suitable excipients include but is not limited to a buffering agent, a preservative, a stabilizer, a binder, a compaction agent, a lubricant, a chelator, a dispersion enhancer, a disintegration agent, a flavoring agent, a sweetener, a coloring agent.

In some embodiments, an excipient is a buffering agent. Non-limiting examples of suitable buffering agents include but is not limited to sodium citrate, magnesium carbonate, magnesium bicarbonate, calcium carbonate, and calcium bicarbonate. In some embodiments, a pharmaceutical formulation comprises any one or more buffering agent listed: sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts.

In some embodiments an excipient is a preservative. Non-limiting examples of suitable preservatives include but is not limited to antioxidants, such as alpha-tocopherol and ascorbate, and antimicrobials, such as parabens, chlorobutanol, and phenol. In some embodiments, antioxidants include but not limited to Ethylenediaminetetraacetic acid (EDTA), citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some embodiments, preservatives include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, protease inhibitor, reducing agent, alkylating agent, antimicrobial agent, oxidase inhibitor, or other inhibitor.

In some embodiments, a pharmaceutical formulation comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments, the binders that are used in a pharmaceutical formulation are selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some embodiments, a pharmaceutical formulation comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethylene glycol, sodium benzoate, sodium lauryl sulfate, magnesium lauryl sulfate, and light mineral oil. In some embodiments, lubricants that are in a pharmaceutical formulation are selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments, an excipient comprises a flavoring agent. In some embodiments, flavoring agents includes natural oils; extracts from plants, leaves, flowers, and fruits; and combinations thereof.

In some embodiments, an excipient comprises a sweetener. Non-limiting examples of suitable sweeteners include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia rebaudiana (Stevioside); chloro derivatives of sucrose such as sucralose; and sugar alcohols such as sorbitol, mannitol, sylitol, and the like.

In some instances, a pharmaceutical formulation comprises a coloring agent. Non-limiting examples of suitable color agents include food, drug and cosmetic colors (FD&C), drug and cosmetic colors (D&C), and external drug and cosmetic colors (Ext. D&C).

In some embodiments, the pharmaceutical formulation disclosed herein comprises a chelator. In some embodiments, a chelator includes ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA.

In some instances, a pharmaceutical formulation comprises a diluent. Non-limiting examples of diluents include water, glycerol, methanol, ethanol, and other similar biocompatible diluents. In some embodiments, a diluent is an aqueous acid such as acetic acid, citric acid, maleic acid, hydrochloric acid, phosphoric acid, nitric acid, sulfuric acid, or similar.

In some embodiments, a pharmaceutical formulation comprises a surfactant. In some embodiments, surfactants are be selected from, but not limited to, polyoxyethylene sorbitan fatty acid esters (polysorbates), sodium lauryl sulphate, sodium stearyl fumarate, polyoxyethylene alkyl ethers, sorbitan fatty acid esters, polyethylene glycols (PEG), polyoxyethylene castor oil derivatives, docusate sodium, quaternary ammonium compounds, amino acids such as L-leucine, sugar esters of fatty acids, glycerides of fatty acids or a combination thereof.

In some instances, a pharmaceutical formulation comprises an additional pharmaceutical agent. In some embodiments, an additional pharmaceutical agent is an antibiotic agent. In some embodiments, an antibiotic agent is of the group consisting of aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides or tetracycline.

In some embodiments, an antibiotic agent described herein is an aminoglycoside such as Amikacin, Gentamicin, Kanamycin, Neomycin, Netilmicin, Tobramycin or Paromomycin. In some embodiments, an antibiotic agent described herein is an Ansamycin such as Geldanamycin or Herbimycin.

In some embodiments, an antibiotic agent described herein is a carbacephem such as Loracarbef. In some embodiments, an antibiotic agent described herein is a carbapenem such as Ertapenem, Doripenem, Imipenem/Cilastatin or Meropenem.

In some embodiments, an antibiotic agent described herein is a cephalosporins (first generation) such as Cefadroxil, Cefazolin, Cefalexin, Cefalotin or Cefalothin, or alternatively a Cephalosporins (second generation) such as Cefaclor, Cefamandole, Cefoxitin, Cefprozil or Cefuroxime. In some embodiments, an antibiotic agent is a Cephalosporins (third generation) such as Cefixime, Cefdinir, Cefditoren, Cefoperazone, Cefotaxime, Cefpodoxime, Ceftibuten, Ceftizoxime and Ceftriaxone or a Cephalosporins (fourth generation) such as Cefepime or Ceftobiprole.

In some embodiments, an antibiotic agent described herein is a lincosamide such as Clindamycin and Azithromycin, or a macrolide such as Azithromycin, Clarithromycin, Dirithromycin, Erythromycin, Roxithromycin, Troleandomycin, Telithromycin and Spectinomycin.

In some embodiments, an antibiotic agent described herein is a monobactams such as Aztreonam, or a nitrofuran such as Furazolidone or Nitrofurantoin.

In some embodiments, an antibiotic agent described herein is a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin and Ticarcillin.

In some embodiments, an antibiotic agent described herein is a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, or Trimethoprim-Sulfamethoxazole (Co-trimoxazole) (TMP-SMX).

In some embodiments, an antibiotic agent described herein is a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin.

In some embodiments, an antibiotic agent described herein is a polypeptide such as Bacitracin, Colistin or Polymyxin B.

In some embodiments, an antibiotic agent described herein is a tetracycline such as Demeclocycline, Doxycycline, Minocycline or Oxytetracycline.

### Numbered Embodiments

Numbered embodiment 1 comprises a bacteriophage comprising a nucleic acid sequence encoding a Type I CRISPR-Cas system comprising: (a) CRISPR array; (b) a Cascade polypeptide; and (c) a Cas3 polypeptide. Numbered embodiment 2 comprises the bacteriophage of embodiment 1, wherein the CRISPR array comprises a spacer sequence and at least one repeat sequence. Numbered embodiment 3 comprises the bacteriophage of embodiment 2, wherein the at least one repeat sequence is operably linked to the spacer sequence at either its 5′ end or its 3′ end. Numbered embodiment 4 comprises the bacteriophage of any one of embodiments 1-3, wherein the spacer sequence is complementary to a target nucleotide sequence in a target bacterium. Numbered embodiment 5 comprises the bacteriophage of embodiments 1-4, wherein the target nucleotide sequence comprises a coding sequence. Numbered embodiment 6 comprises the bacteriophage of embodiments 1-5, wherein the target nucleotide sequence comprises a non-coding or intergenic sequence. Numbered embodiment 7 comprises the bacteriophage of embodiments 1-6, wherein the target nucleotide sequence comprises all or a part of a promoter sequence. Numbered embodiment 8 comprises the bacteriophage of embodiments 1-7, wherein the target nucleotide sequence comprises all or a part of a nucleotide sequence located on a coding strand of a transcribed region of an essential gene. Numbered embodiment 9 comprises the bacteriophage of embodiments 1-8 wherein the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK. Numbered embodiment 10 comprises the bacteriophage of any one of embodiments 1-9, wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system. Numbered embodiment 11 comprises the bacteriophage of any one of embodiments 1-10, wherein the Cascade complex comprises: (i) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (ii) a Cas6b polypeptide, a Cas8b polypeptide, a Cas7 polypeptide, and a Cas5 polypeptide (Type I-B CRISPR-Cas system); (iii) a Cas5d polypeptide, a Cas8c polypeptide, and a Cas7 polypeptide (Type I-C CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); (vi) a Csy1 polypeptide, a Csy2 polypeptide, a Csy3 polypeptide, and a Csy4 polypeptide (Type I-F CRISPR-Cas system). Numbered embodiment 12 comprises the bacteriophage of embodiments 1-11, wherein the Cas complex comprises a Cas5d polypeptide, a Cas8c polypeptide, and a Cas7 polypeptide (Type I-C CRISPR-Cas system). Numbered embodiment 13 comprises the bacteriophage of any one of embodiments 1-12, wherein the nucleic acid sequence further comprises a promoter sequence. Numbered embodiment 14 comprises the bacteriophage of any one of embodiments 1-13, wherein the target bacterium is killed solely by lytic activity of the bacteriophage. Numbered embodiment 15 comprises the bacteriophage of any one of embodiments 1-14, wherein the target bacterium is killed solely by activity of the CRISPR-Cas system. Numbered embodiment 16 comprises the bacteriophage of any one of embodiments 1-15, wherein the target bacterium is killed by lytic activity of the bacteriophage in combination with activity of the CRISPR-Cas system. Numbered embodiment 17 comprises the bacteriophage of any one of embodiments 1-16, wherein the target bacterium is killed by the activity of the CRISPR-Cas system, independently of the lytic activity of the bacteriophage. Numbered embodiment 18 comprises the bacteriophage of any one of embodiments 1-17, wherein the activity of the CRISPR-Cas system supplements or enhances the lytic activity of the bacteriophage. Numbered embodiment 19 comprises the bacteriophage of any one of embodiments 1-18, wherein the lytic activity of the bacteriophage and the activity of the CRISPR-Cas system are synergistic. Numbered embodiment 20 comprises the bacteriophage of any one of embodiments 1-19, wherein the lytic activity of the bacteriophage, the activity of the CRISPR-Cas system, or both, is modulated by a concentration of the bacteriophage Numbered embodiment 21 comprises the bacteriophage of any one of embodiments 1-20, wherein the bacteriophage infects multiple bacterial strains. Numbered embodiment 22 comprises the bacteriophage of any one of embodiments 1-21, wherein the target bacterium is an Acinetobacter species, an Actinomyces species, Burkholderia cepacia complex, a Campylobacter species, a Candida species, Clostridium difficile, Corynebacterium minutissium, Corynebacterium pseudodiphtheriae, Corynebacterium stratium, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter felis, Helicobacter pylori, Clostridium bolteae, and any combination thereof. Numbered embodiment 23 comprises the bacteriophage of any one of embodiments 1-22, wherein the bacteriophage is an obligate lytic bacteriophage. Numbered embodiment 24 comprises the bacteriophage of any one of embodiments 1-23, wherein the bacteriophage is a temperate bacteriophage that is rendered lytic. Numbered embodiment 25 comprises the bacteriophage of embodiments 1-24, wherein the temperate bacteriophage is rendered lytic by the removal, replacement, or inactivation of a lysogeny gene. Numbered embodiment 26 comprises the bacteriophage of any one of embodiments 1-25 wherein the bacteriophage is p1772, p2131, p2132, p2973, p4209, p1106, p1587, p1835, p2037, p2421, p2363, p004k, or PB1. Numbered embodiment 27 comprises the bacteriophage of any one of embodiments 1-26, wherein the nucleic acid sequence is inserted into a non-essential bacteriophage gene. Numbered embodiment 28 comprises a pharmaceutical composition comprising: (a) the bacteriophage of any one of embodiments 1-27; and (b) a pharmaceutically acceptable excipient. Numbered embodiment 29 comprises the pharmaceutical composition of embodiments 1-28, wherein the pharmaceutical composition is in the form of a tablet, a liquid, a syrup, an oral formulation, an intravenous formulation, an intranasal formulation, an ocular formulation, an otic formulation, a subcutaneous formulation, an inhalable respiratory formulation, a suppository, and any combination thereof. Numbered embodiment 30 comprises a method of killing a target bacterium comprising introducing into the target bacterium a bacteriophage comprising a nucleic acid sequence encoding a Type I CRISPR-Cas system comprising: (a) a CRISPR array; (b) a Cascade polypeptide; and (c) a Cas3 polypeptide. Numbered embodiment 31 comprises the method of embodiments 1-30, wherein the CRISPR array comprises a spacer sequence and at least one repeat sequence. Numbered embodiment 32 comprises the method of embodiments 1-31, wherein the at least one repeat sequence is operably linked to the spacer sequence at either its 5′ end or its 3′ end. Numbered embodiment 33 comprises the method of any one of embodiments 1-32 wherein the spacer sequence is complementary to a target nucleotide sequence in a target bacterium. Numbered embodiment 34 comprises the method of embodiment 1-33, wherein the target nucleotide sequence comprises a coding sequence. Numbered embodiment 35 comprises the method of embodiments 1-34, wherein the target nucleotide sequence comprises a non-coding or intergenic sequence. Numbered embodiment 36 comprises the method of embodiment 1-35, wherein the target nucleotide sequence comprises all or a part of a promoter sequence. Numbered embodiment 37 comprises the method of embodiments 1-36, wherein the target nucleotide sequence comprises all or a part of a nucleotide sequence located on a coding strand of a transcribed region of an essential gene. Numbered embodiment 38 comprises the method of embodiments 1-37, wherein the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK. Numbered embodiment 39 comprises the method of any one of embodiments 1-38, wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system. Numbered embodiment 40 comprises the method of any one of embodiments 1-39, wherein the Cascade complex comprises: (i) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (ii) a Cas6b polypeptide, a Cas8b polypeptide, a Cas7 polypeptide, and a Cas5 polypeptide (Type I-B CRISPR-Cas system); (iii) a Cas5d polypeptide, a Cas8c polypeptide, and a Cas7 polypeptide (Type I-C CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); (vi) a Csy1 polypeptide, a Csy2 polypeptide, a Csy3 polypeptide, and a Csy4 polypeptide (Type I-F CRISPR-Cas system). Numbered embodiment 41 comprises the method of embodiments 1-40, wherein the Cascade complex comprises a Cas5d polypeptide, a Cas8c polypeptide, and a Cas7 polypeptide (Type I-C CRISPR-Cas system). Numbered embodiment 42 comprises the method of any one of embodiments 1-41, wherein the nucleic acid sequence further comprises a promoter sequence. Numbered embodiment 43 comprises the method of any one of embodiments 1-42, wherein the target bacterium is killed solely by activity of the CRISPR-Cas system. Numbered embodiment 44 comprises the method of any one of embodiments 1-43, wherein the target bacterium is killed by lytic activity of the bacteriophage in combination with activity of the CRISPR-Cas system. Numbered embodiment 45 comprises the method of any one of embodiments 1-43, wherein the target bacterium is killed by the activity of the CRISPR-Cas system, independently of the lytic activity of the bacteriophage. Numbered embodiment 46 comprises the method of any one of embodiments 1-45, wherein the activity of the CRISPR-Cas system supplements or enhances the lytic activity of the bacteriophage. Numbered embodiment 47 comprises the method of any one of embodiments 1-46, wherein the lytic activity of the bacteriophage and the activity of the CRISPR-Cas system are synergistic. Numbered embodiment 48 comprises the method of any one of embodiments 1-47, wherein the lytic activity of the bacteriophage, the activity of the CRISPR-Cas system, or both, is modulated by a concentration of the bacteriophage Numbered embodiment 49 comprises the method of any one of embodiments 1-48, wherein the bacteriophage infects multiple bacterial strains. Numbered embodiment 50 comprises the method of any one of embodiments 1-49, wherein the target bacterium is an Acinetobacter species, an Actinomyces species, Burkholderia cepacia complex, a Campylobacter species, a Candida species, Clostridium difficile, Corynebacterium minutissium, Corynebacterium pseudodiphtherias, Corynebacterium stratium, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter felis, Helicobacter pylori, Clostridium bolteae, and any combination thereof. Numbered embodiment 51 comprises the method of any one of embodiments 1-50, wherein the bacteriophage is an obligate lytic bacteriophage. Numbered embodiment 52 comprises the method of any one of embodiments 1-51, wherein the bacteriophage is a temperate bacteriophage that is rendered lytic. Numbered embodiment 53 comprises the method of embodiments 1-52, wherein the temperate bacteriophage is rendered lytic by the removal, replacement, or inactivation of a lysogeny gene. Numbered embodiment 54 comprises the method of any one of embodiments 1-53, wherein the bacteriophage is p1772, p2131, p2132, p2973, p4209, p1106, p1587, p1835, p2037, p2421, p2363, p4209, p004k, or PB1. Numbered embodiment 55 comprises the method of any one of embodiments 1-54, wherein the nucleic acid sequence is inserted in pace of or adjacent to a non-essential bacteriophage gene. Numbered embodiment 56 comprises the method of any one of embodiments 1-55, wherein a mixed population of bacterial cells comprises the target bacterium. Numbered embodiment 57 comprises a method of treating a disease in an individual in need thereof, the method comprising administering to the individual a bacteriophage comprising a nucleic acid sequence encoding a Type I CRISPR-Cas system comprising: (a) a CRISPR array; (b) a Cascade polypeptide; and (c) a Cas3 polypeptide. Numbered embodiment 58 comprises the method of embodiments 1-57, wherein the CRISPR array comprises a spacer sequence and at least one repeat sequence. Numbered embodiment 59 comprises the method of embodiments 1-58, wherein the at least one repeat sequence is operably linked to the spacer sequence at either its 5′ end or its 3′ end. Numbered embodiment 60 comprises the method of any one of embodiments 1-59, wherein the spacer sequence is complementary to a target nucleotide sequence in a target bacterium. Numbered embodiment 61 comprises the method of embodiments 1-60, wherein the target nucleotide sequence comprises a coding sequence. Numbered embodiment 62 comprises the method of embodiments 1-61, wherein the target nucleotide sequence comprises a non-coding or intergenic sequence. Numbered embodiment 63 comprises the method of embodiments 1-62, wherein the target nucleotide sequence comprises all or a part of a promoter sequence. Numbered embodiment 64 comprises the method of embodiments 1-63, wherein the target nucleotide sequence comprises all or a part of a nucleotide sequence located on a coding strand of a transcribed region of an essential gene. Numbered embodiment 65 comprises the method of embodiments 1-64, wherein the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK. Numbered embodiment 66 comprises the method of any one of embodiments 1-65, wherein the Cascade complex comprises Cascade polypeptides of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system. Numbered embodiment 67 comprises the method of any one of embodiments 1-66, wherein the Cascade complex comprises: (i) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (ii) a Cas6b polypeptide, a Cas8b polypeptide, a Cas7 polypeptide, and a Cas5 polypeptide (Type I-B CRISPR-Cas system); (iii) a Cas5d polypeptide, a Cas8c polypeptide, and a Cas7 polypeptide (Type I-C CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); (vi) a Csy1 polypeptide, a Csy2 polypeptide, a Csy3 polypeptide, and a Csy4 polypeptide (Type I-F CRISPR-Cas system). Numbered embodiment 68 comprises the method of embodiments 1-67, wherein the CASCADE complex comprises a Cas5d polypeptide, a Cas8c polypeptide, and a Cas7 polypeptide (Type I-C CRISPR-Cas system). Numbered embodiment 69 comprises the method of any one of embodiments 1-68, wherein the nucleic acid sequence further comprises a promoter sequence. Numbered embodiment 70 comprises the method of any one of embodiments 1-69, wherein the target bacterium is killed solely by activity of the CRISPR-Cas system. Numbered embodiment 71 comprises the method of any one of embodiments 1-70, wherein the target bacterium is killed by lytic activity of the bacteriophage in combination with activity of the CRISPR-Cas system. Numbered embodiment 72 comprises the method of any one of embodiments 1-71, wherein the target bacterium is killed by the activity of the CRISPR-Cas system, independently of the lytic activity of the bacteriophage. Numbered embodiment 73 comprises the method of any one of embodiments 1-72, wherein the activity of the CRISPR-Cas system supplements or enhances the lytic activity of the bacteriophage. Numbered embodiment 74 comprises the method of any one of embodiments 1-73, wherein the lytic activity of the bacteriophage and the activity of the CRISPR-Cas system are synergistic. Numbered embodiment 75 comprises the method of any one of embodiments 1-74, wherein the lytic activity of the bacteriophage, the activity of the CRISPR-Cas system, or both, is modulated by a concentration of the bacteriophage Numbered embodiment 76 comprises the method of any one of embodiments 1-75, wherein the bacteriophage infects multiple bacterial strains. Numbered embodiment 77 comprises the method of any one of embodiments 1-76, wherein the bacteriophage is an obligate lytic bacteriophage. Numbered embodiment 78 comprises the method of any one of embodiments 1-77, wherein the bacteriophage is a temperate bacteriophage that is rendered lytic. Numbered embodiment 79 comprises the method of embodiments 1-78, wherein the temperate bacteriophage is rendered lytic by the removal, replacement, or inactivation of a lysogeny gene. Numbered embodiment 80 comprises the method of any one of embodiments 1-79, wherein the bacteriophage is p1772, p2131, p2132, p2973, p4209, p1106, p1587, p1835, p2037, p2421, p2363, p4209, p004k, or PB1. Numbered embodiment 81 comprises the method of any one of embodiments 1-80, wherein the nucleic acid sequence is inserted in pace of or adjacent to a non-essential bacteriophage gene. Numbered embodiment 82 comprises the method of any one of embodiments 1-81, wherein the disease is a bacterial infection. Numbered embodiment 83 comprises the method of any one of embodiments 1-82, wherein the target bacterium causing the disease is a drug resistant bacterium that is resistant to at least one antibiotic. Numbered embodiment 84 comprises the method of embodiments 1-83, wherein the drug resistant bacterium is resistant to at least one antibiotic. Numbered embodiment 85 comprises the method of any one of embodiments 1-84, wherein the target bacterium causing the disease is a multidrug resistant bacterium. Numbered embodiment 86 comprises the method of embodiments 1-85, wherein the multi-drug resistant bacterium is resistant to at least one antibiotic. Numbered embodiment 87 comprises the method of any one of embodiments 1-86, wherein the antibiotic comprises a cephalosporin, a fluoroquinolone, a carbapenem, a colistin, an aminoglycoside, vancomycin, streptomycin, or methicillin. Numbered embodiment 88 comprises the method of any one of embodiments 1-87, wherein the target bacterium causing the bacterial infection is an Acinetobacter species, an Actinomyces species, Burkholderia cepacia complex, a Campylobacter species, a Candida species, Clostridium difficile, Corynebacterium minutissium, Corynebacterium pseudodiphtheriae, Corynebacterium stratium, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter felis, Helicobacter pylori, Clostridium bolteae, and any combination thereof. Numbered embodiment 89 comprises the method of embodiments 1-88, wherein the target bacterium causing the disease is Pseudomonas. Numbered embodiment 90 comprises the method of embodiments 1-89, wherein the target bacterium causing the disease is P. aeruginosa. Numbered embodiment 91 comprises the method of any one of embodiments 1-90, wherein the administering is intra-arterial, intravenous, intraurethral, intramuscular, oral, subcutaneous, inhalation, or any combination thereof. Numbered embodiment 92 comprises the method of any one of embodiments 1-91, wherein the individual is a mammal. Numbered embodiment 93 comprises the bacteriophage of embodiments 1-92, the bacteriophage comprising a nucleic acid sequence encoding a Type I CRISPR-Cas system comprising: (a) a CRISPR array; (b) a Cascade polypeptide comprising Cas5, Cas8c and Cas7; and (c) a Cas3 polypeptide. Numbered embodiment 94 comprises the bacteriophage of embodiments 1-93, wherein the CRISPR array comprises a spacer sequence and at least one repeat sequence. Numbered embodiment 95 comprises the bacteriophage of embodiments 1-94, wherein the at least one repeat sequence is operably linked to the spacer sequence at either its 5′ end or its 3′ end. Numbered embodiment 96 comprises the bacteriophage of any one of embodiments 1-95, wherein the spacer sequence is complementary to a target nucleotide sequence in a target bacterium. Numbered embodiment 97 comprises the bacteriophage of embodiments 1-96, wherein the target nucleotide sequence comprises a coding sequence. Numbered embodiment 98 comprises the bacteriophage of embodiments 1-97, wherein the target nucleotide sequence comprises a non-coding or intergenic sequence. Numbered embodiment 99 comprises the bacteriophage of embodiments 1-98, wherein the target nucleotide sequence comprises all or a part of a promoter sequence. Numbered embodiment 100 comprises the bacteriophage of embodiments 1-99, wherein the target nucleotide sequence comprises all or a part of a nucleotide sequence located on a coding strand of a transcribed region of an essential gene. Numbered embodiment 101 comprises the bacteriophage of embodiments 1-100 wherein the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK. Numbered embodiment 102 comprises the bacteriophage of any one of embodiments 1-101, wherein the nucleic acid sequence further comprises a promoter sequence. Numbered embodiment 103 comprises the bacteriophage of any one of embodiments 1-102, wherein the target bacterium is killed solely by lytic activity of the bacteriophage. Numbered embodiment 104 comprises the bacteriophage of any one of embodiments 1-103, wherein the target bacterium is killed solely by activity of the CRISPR-Cas system. Numbered embodiment 105 comprises the bacteriophage of any one of embodiments 1-104, wherein the target bacterium is killed by lytic activity of the bacteriophage in combination with activity of the CRISPR-Cas system. Numbered embodiment 106 comprises the bacteriophage of any one of embodiments 1-105, wherein the target bacterium is killed by the activity of the CRISPR-Cas system, independently of the lytic activity of the bacteriophage. Numbered embodiment 107 comprises the bacteriophage of any one of embodiments 1-106, wherein the activity of the CRISPR-Cas system supplements or enhances the lytic activity of the bacteriophage. Numbered embodiment 108 comprises the bacteriophage of any one of embodiments 1-107, wherein the lytic activity of the bacteriophage and the activity of the CRISPR-Cas system are synergistic. Numbered embodiment 109 comprises the bacteriophage of any one of embodiments 1-108, wherein the lytic activity of the bacteriophage, the activity of the CRISPR-Cas system, or both, is modulated by a concentration of the bacteriophage Numbered embodiment 110 comprises the bacteriophage of any one of embodiments 1-109, wherein the bacteriophage infects multiple bacterial strains. Numbered embodiment 111 comprises the bacteriophage of any one of embodiments 1-110, wherein the target bacterium is an Acinetobacter species, an Actinomyces species, Burkholderia cepacia complex, a Campylobacter species, a Candida species, Clostridium difficile, Corynebacterium minutissium, Corynebacterium pseudodiphtherias, Corynebacterium stratium, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter felis, Helicobacter pylori, Clostridium bolteae, and any combination thereof. Numbered embodiment 112 comprises the bacteriophage of any one of embodiments 1-111, wherein the bacteriophage is an obligate lytic bacteriophage. Numbered embodiment 113 comprises the bacteriophage of any one of embodiments 1-112, wherein the bacteriophage is a temperate bacteriophage that is rendered lytic. Numbered embodiment 114 comprises the bacteriophage of embodiments 1-113, wherein the temperate bacteriophage is rendered lytic by the removal, replacement, or inactivation of a lysogeny gene. Numbered embodiment 115 comprises the bacteriophage of any one of embodiments 1-114, wherein the bacteriophage is p1772, p2131, p2132, p2973, p4209, p1106, p1587, p1835, p2037, p2421, p2363, p4209, p004k, or PB1. Numbered embodiment 116 comprises the bacteriophage of any one of embodiments 1-115, wherein the nucleic acid sequence is inserted into a non-essential bacteriophage gene. Numbered embodiment 117 comprises a pharmaceutical composition comprising: (a) the bacteriophage of any one of embodiments 1-116; and (b) a pharmaceutically acceptable excipient. Numbered embodiment 118 comprises the pharmaceutical composition of embodiment 117, wherein the pharmaceutical composition is in the form of a tablet, a liquid, a syrup, an oral formulation, an intravenous formulation, an intranasal formulation, an ocular formulation, an otic formulation, a subcutaneous formulation, an inhalable respiratory formulation, a suppository, and any combination thereof. Numbered embodiment 119 comprises a method of sanitizing a surface in need thereof, the method comprising administering to the surface a bacteriophage comprising a nucleic acid sequence encoding a Type I CRISPR-Cas system comprising: (d) a CRISPR array; (e) a Cascade polypeptide; and (f) a Cas3 polypeptide, wherein the bacteriophage comprises the bacteriophage of any one of embodiments 1-118. Numbered embodiment 120 comprises the method of embodiment 119, wherein the surface is a hospital surface, a vehicle surface, an equipment surface, or an industrial surface. Numbered embodiment 121 comprises a method of preventing contamination in a food product or a nutritional supplement, the method comprising administering to the a food product or the nutritional supplement a bacteriophage comprising a nucleic acid sequence encoding a Type I CRISPR-Cas system comprising: (g) a CRISPR array; (h) a Cascade polypeptide; and (i) a Cas3 polypeptide, wherein the bacteriophage comprises the bacteriophage of embodiments 1-120. Numbered embodiment 122 comprises the method of embodiment 121, wherein the food product or nutritional supplement comprises milk, yoghurt, curd, cheese, fermented milks, milk based fermented products, ice-creams, fermented cereal based products, milk based powders, infant formulae or tablets, liquid suspensions, dried oral supplement, wet oral supplement, or dry-tube-feeding.

### EXAMPLES

### Example 1: Engineered Phage Used in this Application

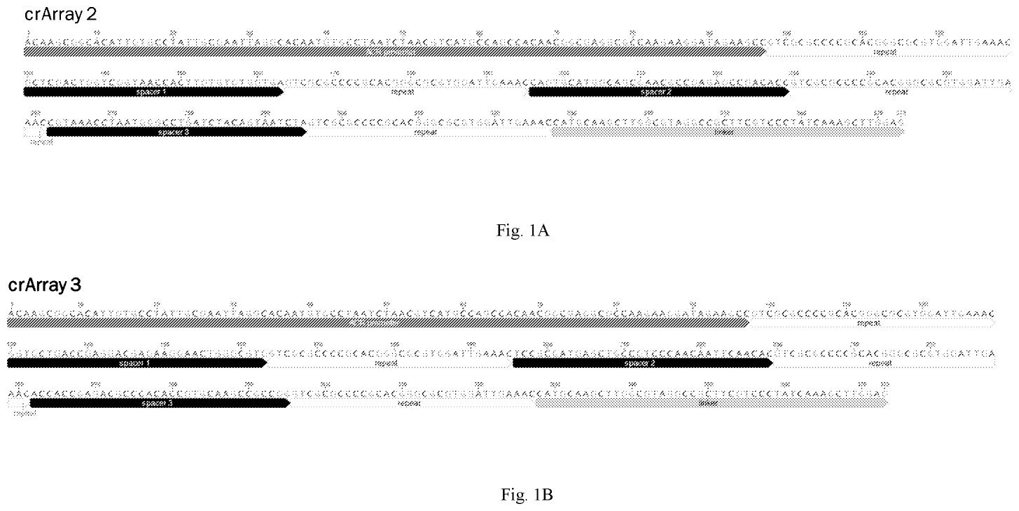

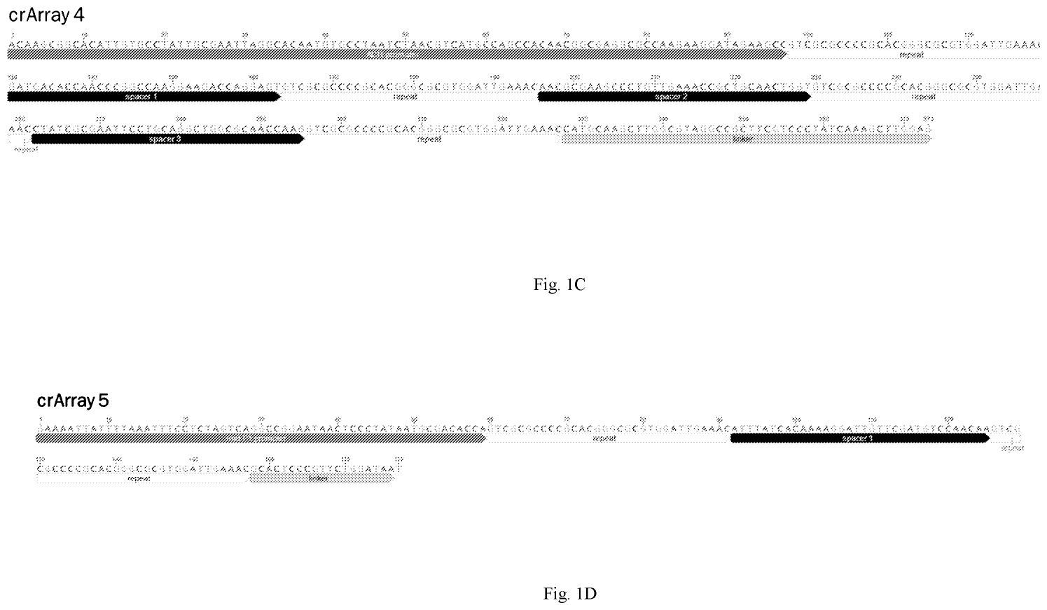

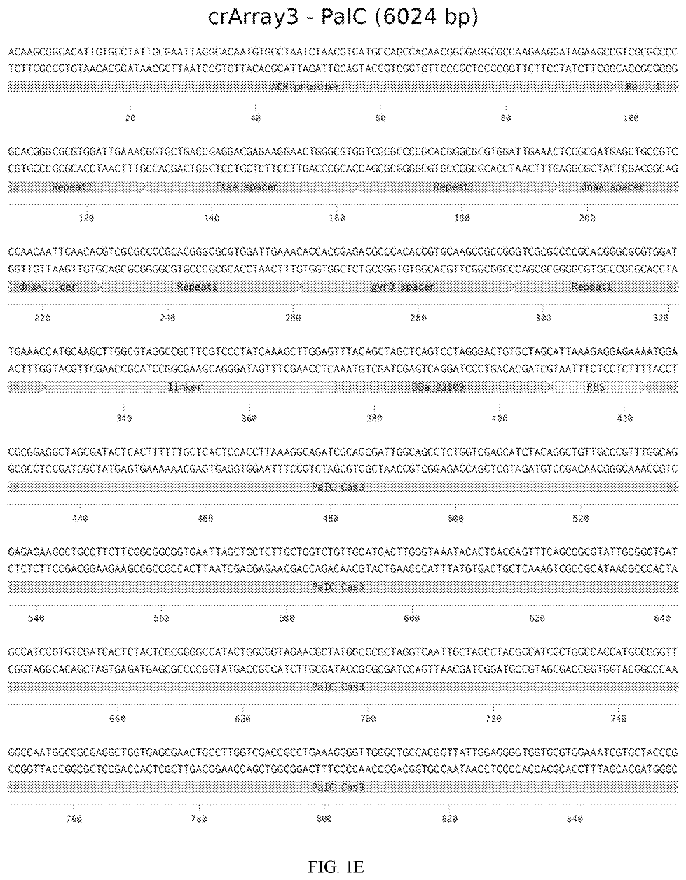

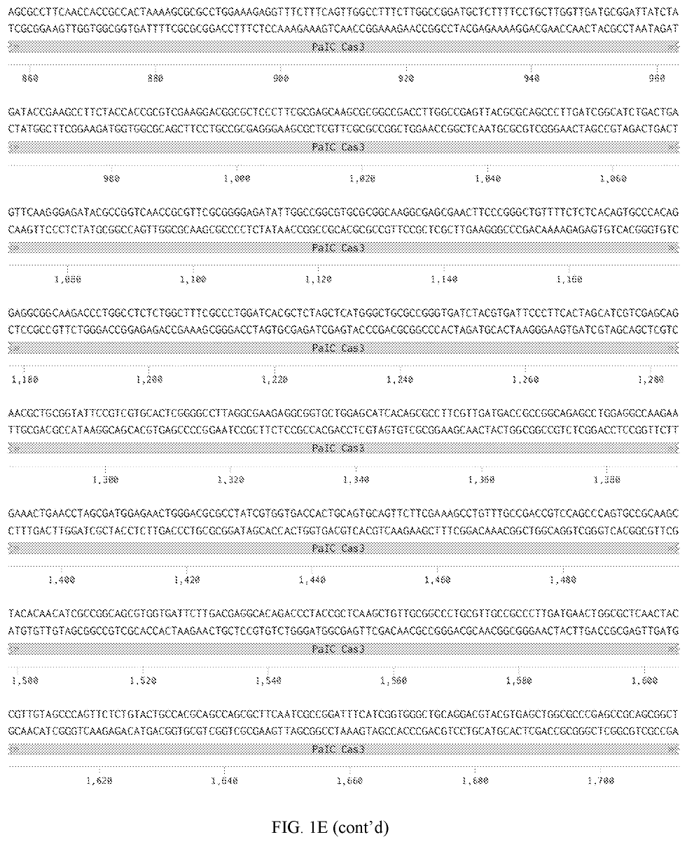

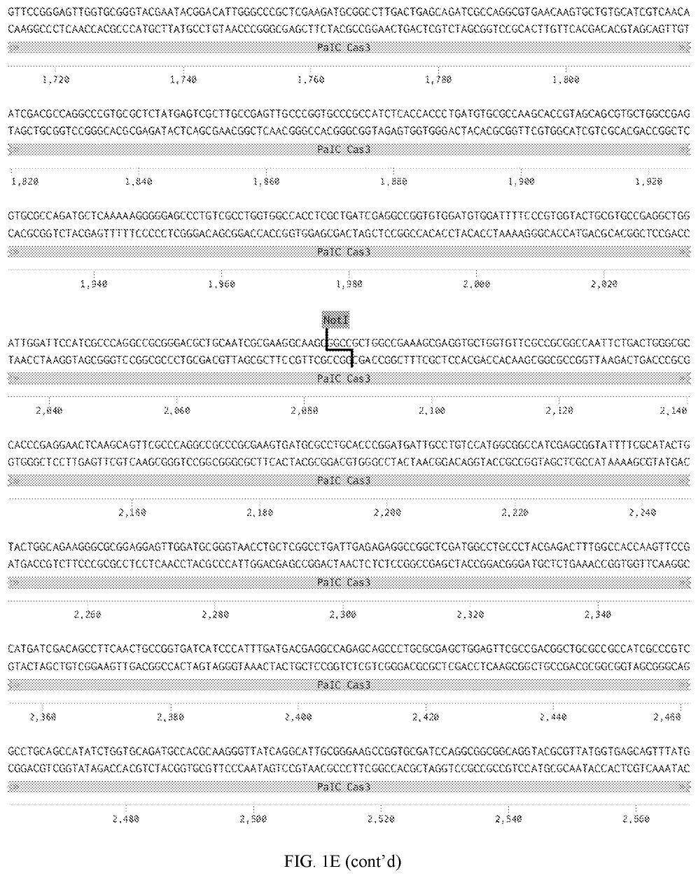

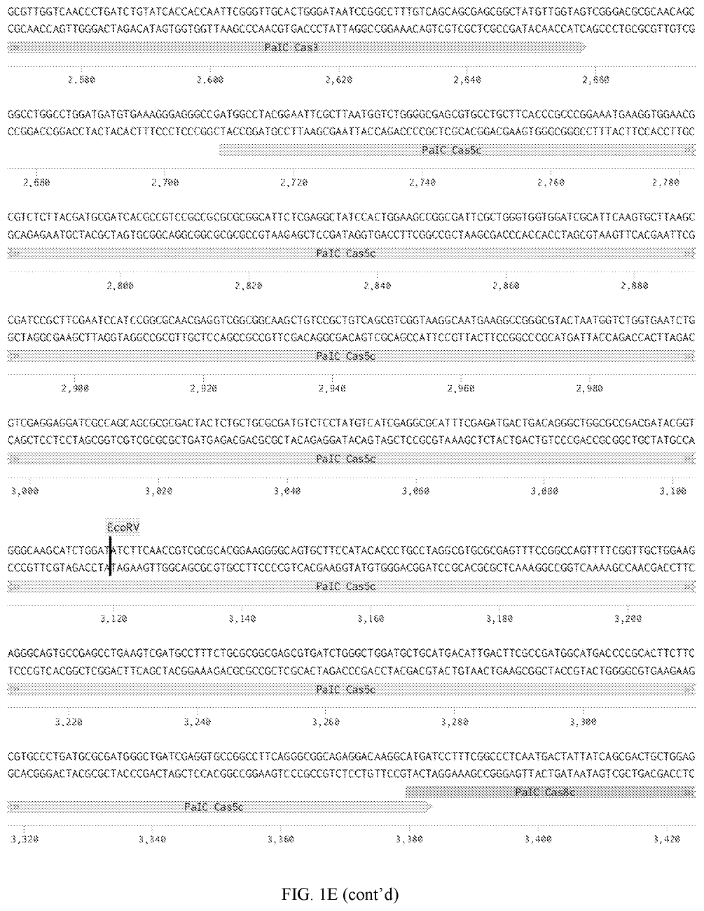

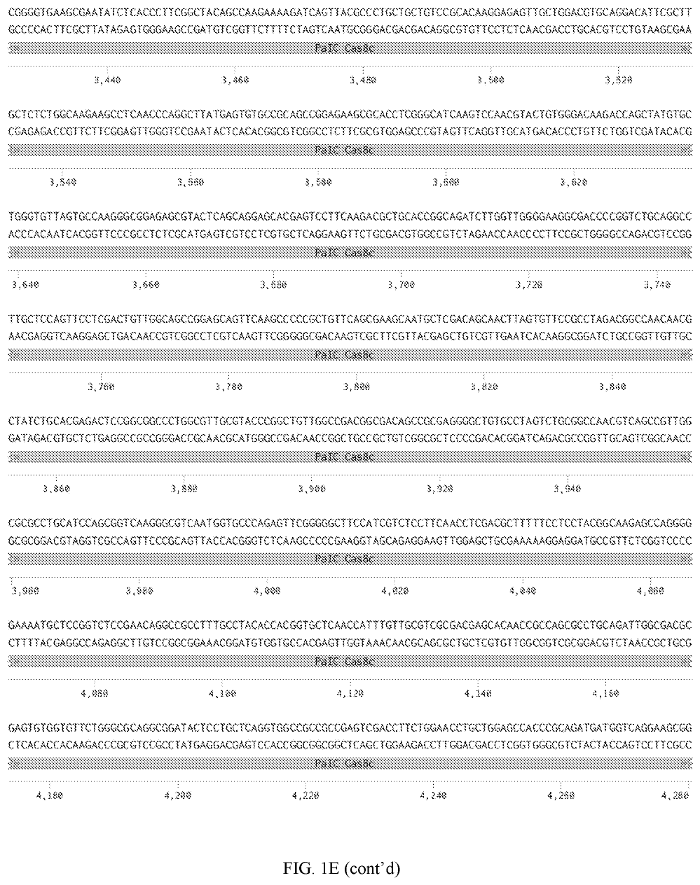

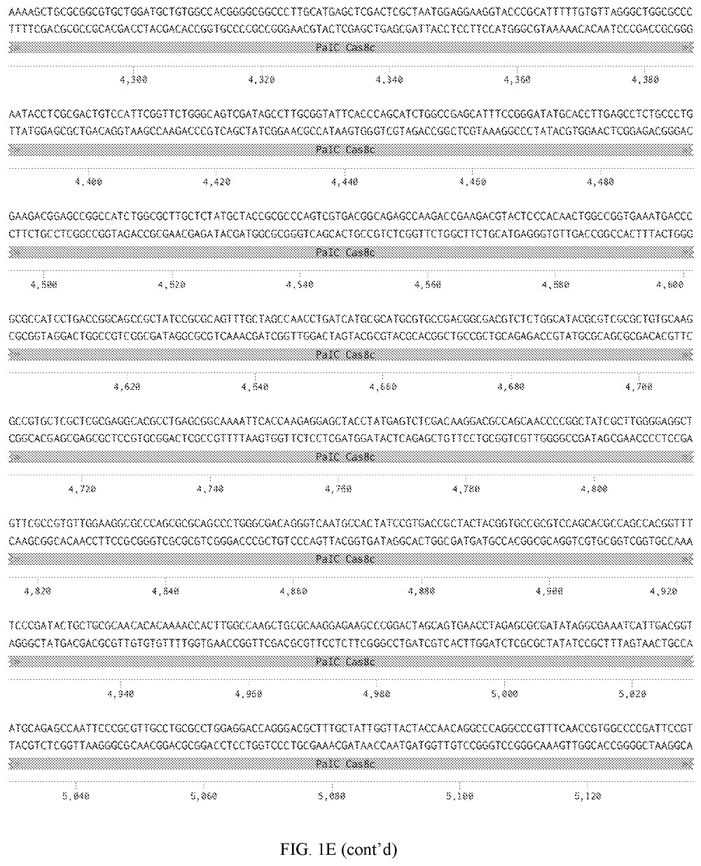

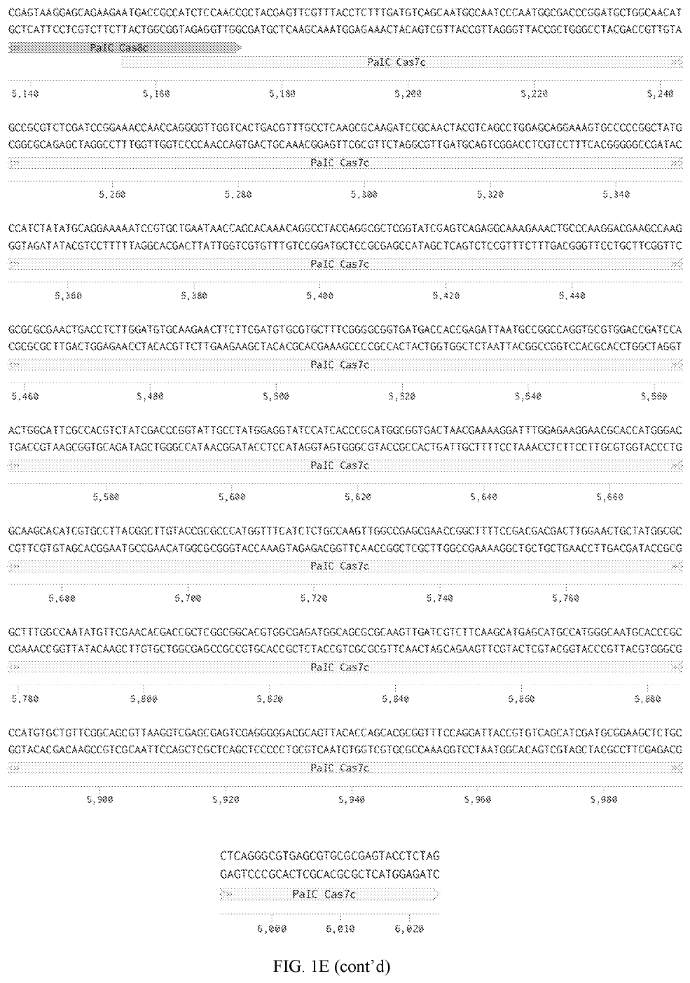

Bacteriophage were engineered to contain a crArray and Cas construct. Table 1A depicts the components of the phage used in the following application. Table 1B depicts the sequences of the promoters used to drive expression of both the crArray and the Cas promoter. Table 1C depicts the sequence of the spacer sequence in the crArray used to target specific sites. Further, FIGS. 1A-1D depict the sequence and alignment of crArray2-crArray5 used in the following examples. The full sequence of the combined crArray3 and Pseudomonas Type I C CRISPR insert is shown in FIG. 1E and Table 1D.

TABLE 1A
Components of phage

| | crArray | crArray | Cas | Cas |
|---|---|---|---|---|
| Phage name | promoter | ID | promoter | ID |
| p1772e004 | N/A | N/A | BBa_J23109 | PaIC |
| | | (no crArray) | | |
| p1772e005 | ACR | crArray 1 | BBa_J23109 | PaIC |
| p1772e006 | ACR | crArray 1 | N/A | N/A (no |
| | | | | Cas system) |
| p1772e008 | ACR | crArray 2 | BBa_J23109 | PaIC |
| p1772e016 | ACR | crArray 1 | endogenous | PaIC |
| p1772e017 | ACR | crArray 1 | BBa_J23106 | PaIC |
| p1772e018 | ACR | crArray 1 | P16 | PaIC |
| p1772e019 | ACR | crArray 1 | gp105 | PaIC |
| p1772e020 | ACR | crArray 1 | gp245 | PaIC |
| p1772e021 | ACR | crArray 1 | PAMP | PaIC |
| p1772e022 | ACR | crArray 1 | plpp | PaIC |
| p1772e023 | ACR | crArray 1 | ptat | PaIC |
| pArray3 | ACR | crArray 3 | BBa_J23109 | PaIC |
| pArray4 | ACR | crArray 4 | BBa_J23109 | PaIC |
| p2131e002 | ACR | crArray 1 | BBa_J23109 | PaIC |
| p2132e002 | ACR | crArray 1 | BBa_J23109 | PaIC |
| p2973e002 | ACR | crArray 1 | BBa_J23109 | PaIC |
| p4209e001 | N/A | N/A | N/A | PaIC |
| | | (no array) | | |
| p4209e002 | ACR | crArray 1 | BBa_J23109 | PaIC |
| PBIe002 | ACR | crArray 1 | BBa_J23109 | PaIC |
| p004ke007 | rrnB P1 | crArray 5 | BBa_J23109 | PaIC |

TABLE 1B
Promoter sequences

| SEQ | | | |
|---|---|---|---|
| ID | | | |
| NO | Promoter | Source | Sequence |
| 1 | ACR | phage | ACAAGCGGCA |
| | | genome | CATTGTGCCT |
| | | | ATTGCGAATT |
| | | | AGGCACAATG |
| | | | TGCCTAATCT |
| | | | AACGTCATGC |
| | | | CAGCCACAAC |
| | | | GGCGAGGCGC |
| | | | CAAGAAGGAT |
| | | | AGAAGCC |
| 2 | BBa_J23 | BioBrick | TTTACAGCTA |
| | 109 | | GCTCAGTCCT |
| | | | AGGGACTGTG |
| | | | CTAGC |
| 3 | endogenous | P. aeruginosa | GATTTTTTTC |
| | (promoter | genome | GGGTGAGGTT |
| | + RBS) | | GCGGGCTGTT |
| | | | CGGTAGGTTT |
| | | | ATAAACACTG |
| | | | CTATCCAAAG |
| | | | CTATGGACAC |
| | | | GCTCGGCTAC |
| | | | GAGAACAGTT |
| | | | GGCGTGATGG |
| | | | CCTCTAGCAA |
| | | | TTAGATTGTT |
| | | | ATGCGACATC |
| | | | CGCAGACTTG |
| | | | GCAGGGAGCG |
| | | | CACCT |
| 4 | BBa_J23106 | BioBrick | TTTACGGCTA |
| | | | GCTCAGTCCT |
| | | | AGGTATAGTG |
| | | | CTAGC |
| 5 | P16 | P. aeruginosa | ATCCGAGGGA |
| | (promoter | genome | TACGGGCCTT |
| | + RBS) | | GTCAGCACGG |
| | | | TGTTGCTAAT |
| | | | GAGAGCCTTT |
| | | | GCCCGGGCAA |
| | | | TAGTACGGGC |
| | | | AGTGTGTAGC |
| | | | GGATTGAAAG |
| | | | ACGCTGAATC |
| | | | ACTGACAGGC |
| | | | ATGAAGACTA |
| | | | TCGATAGAGT |
| | | | CTGATAGTGT |
| | | | CGCCGCCGCA |
| | | | CAGCGGATAG |
| | | | AGTCCACAGT |
| | | | CATTGAAGTG |
| | | | TTAATCCGCG |
| | | | ATCAAGCTC |
| 6 | gpl05 | phage | GACCTAGCTT |
| | (promoter | genome | TTATAGCGGG |
| | + RBS) | | TTTCGTGGTT |
| | | | TATAGCCCAT |
| | | | TGAAAAAAAT |
| | | | CTCACATCTA |
| | | | TATCACAGGT |
| | | | GTGCACTCGT |
| | | | TCCCGAAAGG |
| | | | TTCTGAGTCT |
| | | | ACTTGATCAA |
| | | | GTATTGAAAT |
| | | | ACCATCGTAA |
| | | | AGGAAAAAGA |
| | | | CATGTCTATT |
| | | | CGTGATAGCG |
| | | | AAAACAACAA |
| | | | CGGCCAACAG |
| | | | CAGCAGACCG |
| | | | CGCAAACTGC |
| | | | CGCCCCCGCC |
| | | | CCGCAA |
| 7 | gp245 | phage | TTCAATTTAA |
| | (promoter | genome | GTAGTAACGA |
| | + RBS) | | GGTCAGCCCG |
| | | | GAATCTTTGG |
| | | | GTATTCTTAA |
| | | | GGTATTTCTG |
| | | | ACTCAGTGTG |
| | | | GTTGGGACAG |
| | | | CTTCACTGTA |
| | | | CATTGCACTG |
| | | | GATTTGTTAA |
| | | | TTTCTTATAC |
| | | | CGGGGCACCA |
| | | | TGGGCAGCAA |
| | | | ATCGTGTTAC |
| | | | GAATTCCGTC |
| | | | TAACCAATAA |
| | | | GCGAGCTAAA |
| | | | TA |
| 8 | Pamp | plasmid | CGCGGAACCC |
| | | | CTATTTGTTT |
| | | | ATTTTTCTAA |
| | | | ATACATTCAA |
| | | | ATATGTATCC |
| | | | GCTCATGAGA |
| | | | CAATAACCCT |
| | | | GATAAATGCT |
| | | | TCAATAATAT |
| | | | TGAAAAAGGA |
| | | | AGAGT |
| 9 | PlPP | P. aeruginosa | CTTCAAGAAT |
| | (promoter | genome | TCGTATTGAC |
| | + RBS) | | CCCATAGACA |
| | | | GCTTCGTCGA |
| | | | CGCCCGTCCC |
| | | | GGCCCCCTTG |
| | | | GGCTTGCCGG |
| | | | ACGGCTTATG |
| | | | TCATGATGGC |
| | | | GCCACCCTCG |
| | | | CAGGTTCAAG |
| | | | GCCGGCTTTC |
| | | | TTCCTCTATG |
| | | | AACAAATCCC |
| | | | TTGCGCTGAC |
| | | | TACGTAATCA |
| | | | C |
| 10 | Ptat | P. aeruginosa | CTTCAAGAAT |
| | (promoter | genome | TCGGGGTATT |
| | + RBS) | | CCTGATCCTG |
| | | | CGCCGCTAGC |
| | | | GCCGCGCACG |
| | | | GCCACTAGGC |
| | | | CCGCGCCGAT |
| | | | AGCCAGTCGC |
| | | | GCTCCCGGCT |
| | | | GGCACACTAC |
| | | | TCCCATTTCC |
| | | | GCCGGAAACG |
| | | | CGCGCAACGT |
| | | | ACCGGCAACG |
| | | | AACGTGGAAA |
| | | | GACCATGAAA |
| | | | GACTGGCTGG |
| | | | ATGAGATTCA |
| | | | CTGGAACGCC |
| | | | GTGACCTACG |
| | | | TATGCAC |
| 11 | rrnB P1 | E. coli | GAAAATTATT |
| | | | TTAAATTTCC |
| | | | TCTAGTCAGG |
| | | | CCGGAATAAC |
| | | | TCCCTATAAT |
| | | | GCGACACCA |

TABLE 1C
Spacer sequences

| | | | |
|---|---|---|---|
| | | SEQ | |
| Array | Spacer | ID | |
| ID | Target | NO | Sequence |

Spacer 1

| | | | |
|---|---|---|---|
| crArray | Non- | 12 | GCTCGACTGGTCGGTAA |
| 2 | targeting | | CCACTTGTGTGTGGTG |
| | control | | A |
| crArray | ftsA | 13 | GGTGCTGACCGAGGACG |
| 3 | | | AGAAGGAACTGGGCG |
| | | | TG |
| crArra | glnS | 14 | GATGACACCAACCCGGC |
| y4 | | | CAAGGAAGACCAGGA |
| | | | GT |
| crArray | ppSa | 15 | ATTTATCACAAAAGGAT |
| 5 | | | TGTTCGATGTCCAACA |
| | | | A |

Spacer 2

| | | | |
|---|---|---|---|
| crArray | Non- | 16 | CAGTGCATGGCAGCGAA |
| 2 | targeting | | CGCCGAGAGCCGACA |
| | control | | CC |
| crArray | dnaA | 17 | TCCGCGATGAGCTGCCG |
| 3 | | | TCCCAACAATTCAACA |
| | | | C |
| crArray | dnaN | 18 | AACGCGAAGCCCTGTTG |
| 4 | | | AAACCGCTGCAACTGG |
| | | | T |

Spacer 3

| | | | |
|---|---|---|---|
| crArray | Non- | | CGTAAACCTAATGGGCC |
| 2 | targeting | | TGATCTACAGTAATCT |
| | control | 19 | A |
| crArray | gyrB | 20 | ACCACCGAGACGCCCAC |
| 3 | | | ACCGTGCAAGCCGCCG |
| | | | G |
| crArray | rpoB | 21 | CTATCGCGAATTCCTGC |
| 4 | | | AGGCTGGCGCAACCAA |
| | | | G |

TABLE ID
Sequence of crArray3-PAIC

Sequence of crArray3-PAIC (SEQ ID NO: 23)
ACAAGCGGCACATTGTGCCTATTGCGAATTAGGCAC
AATGTGCCTAATCTAACGTCATGCCAGCCACAACG
GCGAGGCGCCAAGAAGGATAGAAGCCGTCGCGCCC
CGCACGGGCGCGTGGATTGAAACGGTGCTGACCGA
GGACGAGAAGGAACTGGGCGTGGTCGCGCCCCGCA
CGGGCGCGTGGATTGAAACTCCGCGATGAGCTGCC
GTCCCAACAATTCAACACGTCGCGCCCCGCACGGG
CGCGTGGATTGAAACACCACCGAGACGCCCACACC
GTGCAAGCCGCCGGGTCGCGCCCCGCACGGGCGCG
TGGATTGAAACCATGCAAGCTTGGCGTAGGCCGCT
TCGTCCCTATCAAAGCTTGGAGTTTACAGCTAGCT
CAGTCCTAGGGACTGTGCTAGCATTAAAGAGGAGA
AAATGGACGCGGAGGCTAGCGATACTCACTTTTTT
GCTCACTCCACCTTAAAGGCAGATCGCAGCGATTG
GCAGCCTCTGGTCGAGCATCTACAGGCTGTTGCCC
GTTTGGCAGGAGAGAAGGCTGCCTTCTTCGGCGGC
GGTGAATTAGCTGCTCTTGCTGGTCTGTTGCATGA
CTTGGGTAAATACACTGACGAGTTTCAGCGGCGTA
TTGCGGGTGATGCCATCCGTGTCGATCACTCTACT
CGCGGGGCCATACTGGCGGTAGAACGCTATGGCGC
GCTAGGTCAATTGCTAGCCTACGGCATCGCTGGCC
ACCATGCCGGGTTGGCCAATGGCCGCGAGGCTGGT
GAGCGAACTGCCTTGGTCGACCGCCTGAAAGGGGT
TGGGCTGCCACGGTTATTGGAGGGGTGGTGCGTGG
AAATCGTGCTACCCGAGCGCCTTCAACCACCGCCA
CTAAAAGCGCGCCTGGAAAGAGGTTTCTTTCAGTT
GGCCTTTCTTGGCCGGATGCTCTTTTCCTGCTTGG
TTGATGCGGATTATCTAGATACCGAAGCCTTCTAC
CACCGCGTCGAAGGACGGCGCTCCCTTCGCGAGCA
AGCGCGGCCGACCTTGGCCGAGTTACGCGCAGCCC
TTGATCGGCATCTGACTGAGTTCAAGGGAGATACG
CCGGTCAACCGCGTTCGCGGGGAGATATTGGCCGG
CGTGCGCGGCAAGGCGAGCGAACTTCCCGGGCTGT
TTTCTCTCACAGTGCCCACAGGAGGCGGCAAGACC
CTGGCCTCTCTGGCTTTCGCCCTGGATCACGCTCT
AGCTCATGGGCTGCGCCGGGTGATCTACGTGATTC
CCTTCACTAGCATCGTCGAGCAGAACGCTGCGGTA
TTCCGTCGTGCACTCGGGGCCTTAGGCGAAGAGGC
GGTGCTGGAGCATCACAGCGCCTTCGTTGATGACC
GCCGGCAGAGCCTGGAGGCCAAGAAGAAACTGAAC
CTAGCGATGGAGAACTGGGACGCGCCTATCGTGGT
GACCACTGCAGTGCAGTTCTTCGAAAGCCTGTTTG
CCGACCGTCCAGCCCAGTGCCGCAAGCTACACAAC
ATCGCCGGCAGCGTGGTGATTCTTGACGAGGCACA
GACCCTACCGCTCAAGCTGTTGCGGCCCTGCGTTG
CCGCCCTTGATGAACTGGCGCTCAACTACCGTTGT
AGCCCAGTTCTCTGTACTGCCACGCAGCCAGCGCT
TCAATCGCCGGATTTCATCGGTGGGCTGCAGGACG
TACGTGAGCTGGCGCCCGAGCCGCAGCGGCTGTTC
CGGGAGTTGGTGCGGGTACGAATACGGACATTGGG
CCCGCTCGAAGATGCGGCCTTGACTGAGCAGATCG
CCAGGCGTGAACAAGTGCTGTGCATCGTCAACAAT
CGACGCCAGGCCCGTGCGCTCTATGAGTCGCTTGC
CGAGTTGCCCGGTGCCCGCCATCTCACCACCCTGA
TGTGCGCCAAGCACCGTAGCAGCGTGCTGGCCGAG
GTGCGCCAGATGCTCAAAAAGGGGGAGCCCTGTCG
CCTGGTGGCCACCTCGCTGATCGAGGCCGGTGTGG
ATGTGGATTTTCCCGTGGTACTGCGTGCCGAGGCT
GGATTGGATTCCATCGCCCAGGCCGCGGGACGCTG
CAATCGCGAAGGCAAGCGGCCGCTGGCCGAAAGCG
AGGTGCTGGTGTTCGCCGCGGCCAATTCTGACTGG
GCGCCACCCGAGGAACTCAAGCAGTTCGCCCAGGC
CGCCCGCGAAGTGATGCGCCTGCACCCGGATGATT
GCCTGTCCATGGCGGCCATCGAGCGGTATTTTCGC
ATACTGTACTGGCAGAAGGGCGCGGAGGAGTTGGA
TGCGGGTAACCTGCTCGGCCTGATTGAGAGAGGCC
GGCTCGATGGCCTGCCCTACGAGACTTTGGCCACC
AAGTTCCGCATGATCGACAGCCTTCAACTGCCGGT
GATCATCCCATTTGATGACGAGGCCAGAGCAGCCC
TGCGCGAGCTGGAGTTCGCCGACGGCTGCGCCGCC
ATCGCCCGTCGCCTGCAGCCATATCTGGTGCAGAT
GCCACGCAAGGGTTATCAGGCATTGCGGGAAGCCG
GTGCGATCCAGGCGGCGGCAGGTACGCGTTATGGT
GAGCAGTTTATGGCGTTGGTCAACCCTGATCTGTA
TCACCACCAATTCGGGTTGCACTGGGATAATCCGG
CCTTTGTCAGCAGCGAGCGGCTATGTTGGTAGTCG
GGACGCGCAACAGCGGCCTGGCCTGGATGATGTGA
AAGGGAGGGCCGATGGCCTACGGAATTCGCTTAAT
GGTCTGGGGCGAGCGTGCCTGCTTCACCCGCCCGG
AAATGAAGGTGGAACGCGTCTCTTACGATGCGATC
ACGCCGTCCGCCGCGCGCGGCATTCTCGAGGCTAT
CCACTGGAAGCCGGCGATTCGCTGGGTGGTGGATC
GCATTCAAGTGCTTAAGCCGATCCGCTTCGAATCC
ATCCGGCGCAACGAGGTCGGCGGCAAGCTGTCCGC
TGTCAGCGTCGGTAAGGCAATGAAGGCCGGGCGTA
CTAATGGTCTGGTGAATCTGGTCGAGGAGGATCGC
CAGCAGCGCGCGACTACTCTGCTGCGCGATGTCTC
CTATGTCATCGAGGCGCATTTCGAGATGACTGACA
GGGCTGGCGCCGACGATACGGTGGGCAAGCATCTG
GATATCTTCAACCGTCGCGCACGGAAGGGGCAGTG
CTTCCATACACCCTGCCTAGGCGTGCGCGAGTTTC
CGGCCAGTTTTCGGTTGCTGGAAGAGGGCAGTGCC
GAGCCTGAAGTCGATGCCTTTCTGCGCGGCGAGCG
TGATCTGGGCTGGATGCTGCATGACATTGACTTCG
CCGATGGCATGACCCCGCACTTCTTCCGTGCCCTG
ATGCGCGATGGGCTGATCGAGGTGCCGGCCTTCAG
GGCGGCAGAGGACAAGGCATGATCCTTTCGGCCCT
CAATGACTATTATCAGCGACTGCTGGAGCGGGGTG
AAGCGAATATCTCACCCTTCGGCTACAGCCAAGAA
AAGATCAGTTACGCCCTGCTGCTGTCCGCACAAGG
AGAGTTGCTGGACGTGCAGGACATTCGCTTGCTCT
CTGGCAAGAAGCCTCAACCCAGGCTTATGAGTGTG
CCGCAGCCGGAGAAGCGCACCTCGGGCATCAAGTC
CAACGTACTGTGGGACAAGACCAGCTATGTGCTGG
GTGTTAGTGCCAAGGGCGGAGAGCGTACTCAGCAG
GAGCACGAGTCCTTCAAGACGCTGCACCGGCAGAT
CTTGGTTGGGGAAGGCGACCCCGGTCTGCAGGCCT
TGCTCCAGTTCCTCGACTGTTGGCAGCCGGAGCAG
TTCAAGCCCCCGCTGTTCAGCGAAGCAATGCTCGA
CAGCAACTTAGTGTTCCGCCTAGACGGCCAACAAC
GCTATCTGCACGAGACTCCGGCGGCCCTGGCGTTG
CGTACCCGGCTGTTGGCCGACGGCGACAGCCGCGA
GGGGCTGTGCCTAGTCTGCGGCCAACGTCAGCCGT
TGGCGCGCCTGCATCCAGCGGTCAAGGGCGTCAAT
GGTGCCCAGAGTTCGGGGGCTTCCATCGTCTCCTT
CAACCTCGACGCTTTTTCCTCCTACGGCAAGAGCC
AGGGGGAAAATGCTCCGGTCTCCGAACAGGCCGCC
TTTGCCTACACCACGGTGCTCAACCATTTGTTGCG
TCGCGACGAGCACAACCGCCAGCGCCTGCAGATTG
GCGACGCGAGTGTGGTGTTCTGGGCGCAGGCGGAT
ACTCCTGCTCAGGTGGCCGCCGCCGAGTCGACCTT
CTGGAACCTGCTGGAGCCACCCGCAGATGATGGTC
AGGAAGCGGAAAAGCTGCGCGGCGTGCTGGATGCT
GTGGCCACGGGGCGGCCCTTGCATGAGCTCGACTC
GCTAATGGAGGAAGGTACCCGCATTTTTGTGTTAG
GGCTGGCGCCCAATACCTCGCGACTGTCCATTCGG
TTCTGGGCAGTCGATAGCCTTGCGGTATTCACCCA
GCATCTGGCCGAGCATTTCCGGGATATGCACCTTG
AGCCTCTGCCCTGGAAGACGGAGCCGGCCATCTGG
CGCTTGCTCTATGCTACCGCGCCCAGTCGTGACGG
CAGAGCCAAGACCGAAGACGTACTCCCACAACTGG
CCGGTGAAATGACCCGCGCCATCCTGACCGGCAGC
CGCTATCCGCGCAGTTTGCTAGCCAACCTGATCAT
GCGCATGCGTGCCGACGGCGACGTCTCTGGCATAC
GCGTCGCGCTGTGCAAGGCCGTGCTCGCTCGCGAG
GCACGCCTGAGCGGCAAAATTCACCAAGAGGAGCT
ACCTATGAGTCTCGACAAGGACGCCAGCAACCCCG
GCTATCGCTTGGGGAGGCTGTTCGCCGTGTTGGAA
GGCGCCCAGCGCGCAGCCCTGGGCGACAGGGTCAA
TGCCACTATCCGTGACCGCTACTACGGTGCCGCGT
CCAGCACGCCAGCCACGGTTTTCCCGATACTGCTG
CGCAACACACAAAACCACTTGGCCAAGCTGCGCAA
GGAGAAGCCCGGACTAGCAGTGAACCTAGAGCGCG
ATATAGGCGAAATCATTGACGGTATGCAGAGCCAA
TTCCCGCGTTGCCTGCGCCTGGAGGACCAGGGACG
CTTTGCTATTGGTTACTACCAACAGGCCCAGGCCC
GTTTCAACCGTGGCCCCGATTCCGTCGAGTAAGGA
GCAGAAGAATGACCGCCATCTCCAACCGCTACGAG
TTCGTTTACCTCTTTGATGTCAGCAATGGCAATCC
CAATGGCGACCCGGATGCTGGCAACATGCCGCGTC
TCGATCCGGAAACCAACCAGGGGTTGGTCACTGAC
GTTTGCCTCAAGCGCAAGATCCGCAACTACGTCAG
CCTGGAGCAGGAAAGTGCCCCCGGCTATGCCATCT
ATATGCAGGAAAAATCCGTGCTGAATAACCAGCAC
AAACAGGCCTACGAGGCGCTCGGTATCGAGTCAGA
GGCAAAGAAACTGCCCAAGGACGAAGCCAAGGCGC
GCGAACTGACCTCTTGGATGTGCAAGAACTTCTTC
GATGTGCGTGCTTTCGGGGCGGTGATGACCACCGA
GATTAATGCCGGCCAGGTGCGTGGACCGATCCAAC
TGGCATTCGCCACGTCTATCGACCCGGTATTGCCT
ATGGAGGTATCCATCACCCGCATGGCGGTGACTAA
CGAAAAGGATTTGGAGAAGGAACGCACCATGGGAC
GCAAGCACATCGTGCCTTACGGCTTGTACCGCGCC
CATGGTTTCATCTCTGCCAAGTTGGCCGAGCGAAC
CGGCTTTTCCGACGACGACTTGGAACTGCTATGGC
GCGCTTTGGCCAATATGTTCGAACACGACCGCTCG
GCGGCACGTGGCGAGATGGCAGCGCGCAAGTTGAT
CGTCTTCAAGCATGAGCATGCCATGGGCAATGCAC
CCGCCCATGTGCTGTTCGGCAGCGTTAAGGTCGAG
CGAGTCGAGGGGGACGCAGTTACACCAGCACGCGG
TTTCCAGGATTACCGTGTCAGCATCGATGCGGAAG
CTCTGCCTCAGGGCGTGAGCGTGCGCGAGTACCTC
TAG

### Example 2: Exogenous Cas Operon and crRNA Spacers Killed Bacteria

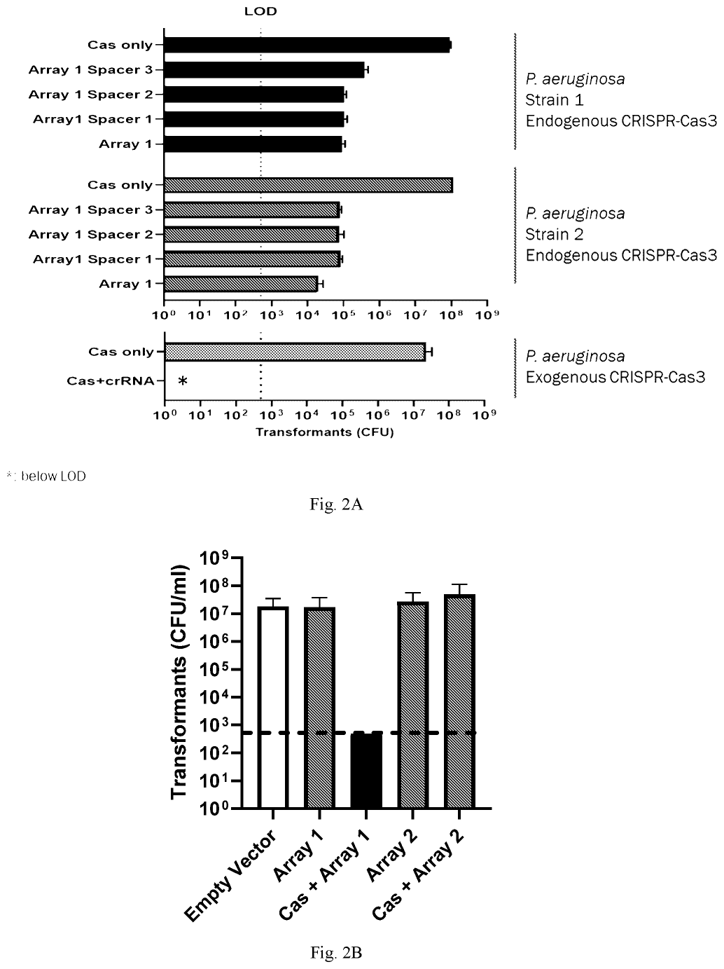

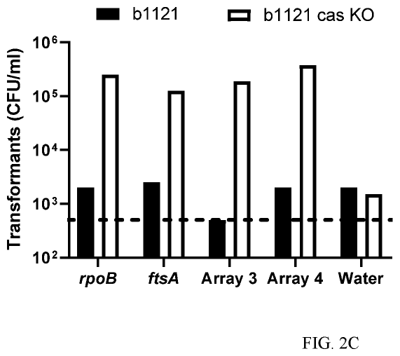

P. aeruginosa strains with a functional Type I-C Cas operon were transformed with Cas-only or crRNA-containing plasmids. In the presence of an endogenous Type I-C Cas system, the expression of a crRNA causes the bacteria to self-target and degrade its own DNA. The number of transformants was determined by counting the number of colonies that grow on an agar plate with antibiotic selection specific to the plasmid. The only bacteria that can form colonies are those that both acquire the plasmid and survive self-targeting. These data show that exogenous Cas expression improved self-targeting over the endogenous system and functions when the endogenous system is not present, as seen in FIG. 2A. The top two panels showed the results of transforming a Cas-only plasmid, plasmids containing single targeting spacers, or a plasmid containing a 3-spacer targeting array into strains containing an endogenous Type I-C Cas system. In this experimental set-up, the individual spacers or the array acted with the endogenous Cas system and were sufficient to kill most transformants. The bottom panel shows that the addition of an exogenous Type I-C Cas operon to the crRNA plasmid further enhanced kill upon transformation, with the level of bacteria present being below the level of detection.

The plasmids were transformed into a P. aeruginosa strain that did not contain a functional Type I-C Cas operon. The transformed plasmids expressed either a spacer array alone or the spacer array and the Type I-C Cas operon. FIG. 2B shows the number of bacterial transformants obtained per mL of transformation into a Cas operon null mutant of P. aeruginosa strain b1121. Array 1 targets the bacteria while array 2 is a non-targeting control. The different plasmids were normalized by molarity to the empty vector control plasmid. When cells were transformed with both Cas and targeting array 1, there was a decrease in the number of transformants detected. Cells transfected with only targeting array 1, or with Cas and the non-targeting array 2 did not show a decrease in number of transformants when compared to the number of transformants received with an empty vector.

Plasmids containing individual spacers or unique arrays were transformed into P. aeruginosa strain b1121, which has an endogenous Type I-C Cas system, or a Cas operon null mutant of the same strain. Cell death was observed in b1121 transfected cells, but not in the Cas operon null mutant. These data, as depicted in FIG. 2C indicated that individual spacers targeting rpoB and ftsA, as well as Array 3 and Array 4, were able to work with the endogenous Type I-C Cas system to kill the cells.

### Example 3: Stability of Phage Engineered with CRISPR-Cas Full Construct

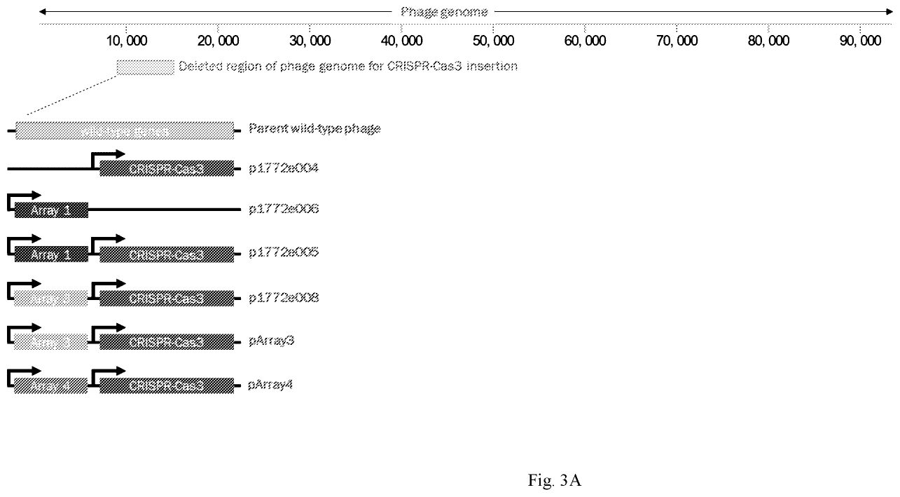

FIG. 3A depicts a schematic representation of the genome of wild type phage p1772 and its engineered variants. The bar below the genome axis indicates the region of the genome that was removed and replaced. The schematics below the phage genome illustrate the DNA that was used to replace WT phage genes in the deleted region. CRISPR arrays crArray 1, crArray 3, and crArray 4 target the bacteria and are expected to kill bacteria in the presence of an active Type I-C Cas system. crArray 2 is made from non-targeting spacers, but is structurally the same as the three targeting arrays, and serves as a control to demonstrate Type I Cas specific self-targeting activity.

Phage carrying the CRISPR-Cas3 construct were serially passaged to assess the stability of the repeats contained in the phage genome. p1772e005 was serially amplified on P. aeruginosa strain b1126 (a Type I-F strain). Amplifications one through eight were performed as one step amplifications where 50 uL of a bacterial overnight culture was added to 5 mL of LB in a 15 mL falcon tube followed by the immediate addition of 1 μL of the previous lysate. The mixtures were grown for 10-16 hours at 37° C. in a shaking incubator. Following incubation, phage-bacterial mixtures were centrifuged for 10 min at 5,000 rcf and the supernatants were filtered through 0.45 μm syringe filters and stored at 4° C. For amplification nine, serial ten-fold dilutions of amplification eight were spotted onto soft agar overlays of strain b1121 or b1126. A single plaque from each plate was picked with a pipette into 200 μL of PBS to obtain amplification nine. Ten microliters of amplification nine, were added to 50 μL of b1121 or b1126 overnight and 5 mL of LB, then grown for ˜16 hours followed by centrifugation and filtration. For sanger sequencing, phage DNA was amplified by PCR from lysates using primers flanking the engineering site. Sanger and NGS sequencing confirmed stability and integrity of the CRISPR-Cas3 construct when loaded onto the phage genome (shown in FIG. 3B).

### Example 4. Bacteriophage Morphology with CRISPR Constructs

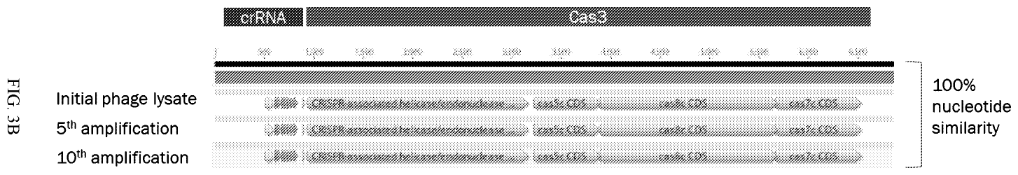

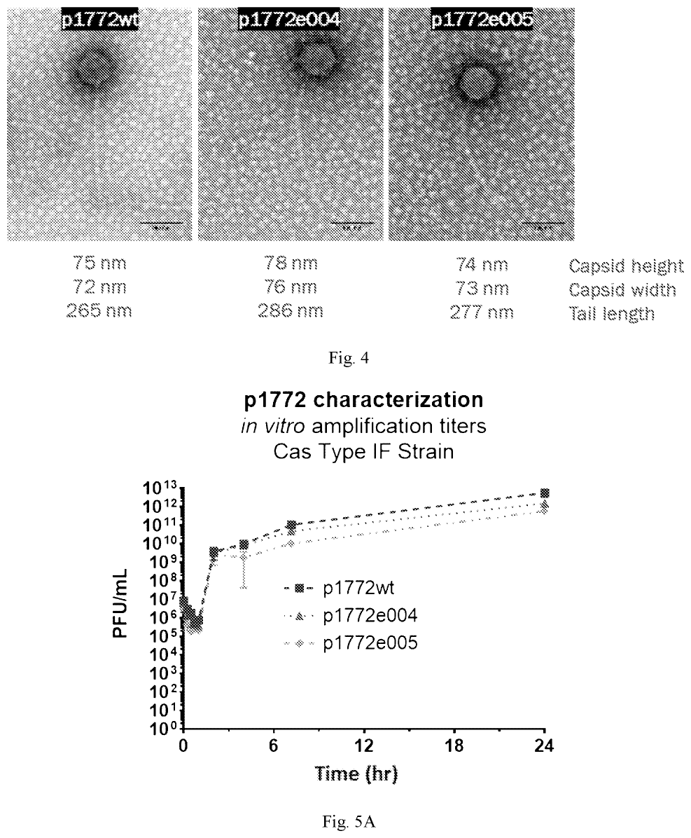

1.5 mL of crude lysate was centrifuged for 1 hr at 4° C. and 24,000×g. A fraction of the supernatant (approximately 1.4 mL) was gently discarded, and 1 mL of ammonium acetate (0.1 M, pH 7.5) was added to the remaining lysate, which was then centrifuged. This step was performed twice. Washed phage samples were visualized by negative-stain transmission electron microscopy. A glow-discharged formvar/carbon-coated 400 mesh copper grid (Ted Pella, Inc., Redding, Calif.) was floated on a 25-4, droplet of the sample suspension for five min, transferred quickly to two drops of deionized water followed by a droplet of 2% aqueous uranyl acetate stain for 30 sec. The grid was blotted with filter paper and air-dried. Samples were observed using a JEOL JEM-1230 transmission electron microscope operating at 80 kV and images were taken using a Gatan Orius SC1000 CCD camera with Gatan Microscopy Suite 3.0 software. Results are exemplified in FIG. 4. There were no apparent observable changes in phage morphology after modification.

### Example 5. Amplification of Phage Engineered with CRISPR-Cas Full Construct

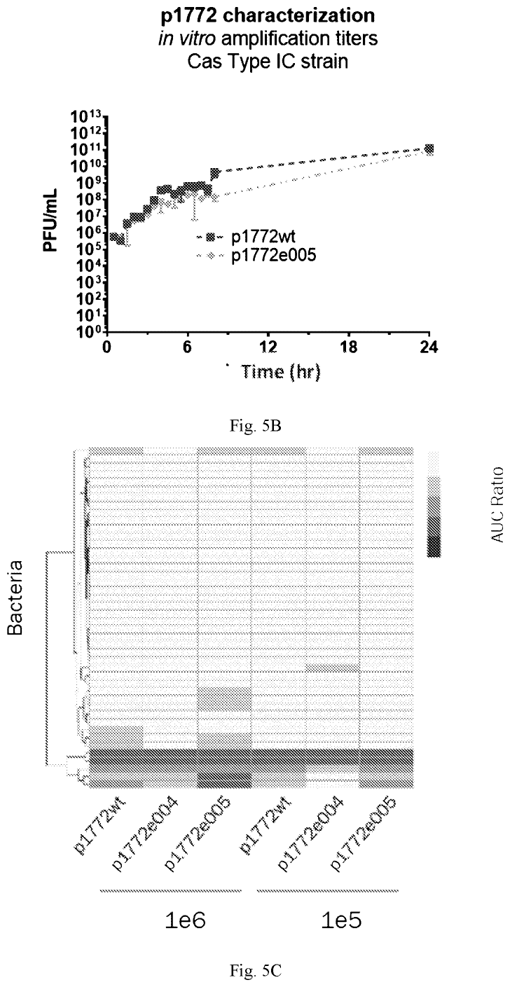

p1772 wt (wild type) and engineered variants p1772e004 (Cas system only) and p1772e005 (targeting crArray1+Cas system) were mixed with an exponentially growing culture of b1126 at a multiplicity of infection (MOI) of 1. At 0 min, 15 min, 30 min, 1 h, 2 h, 4 h, 7 h 10 min, and 24 h after infection, samples were collected for plaque forming units (PFU) enumeration (FIG. 5A-5B) and RNA isolation and quantification. For PFU enumeration, the samples collected at each time point were filtered through 0.45 μm filters to separate the phage from the host bacteria. A soft agar overlay was prepared as described for slide 3. 10-fold serial dilutions of the phage samples were spotted onto the overlay and incubated at 37° C. The following day, plaques were counted and used to calculate the PFU/mL in the initial sample. Based on these data, no significant differences in phage growth patterns were observed and each phage reached a similar maximum titer.

p1772 wt, p1772e004, and p1772e005 were diluted to a particle count of 1e6, and each individual phage was used to infect a panel of 34 different bacteria at an MOI of 0.01. Optical Density (OD) readings at a wavelength of 600 nm were captured every hour over a 20 hour time course. The resulting OD readings were used to generate bacterial growth curves in the presence of one of the three phages. Integration was used to calculate the Area Under the Curve (AUC) for each growth curve, where a smaller AUC upon phage addition indicates reduced bacterial load. Host range was determined by monitoring the OD600 (turbidity) of the culture over time to obtain a bacterial growth curve with the starting amount of introduced phage indicated on the bottom of the graph (input phage titer in plaque forming units per milliliter). The AUC for a given strain was compared in the presence and absence of phage. FIG. 5C exemplifies the AUC Ratio, in which the AUC calculation of strain growth in the presence of phages is divided by the AUC of strain growth in the absence of phage. Each row represents a unique bacterial strain. Darker values in the heatmap indicate stronger reductions in bacterial loads. The phage was considered to infect a given strain if (AUC in the presence of phage)/(AUC in the absence of phage) was less than 0.65. The heat map of AUC ratio demonstrates that the engineered phage variants had comparable host range to the wild type parent in this assay. The host range of p1772 wt, p1772e004, and p1772e005 were similar to one another and were within error of the assay. Host range confirmation of AUC hits by plaquing shows no difference between WT and CRISPR-Cas3.

Table 2 shows data from a representative growth experiment of two unique full construct-containing engineered phages pArray3 (targeting crArray3+Cas system) and pArray4 (targeting crArray4+Cas system). In this assay, an amplification was performed by inoculating LB growth medium with a single colony of bacteria and adding phage as indicated in the “Input PFU/mL” column. Amplifications were incubated overnight. Following incubation, the bacteria were removed by filtration and phage titer in the lysate was quantified by the soft agar overlay method. The titer of the lysate is indicated in the “Output PFU/mL” column. These data indicate that the engineered phages replicated effectively. These data also demonstrate the relative precision of the titration assay.

TABLE 2
Growth of pArray3 and pArray4

| | | Input | Output | Fold |
|---|---|---|---|---|
| Phage name | Replicate | PFU/ml | PFU/ml | increase |
| pArray3 | 1 | 3.00e+6 | 2.25e+9 | 750 |
| pArray3 | 2 | 1.90e+6 | 4.00e+9 | 2105 |
| pArray4 | 1 | 1.00e+5 | 8.00e+9 | 80000 |
| pArray4 | 2 | 5.00e+5 | 2.50e+9 | 5000 |

### Example 6. CRISPR-Cas System Expression in the Engineered Phage

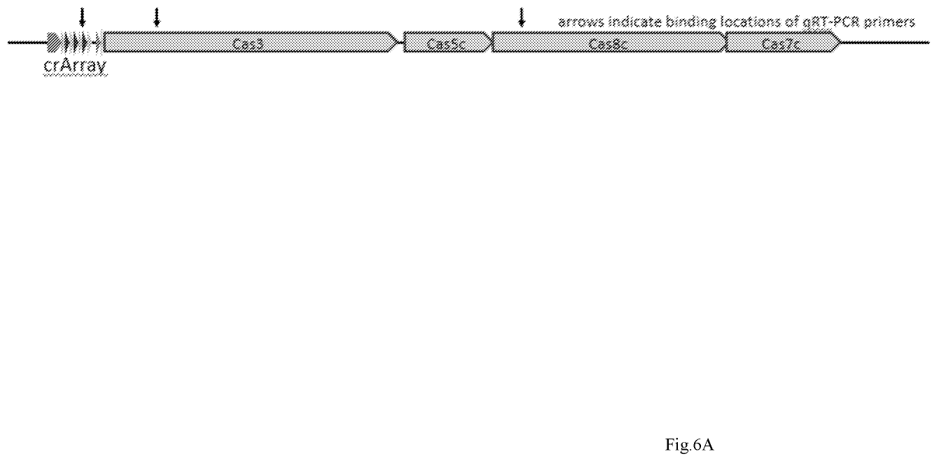

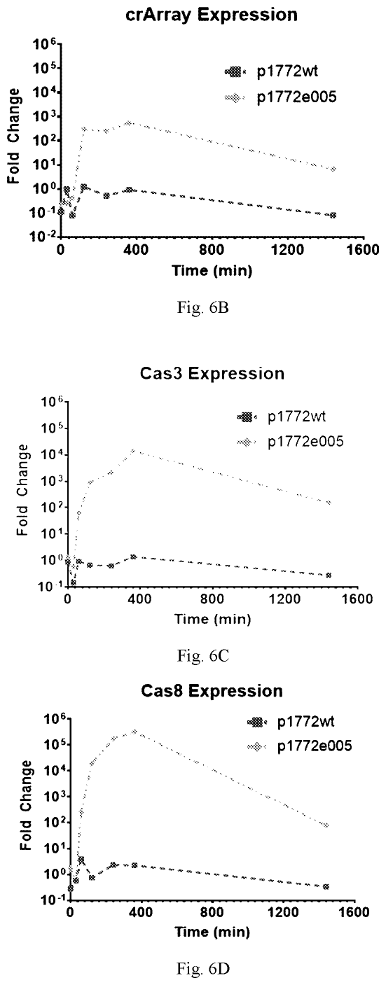

This example shows that the Cas system and a crArray was successfully expressed from the phage genome. FIG. 6A depicts the arrangement of the spacer array (crArray) and Cas operon that are engineered into p1772 and other phages described herein. Arrows represent the binding locations of primers pairs used for quantitative reverse transcription PCR (qRT-PCR) analysis of gene expression.

p1772 wt (wild type), which does not contain the Cas operon, was used as a control. For RNA isolation, the samples collected at each time point were added directly to RNAprotect. Samples were incubated for 5 minutes at room temperature, centrifuged for 10 minutes and 5000×g, and the supernatant discarded. Pellets were stored at −80° C. RNA was then isolated using the Qiagen RNeasy Mini Kit. cDNA was synthesized using the BioRad iScript cDNA synthesis kit. qRT-PCR was performed using BioRad SsoAdvanced Universal SYBR Green Supermix. All data was the average of two biological replicates. Fold change was 2^{-ΔΔCt}, using Pseudomonas aeruginosa gene rpsH as the housekeeping gene and comparing each data point to the cells only control at the same time point.

FIG. 6B-6D show relative expression levels of the indicated RNA following infection of a P. aeruginosa strain by different variants of phage p1772. The data in these graphs are presented as the fold change in expression compared to a vehicle control with no phage present. Each time point was normalized to the uninfected control for that time point. Changes in bacterial concentration were accounted for by normalizing the samples using the bacterial housekeeping gene rpsH. These data indicate that the phage produces crArray, Cas3, and Cas8c transcripts while infecting P. aeruginosa. The bacterial host used in panels B-D contained an endogenous Cas operon, so the difference between p1772e005 (targeting crArray1+Cas system) and p1772 wt represents the phage-mediated increase in expression over endogenous expression.

FIG. 6E shows the relative expression of cas3 mRNA from different engineered phage genomes. These data indicated that there is more cas3 expression from p1772e005 than from two other engineered phages, p2131e002 (targeting crArray1+Cas system) and p2132e002 (targeting crArray1+Cas system), at 1 h post-infection. However, at 24h post-infection, the phages expressed close to the same amount of cas3. These data were calculated by comparing cas3 RNA expression to the amount of phage gDNA and normalizing to p1772e005 at 1 h post infection. The bacterial strain used in these assays was Cas null, so there is no contribution from endogenous cas3 expression.

### Example 7. Phage Lytic Activity when Engineered with CRISPR-Cas Full Construct

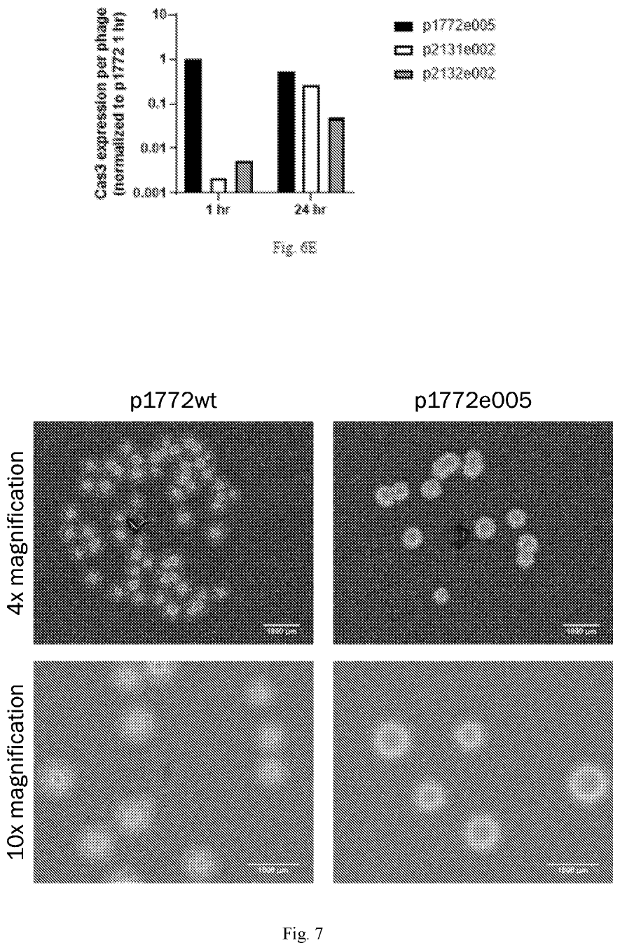

Top agar overlays were prepared by mixing 100 μL of a saturated overnight culture of the p1772 indicator strain b1121 with 6 mL of 0.375% agar in LB containing 10 mM MgCl₂ and 10 mM CaCl₂. After the top agar solidified, 2 μL drops of serial 10-fold dilution series of p1772 wt (wild type) and p1772e004 (Cas system only) and p1772e005 (targeting crArray1+Cas system) were spotted onto the surface of the top agar. Plates were incubated at 37° C. for ˜18 h, then imaged using a Keyence BZ-X800 microscope at 4× and 10× magnification. FIG. 7 illustrates the improved plaque morphology of p1772 phage. The morphology of the wild-type phage was observed to produce hazy plaques, while the engineered variant p1772e005 produced plaques of a similar size that have a hazy halo but are clear in the center. This data suggests that p1772e005 killed the bacteria more completely than p1772 wt.

### Example 8: Phage Containing the crArray and the Cas Operon were More Effective in Killing Bacteria than Phage Containing Only the crArray

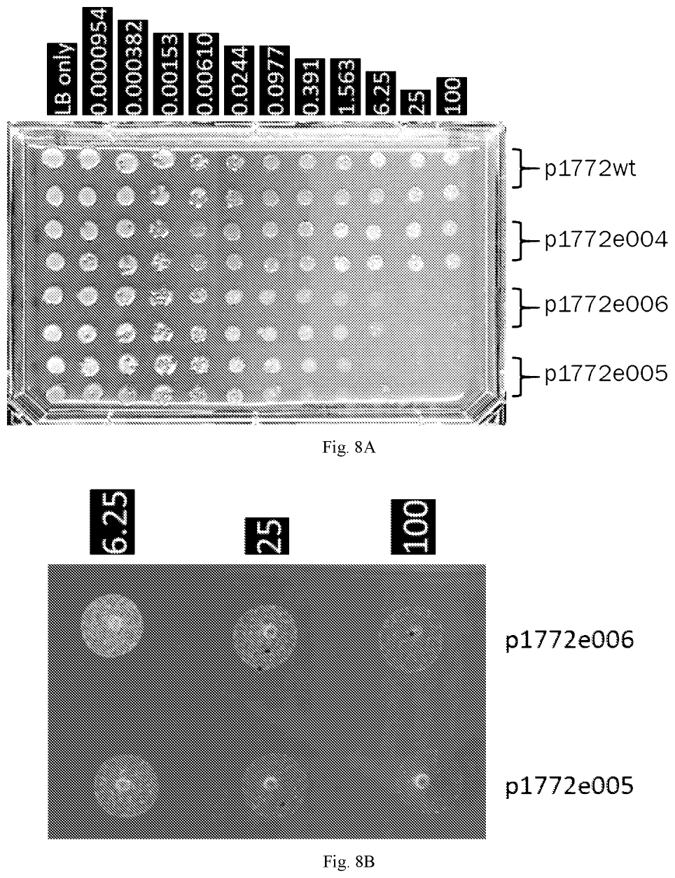

p1772 wildtype and engineered phage were mixed with bacteria in logarithmic growth and plated immediately in 2 ul spots on LB agar. The ratio of phage to bacteria was altered through the dilution series so that the amount of bacteria stays constant at each dilution but the amount of phage was a 1 to 4 dilution. At the highest dilution, the multiplicity of infection (MOI) was 100, meaning there were approximately 100 phages per bacteria. In FIG. 8A, both p1772e005 (targeting crArray1+Cas system), and p1772e006 (targeting crArray1 only) consistently killed the majority of the bacteria present in Type I-C strains after overnight incubation, as indicated by little to no bacterial colonies that grew in those spots whereas the wildtype phage did not control bacterial colony formation. Thus, wildtype and p1772e004 (Cas system only) were unable to control bacterial replication, even at an MOI of 100. FIG. 8B shows that p1772e006 killed bacteria more effectively than wildtype in this bacteria strain due to the endogenous Cas system in the bacteria, however it did not appear to be as effective as phage that also contains an exogenous Cas system (p1772e005). This was because with extended incubation time, the more bacteria form colonies in spots exposed to p1772e006 than p1772e005.

FIG. 8C is a quantification of a single MOI from the same type of assay performed in FIGS. 8A-8B. In contrast to FIGS. 8AB-8B, the bacterial strain in panel C did not have an endogenous Cas system but had a genomically integrated copy of the mCherry gene. The plate was imaged and the fluorescence of each spot was quantified. The results for an MOI of 1.5 are shown, but MOIs above 0.4 all have results consistent with the MOI of 1.5. Due to the lack of an endogenous Cas system, the crArray-only phage (p1772e006) behaved similarly to the wild type phage. The fully engineered phage containing a non-targeting crArray (p1772e008) was also not improved relative wild type. However, the fully engineered phage containing a targeting crArray (p1772e005) inhibited cell growth to a significantly greater extent than any other phage variant. This data shows that the fully engineered variant did not require an endogenous Cas system to be effective.

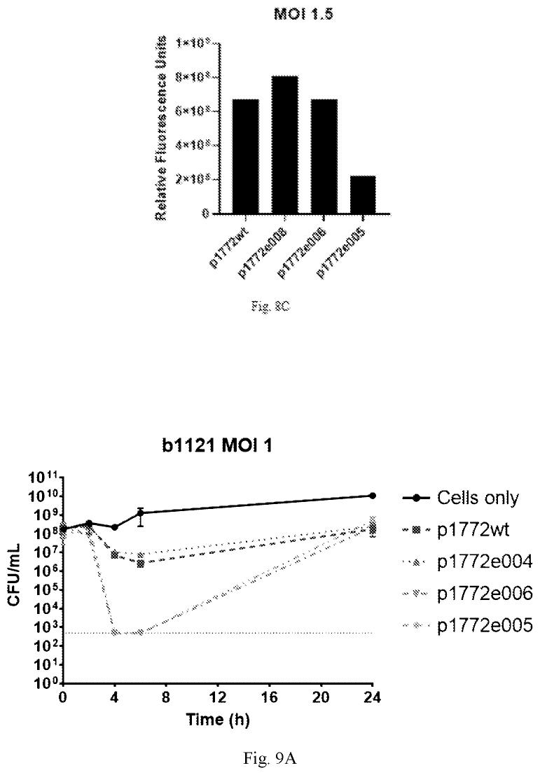

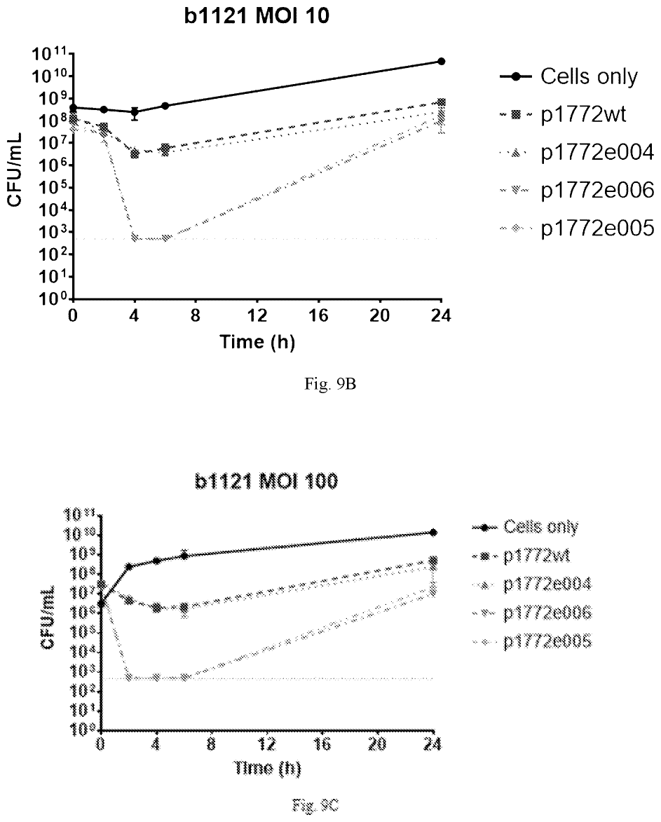

A P. aeruginosa strain (b1121) with an active endogenous Type I-C Cas system was grown to mid-logarithmic phase and infected with phage in liquid culture at the indicated multiplicity of infection (MOI). In all cases, the phage successfully killed the bacteria, as depicted in FIGS. 9A-9C, indicated by a reduction in colony forming units (CFU)/mL recovered compared to an uninfected control. Both p1772e005 (targeting crArray1+Cas system) and p1772e006 (targeting crArray1 only) killed the bacteria more efficiently than the wild type phage. p1772e004 (Cas system only) did not have improved activity relative to p1772 wt (wild type) or the self-targeting variants, demonstrating that both the self-targeting crRNA and Type I CRISPR-Cas components were required to improve phage efficacy. Notably, p1772e006 and p1772e005 killed to equal levels, demonstrating that the engineered phage variants were able to kill by expression of bacterial-targeting Cas systems from the phage in the presence of a compatible and active Cas system. The dotted lines in these figures represents the limit of detection (LOD) for the assay. Samples for which no colonies were obtained are shown at the LOD.

### Example 9: Multiple Different P. aeruginosa Targeting Spacers Improved Phage Efficacy

p1772 wildtype and engineered phage variants were mixed with mCherry expressing bacteria in logarithmic growth and plated immediately onto LB agar. The ratio of phage to bacteria was altered by performing a dilution series of the phage, so that the amount of bacteria remained constant in each spot but the amount of phage changed. The highest multiplicity of infection (MOI) was 100, meaning there are approximately 100 phages per bacterium. After overnight incubation, bacterial growth was recorded by imaging the plate by brightfield and mCherry fluorescence. Quantification was performed on the samples based on these images.

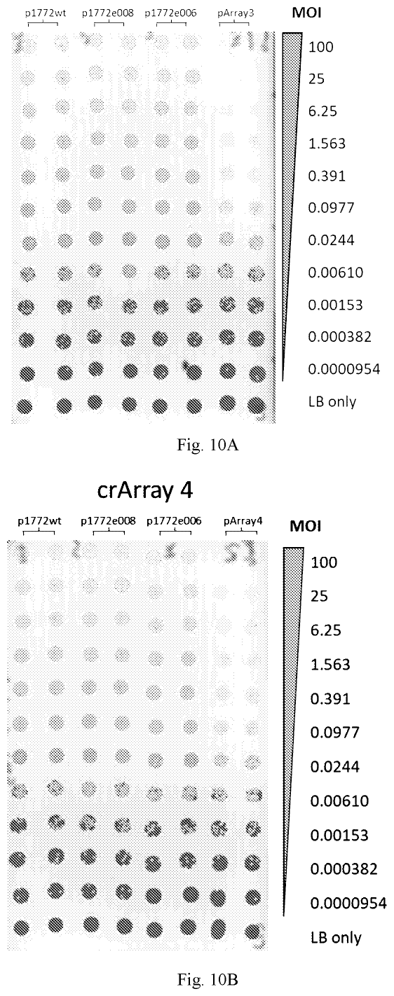

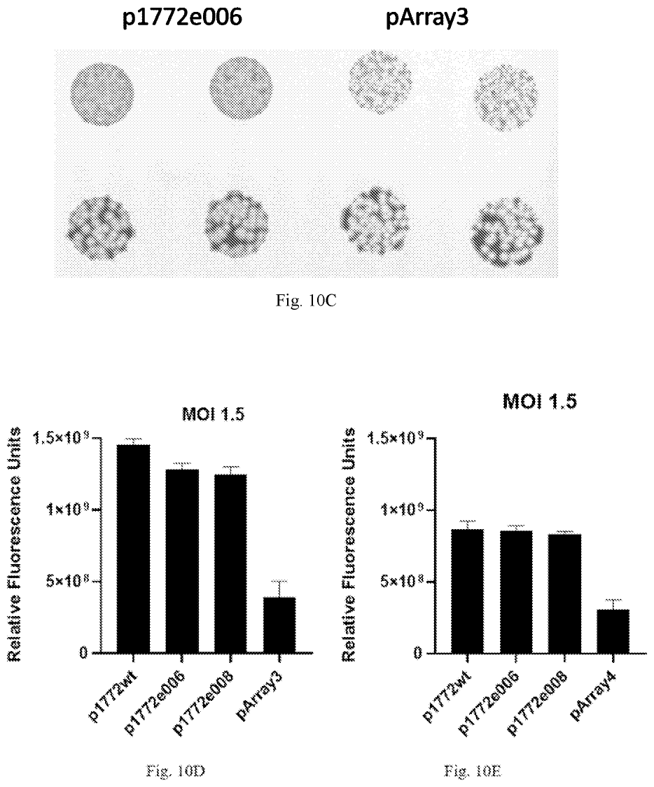

Five different phage variants were used to determine the effect of lytic phage delivering non-targeting crRNAs with an exogenous Type I-C Cas system (p1772e008), a self-targeting crRNA alone without an exogenous CRISPR-Cas system (p1772e006), two different self-targeting crArrays delivered with an exogenous Type I-C Cas system (pArray3 and pArray4) and the parent wild-type phage (p1772 wt). In these assays, a P. aeruginosa strain lacking any endogenous Cas system and the indicated phage were combined at the indicated ratio and immediately plated on LB plates. The host bacterial strain used in these assays was Cas-null and had a chromosomally integrated mCherry gene to facilitate observation and quantification of bacteria through measurement of relative fluorescence. The results are depicted in FIGS. 10A-10B. Darker spots represent higher bacterial growth. The numbers to the right of each image represent the multiplicity of infection (MOI). At the highest MOI, there were approximately 100 phage for every 1 bacterium. These plate images show that at higher MOIs, phages pArray3 and pArray4 (both p1772 phage encoding an active Type I-C Cas system and each with a unique crArray composed of three distinct self-targeting spacers each) killed P. aeruginosa more effectively than p1772 wt, the phage with the Cas operon and non-targeting spacers (p1772e008), or p1772 containing the crArray but no exogenous Cas system (p1772e006). As expected, the crRNA only phage (p1772e006) did not improve phage efficacy as the bacteria do not have an endogenous Cas system.

FIG. 10C is a higher resolution view of the box in FIG. 10A, and highlights the differences between the fully engineered phage (pArray3) and a phage with a crArray only and no Cas operon (p1772e006). In the bottom row (MOI 0.00610), pArray3 formed clearer plaques than did p1772e006 (i.e., the light spots in the pArray3 samples were lighter). In the top row (MOI 0.0244), pArray3 inhibited bacterial growth (dark spots) better than p1772e006.

FIG. 10D-E show the quantification of the fourth row down (MOI ˜1.5) of the corresponding fluorescent images of the same plates shown in FIG. 10A and FIG. 10B that quantify the relative amount of fluorescent bacteria present. Consistent with the brightfield images, at an MOI of about 1.5, samples treated with pArray3 and pArray4 had significantly less fluorescent signal (indicating loss of viable bacterial cells) than samples treated with wildtype phage or the non-targeting (p1772e008) and crArray-only (p1772e006) engineered phages.

### Example 10: Efficacy of the crArray/Cas Insert with Different Promoters Driving Expression of the Cas Operon

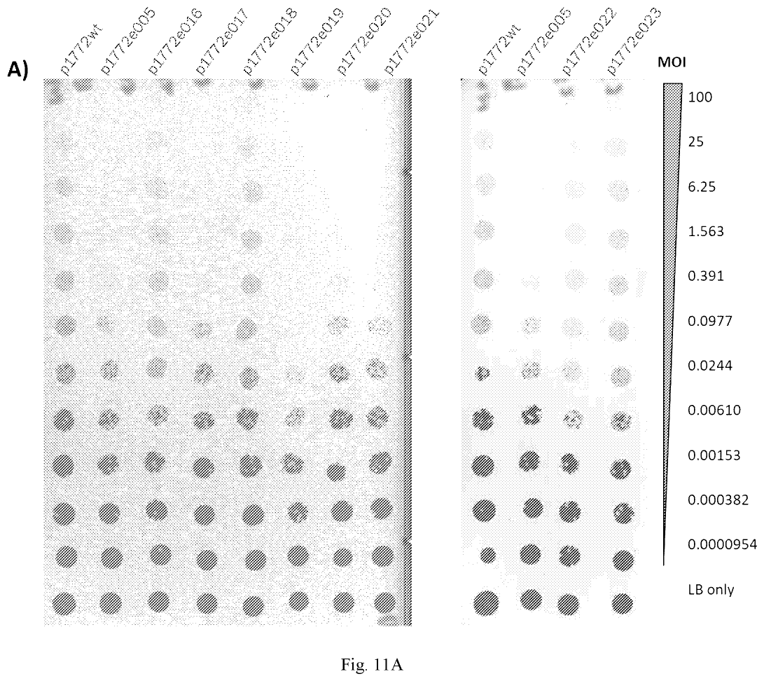

p1772 wildtype and engineered phage variants were mixed with mCherry expressing bacteria in logarithmic growth and plated immediately onto LB agar. The ratio of phage to bacteria was altered by performing a dilution series of the phage, so that the amount of bacteria remained constant in each spot but the amount of phage changed. The highest multiplicity of infection (MOI) was 100, meaning there were approximately 100 phages per bacterium. After overnight incubation, bacterial growth was recorded by imaging the plate by brightfield and mCherry fluorescence. Quantification was performed on the samples based on these images. FIG. 11A shows bacterial kill by p1772 wild type and multiple engineered variants of the phage containing the Cas system and crArray expressed by different promoters. All of the engineered phage variants had the same structure as p1772e005 (see FIG. 3A) and differ only in the identity of the promoter driving expression of the Cas operon. p1772e016 used the promoter that drives the endogenous Type I-C Cas system in P. aeruginosa. p1772e005, p1772e017, p1772e021 all used E. coli promoters or derivatives of E. coli bacterial promoters. p1772e018, p1772e022, and p1772e023 all used P. aeruginosa bacterial promoters. p1772e019 and p1772e020 used P. aeruginosa phage promoters. Both plates were from the same assay and controls (p1772 wt and p1772e005) are from the same phage-bacteria mixture prior to plating the spots. The bacterial host strain used was a Cas-null P. aeruginosa strain that expressed mCherry from the chromosome. Individual images were acquired using a 4× objective and brightfield illumination, then stitched together to obtain the images shown here. FIG. 11B shows the quantification of the fourth row down (MOI ˜1.5) of the corresponding fluorescent images of the same plates shown in FIG. 11A. Differences in overall efficacy were observed across different promoters used, indicated by significantly less fluorescent signal (indicating loss of viable bacterial cells) compared to p1772 wt.

### Example 11: Multiple Different Phages have Improved Efficacy for Log Reduction

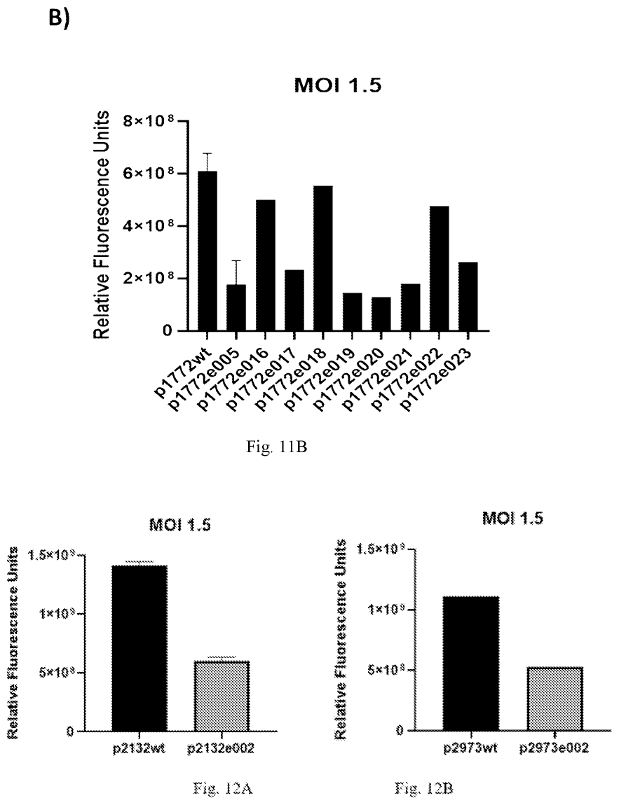

Wildtype and engineered phage variants were mixed with mCherry expressing bacteria in logarithmic growth and plated immediately onto LB agar. The results shown are from a multiplicity of infection (MOI) of 1.5, meaning there were approximately 1.5 phages per single bacterium. After overnight incubation, bacterial growth was recorded by imaging the plate for mCherry fluorescence. Quantification as depicted in FIGS. 12A-12B was performed on the samples based on these images. Two unique wildtype phages (p2131 and p2973) and their engineered counterparts containing the Cas system and crArray 1 (p2132e002 and p2973e002) were tested. At an MOI of about 1.5, the engineered phage had far less viable bacteria than wildtype phage. These results show that the phage-delivered Cas system works in multiple unique phages.

### Example 12: The Efficacy of the Spacer Array/Cas Insert in Alternative Phages and Pseudomonas Strains

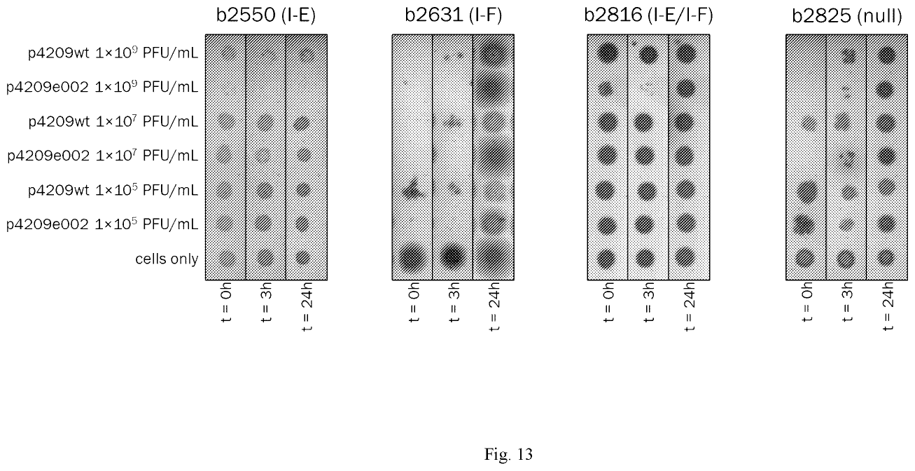

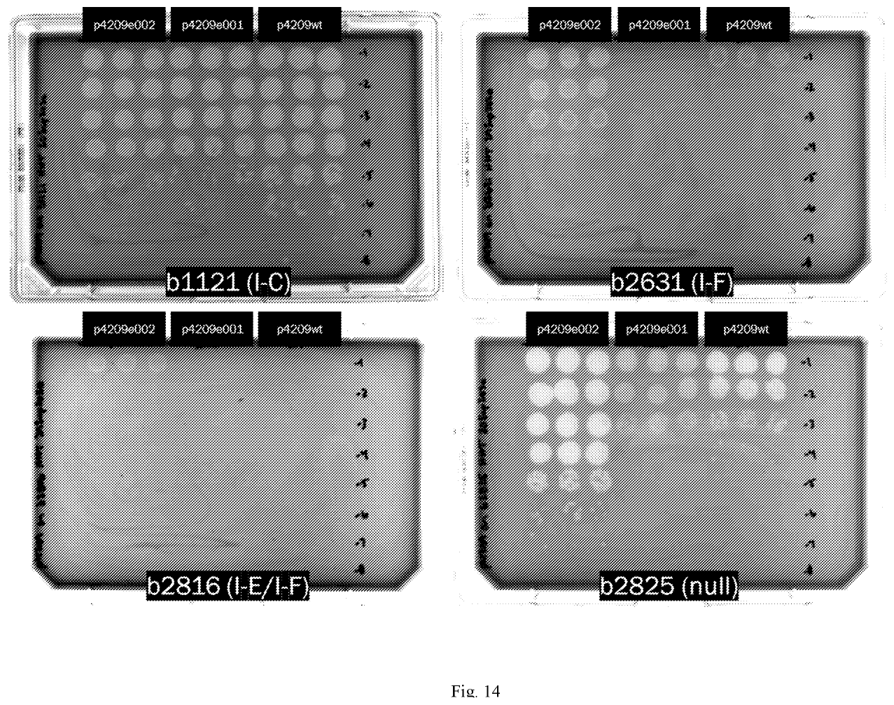

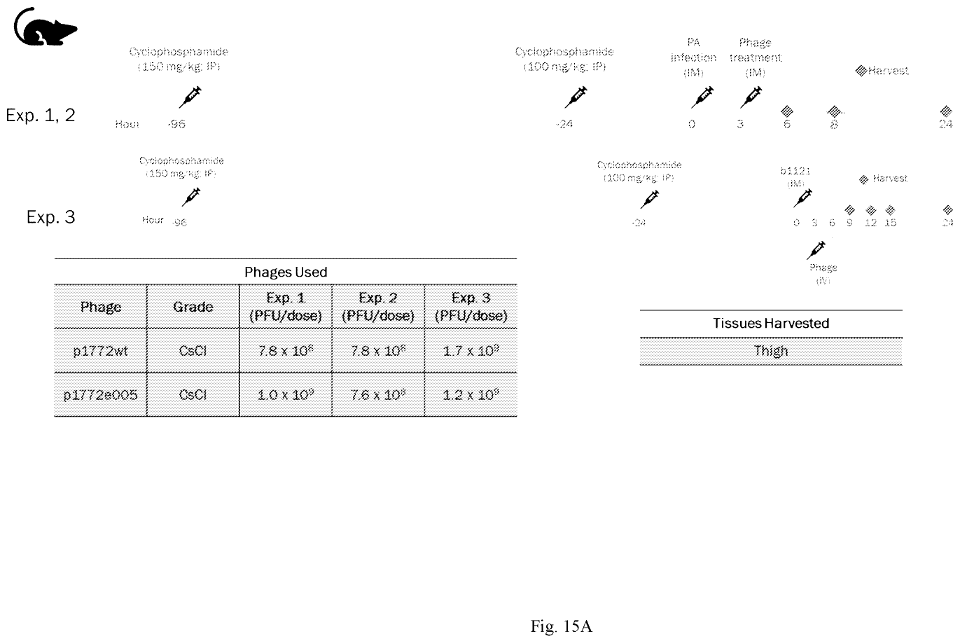

An was performed with p4209 wt (wild type) and p4209e002 (targeting crArray1+Cas system) against a panel of Pseudomonas strains. Briefly, early log phase bacterial culture was mixed with phage to obtain the final titers listed in the figure. Samples were plated immediately (t=0 h) and after 3 and 24 h of incubation at 37° C. Plates were imaged and differences between the wild type and full construct variants tabulated. p4209 wildtype and engineered phage variants were mixed with bacteria in logarithmic growth and plated immediately onto LB agar, or incubated in liquid for the indicated amount of time before plating. The ratio of phage to bacteria was altered by performing a dilution series of the phage, so that the amount of bacteria stays constant in each spot but the amount of phage changes. The relative ratio of the phage and bacteria shifted over the course of the experiment as the bacteria replicated and succumbed to the phage. After overnight incubation, bacterial growth was recorded by imaging the plate. The label at the top of each set of images denotes the Cas type of the bacterial strain shown in that image. FIG. 13A shows the results of this assay. In strain b2550 at all timepoints, p4209e002 fully inhibited bacterial growth at a titer of 1×10⁹ PFU/mL compared to p4209 wt at the same titer. In strain b2631 at t=0 h, no growth was observed for p4209e002 at a titer of 1×10⁵ PFU/mL while growth was visibly greater for p4209 wt at the same titer. In the same strain at t=3 h, no growth was observed for p4209e002 at any titer while there was visible growth for p4209 wt at all titers. In strain b2816 at t=0 h, slightly less growth was observed for p4209e002 at a titer of 1×10⁹ PFU/mL than for p4209 wt at the same titer. In the same strain at t=3h, very little growth was observed for p4209e002 at a titer of 1×10⁹ PFU/mL while there was significant growth for p4209 wt at the same titer. In strain b2825 at t=0 h, no growth was observed for p4209e002 at a titer of 1×10⁷ PFU/mL while significant growth as observed for p4209 wt at the same titer. In the same strain at t=3 h, some growth was observed for p4209e002 at titers of 1×10⁹ PFU/mL and 1×10⁷ PFU/mL, while there was visibly more growth for p4209 wt at the same titers. Taken together, these data show that a unique phage, p4209e002, had improved Cas and crRNA spacer activity against several unrelated P. aeruginosa strains.

p4209 wt, p4209e001 (Cas system only), and p4209e002 (targeting crArray1+Cas system) were plagued on multiple bacterial strains to examine the efficiency of plaquing. P. aeruginosa strain b1121 supported all variants equivalently and is provided as a titer reference. On P. aeruginosa strain b2631, the wild type variant plagued at a significantly decreased level, the Cas-only variant did not plaque at all, and the fully engineered variant plagued with no loss of efficiency compared to b1121. On P. aeruginosa strain b2816, neither the wild type nor Cas-only variants showed any evidence of activity, while the fully engineered variant produced zones of clearing. On P. aeruginosa strain b2825, the wild type and Cas only variants had significantly reduced plaquing efficiency, while the fully engineered variant maintained comparable efficiency to b1121. Both b2631 and b2825 show examples of an engineering event (insertion of the Cas system) having detrimental effects—that is, either a decrease in efficiency of plaquing (b2631) or in plaque clarity (b2825). In both cases, addition of the targeting crArray (which enables activity of the Cas system) not only rescued the decreased activity but improved activity beyond that seen in the wild type parent. The label at the bottom of plate image denotes the bacterial strain shown in that image and the type of endogenous Cas system it contains. These results further support that the Cas system and targeting crArray improved the phages ability to replicate and kill various bacterial strains.

### Example 13. In Vivo Efficacy Study

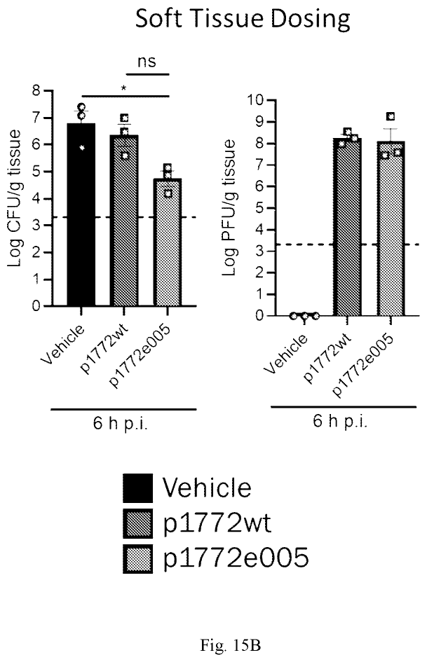

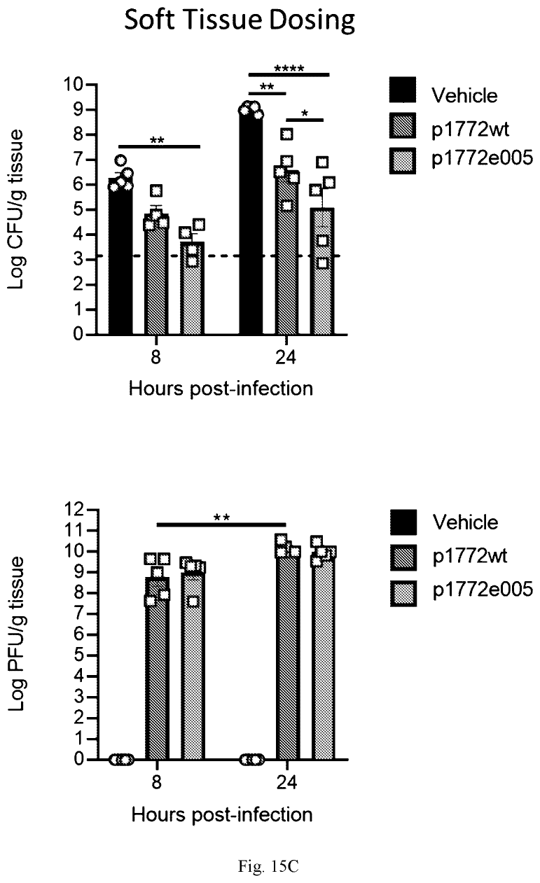

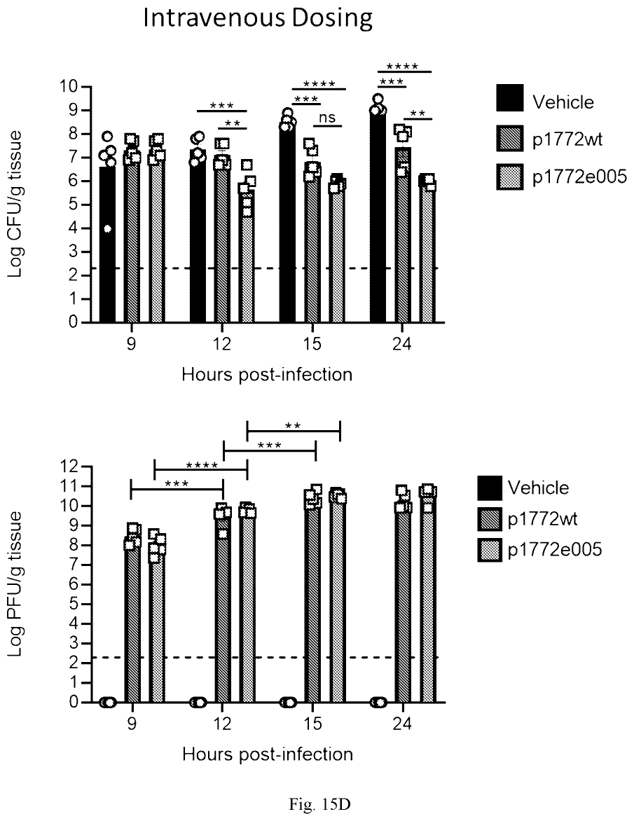

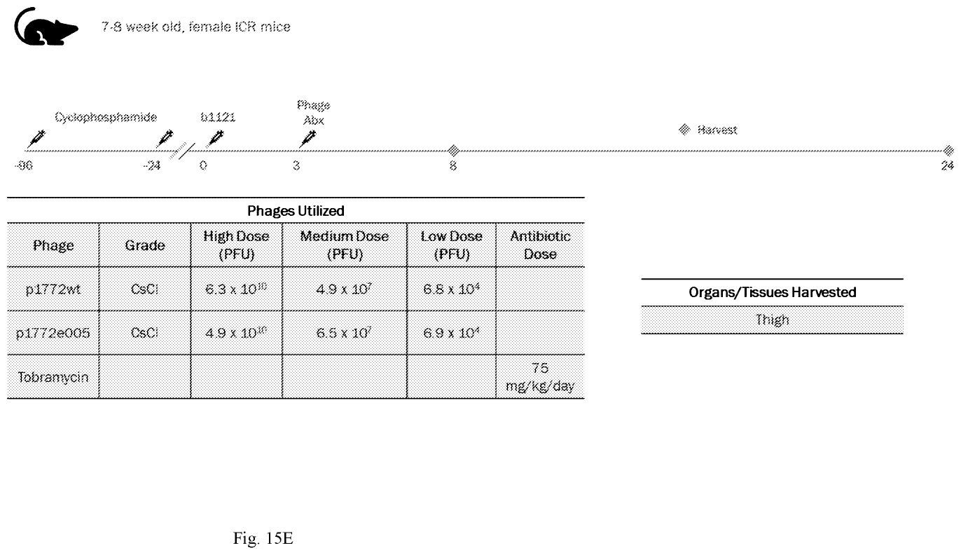

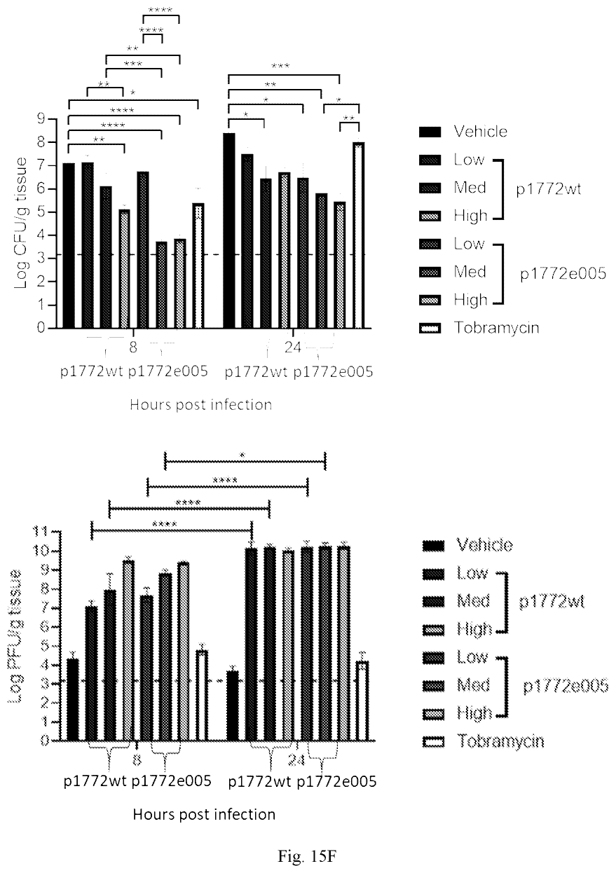

FIG. 15A outlines the materials and methods utilized for in vivo efficacy modeling with p1772 wt (wild type) and p1772e005 (targeting crArray1+Cas system). Female, ICR mice from Envigo were rendered neutropenic via two intraperitoneal injections of cyclophosphamide (150 mg/kg and 100 mg/kg, respectively) on Days −4 and −1. Following induction of neutropenia, mice were infected with P. aeruginosa b1121 by a single intramuscular injection. Previous model development determined that ˜5e6 CFU was the ideal inoculum of this particular strain. At 3 h post-infection (p.i), mice were treated with either vehicle (1×TBS+10 mM salts), p1772 wt, or p1772e005 by a single intramuscular injection in the infected thigh. The table on the bottom left details the total PFU delivered to each infected thigh in each experiment. Mice were euthanized and the thigh muscle harvested at the indicated time point post-inoculation. The thighs were homogenized using a bead beater system. Homogenate was serially diluted and plated for CFU quantification. Homogenate was also filtered through a 0.45 um filter. Filtrate was serially diluted and plated for PFU quantification on a b1121 overlay plate. All CFU and PFU measurements were normalized to g tissue.

Both the bacterial colony forming units (CFU) and phage plaque forming units (PFU) are shown for each experiment. FIG. 15B-15C show phage efficacy in mice where the phage were administered intramuscularly. Thigh muscle tissue was harvested at the indicated time points. Both replicates show that the fully engineered phage decreases colonization to a greater extent than does the wild type phage. FIG. 15D shows phage efficacy in mice where the phage were administered intravenously. Thigh muscle tissue was harvested at the indicated time points. The fully engineered phage destroys the bacteria to a greater extent than does the wild type phage. Taken together, these data from FIGS. 15B-15D indicate that phage delivered by different routes enters the thigh and kills the bacteria. At every time point, the CFU/g of thigh tissue in mice treated with fully engineered phage is lower than that in mice treated with the wildtype phage. FIG. 15E is a schematic showing the experimental design for a model establishing the dose-response of phage treatment in a mouse infection model. This experiment was performed similarly those shown in FIG. 15A, but additionally includes an antibiotic treatment group to represent the current standard of care. The phage dose is also titrated between the different groups.

FIG. 15F shows the results of treatment with phage at different doses or antibiotic in mice. Overall, these data indicate that the engineered p1772e005 was more effective in a mouse infection model than p1772 wt. Moreover, the engineered phage performed better than the antibiotic treatment given. In panels B-D and F, data is shown as mean±SEM. *p<0.05, **p<0.01, ***p<0.001, ****p<0.0001. Statistical significance is determined using a One-way ANOVA with multiple comparisons or Two-way ANOVA with Tukey's test.

### Example 14: Targeting crArrays in E. coli

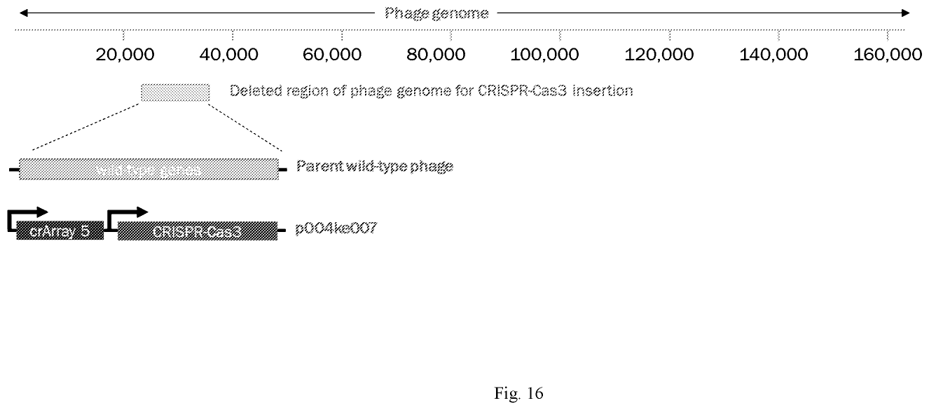

FIG. 16 shows a schematic representation of the genome of wild type phage p004k and its engineered variant p004ke007. The bar below the genome axis indicates the region of the genome that was removed and replaced. The schematic below the phage genome illustrates the DNA that was used to replace WT phage genes in the deleted region.

E. coli phage p004k-wt (wild type) and p004ke007 (targeting crArray5+Cas system) were mixed with the indicated bacterial strain while the bacteria was in logarithmic growth and plated onto LB agar 3 hours post inoculation. The ratio of phage to bacteria was altered by performing a dilution series of the phage, so that the amount of bacteria remained constant in each spot but the amount of phage changed. The relative ratio of the phage and bacteria shifted over the course of the experiment as the bacteria replicate and succumb to the phage, which is why an MOI is not listed. The label at the top of each set of images denotes the strain shown.

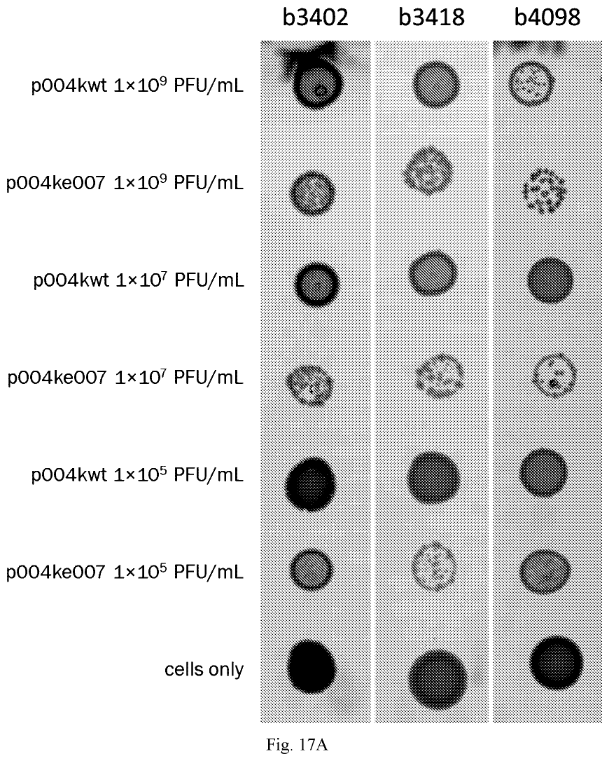

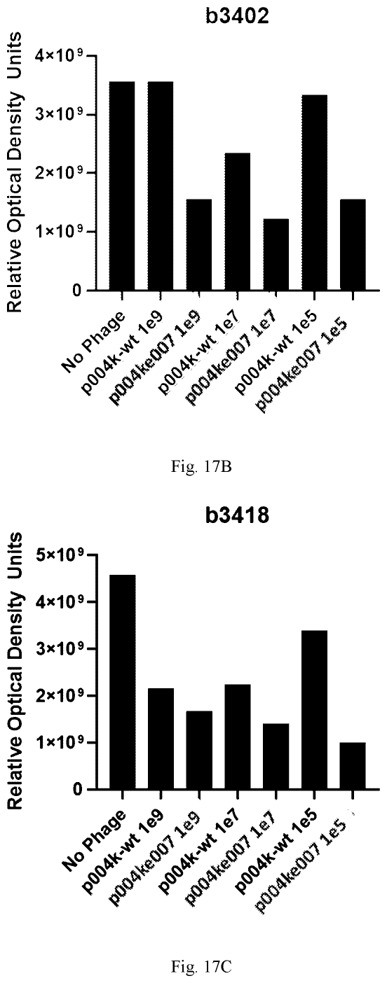

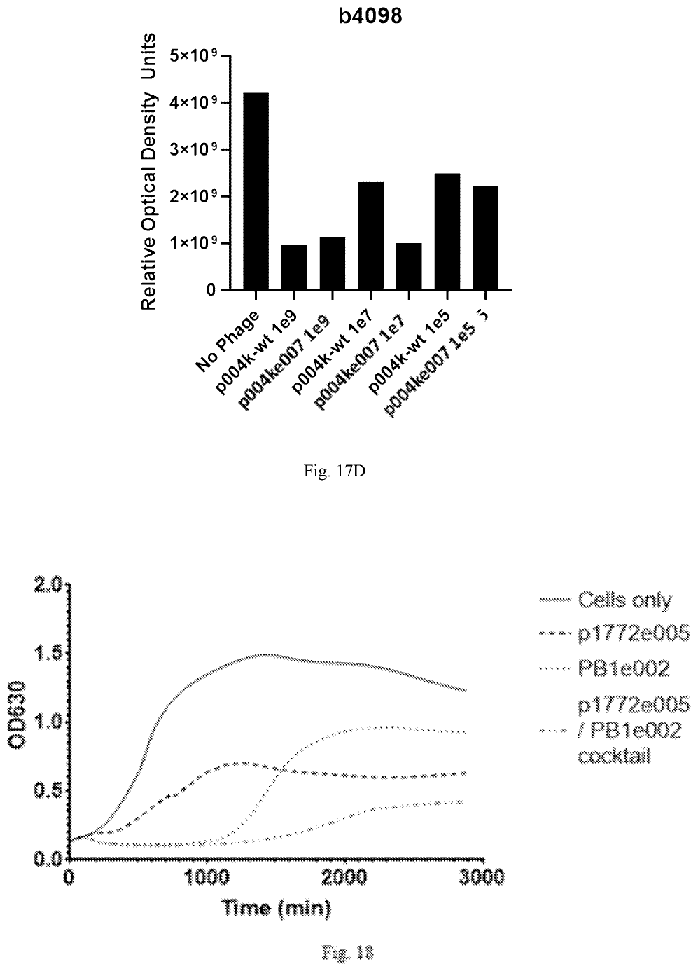

In this assay, the phage was mixed 1:1 with the indicated bacterial culture in mid-logarithmic growth to obtain the final phage titers listed on the left side of the image. The bacteria-phage mixture was incubated for 3 hours and then 2 ul of the culture was spotted onto LB plates, as depicted in FIG. 17A. Bacteria that survived the phage replicate to form visible colonies, so fewer bacteria means better phage kill. In this assay, the engineered phage appeared to kill the bacteria better than wildtype at all dilutions, but the 1×10⁷ dilution was the most visually striking for each E. coli strain. FIG. 17B-17D show quantification of the images in FIG. 17A. The quantification was determined by comparing the relative optical density of each spot (essentially how dark each spot is, with a darker spot being indicative of more cell growth). Together, these data show that the engineered phage containing a Cas systems and crArray targeting the bacterial genome has improved kill over the wild type phage parent in multiple strains. These data demonstrate that phage expressing exogenous Cas systems improve the phage against diverse human pathogens.

### Example 15. In Vivo Efficacy Study

A culture of b1121 was grown overnight, back diluted into LB+10 mM MgCl₂+10 mM CaCl₂ and grown to an OD600 of 0.45. The culture was split and treated with either LB/salts (cells only control), p1772e005 (MOI=0.1), PB1e002 (MOI=0.1), or a cocktail of p1772e005+PB1e002 (MOI=0.1 per phage). All samples were incubated in a microtiter plate at 37° C. with shaking for 24 h and the OD at 630 nm was measured every 10 minutes. Data is presented as a mean of 12 replicates. Error bars represent the standard deviation. The data show that the cocktail of the two full construct phages suppresses culture rebound to a greater extent than either phage by itself. FIG. 18A shows cooperativity between p1772e005 and PB1e002.

### Example 16: The Activity of Different Repeat Sequences

A culture of P. aeruginosa cultures were transformed with vectors comprising the different repeat sequences. The vectors were either an empty vector pUCP19 (empty vector) or contained an pUCP19 vector comprises a Pseudomonas Type I C Cas system and a spacer targeting the gyrB gene flanked by the repeat sequence listed in Table 3. An aliquot was taken of each test condition, diluted and spotted to enumerated bacterial CFUs.

TABLE3
Repeatsequences

| | SEQ | |
|---|---|---|
| | ID | |
| Repeat | NO: | Sequence |
| Repeat1 | 24 | GTCGCGCCCCGCACGGGCGCGTGGATTGAAAC |
| Repeat2 | 25 | GTCGCGCCCCGCACGGGCGCGTGGAGTGAAAG |
| Repeat3 | 26 | GTCGCGCCCCGCACGGGTGCGTGGATTGAAAC |
| Repeat4 | 27 | GTCGCGCCCCGCATGGGCGCGTGGATTGAACA |
| Repeat5 | 28 | GTCGCGCCCTACGCGGGCGCGTGGAGTGAAAG |

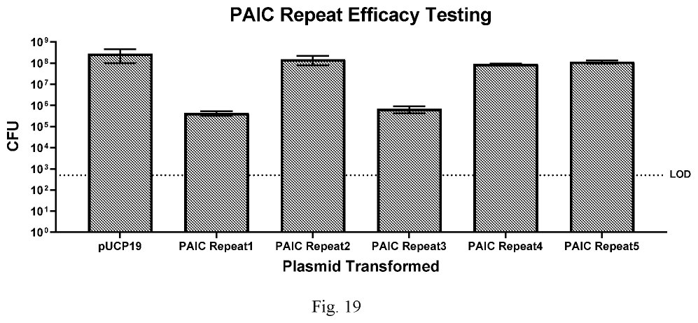

The results of this assay are depicted in FIG. 19. Specific sequences resulted in different number of transformants. Both repeat 1 and repeat 3 both resulted in a lower number of transformants than the empty vector or bacteria transformed with repeat 2, 4, or 5. This indicates that the sequence of the repeat sequence effects the efficacy of phage targeting in the Pseudomonas cultures.

### Example 17: Designing and Validating Spacer Sequences to Target a Target Bacterium Spacer Design

A spacer sequence is designed using the following protocol. First, suitable search set of representative genomes for the organism/species/target of interest are acquired. Examples of suitable databases include NCBI GenBank and the PATRIC (Pathosystems Resource Integration Center) database. The genomes are downloaded in bulk via FTP (File Transfer Protocol) servers, enabling rapid and programmatic dataset acquisition.

The genomes are searched with relevant parameters to locate suitable spacer sequences. The genomes can be read from start to end, in both the forward and reverse complement orientations, to locate contiguous stretches of DNA that contain a PAM (Protospacer Adjacent Motif) site. The spacer sequence will be the N-length DNA sequence 3′ adjacent to the PAM site, where N is specific to the Cas system of interest and is generally known ahead of time. Characterizing the PAM sequence and spacer sequences are generally performed during the discovery and initial research of a Cas system. Every observed PAM-adjacent spacer can be saved to a file and/or database for downstream use.

Next, the quality of a spacer for use in a CRISPR engineered phage is determined using the following process. First, each observed spacer can be evaluated to determine how many of the evaluated genomes they are present in. The observed spacers can additionally be evaluated to see how many times they may occur in each given genome. Spacers that occur in more than one location per genome can be advantageous because the Cas system may not be able to recognize the target site if a mutation occurs, and each additional “backup” site increases the likelihood that a suitable, non-mutated target location will be present. The observed spacers can be evaluated to determine whether they occur in functionally annotated regions of the genome. If such information is available, the functional annotations can be further evaluated to determine whether those regions of the genome are “essential” for the survival and function of the organism. Focusing on spacers that occur in all, or nearly all, evaluated genomes of interest (>=99) ensures broad applicability to justify the spacer selection. Provided a large selection pool of conserved spacers exists, preference may be given to spacers that occur in regions of the genome that have known function, with higher preference given if those genomic regions are “essential” for survival and occur more than 1 time per genome.

### Spacer Validation

The identified spacer sequences can then be validated by completing the following procedure. First, a plasmid that replicates in the organism(s) of interest and has a selectable marker (e.g. an antibiotic-resistance gene) is identified. The genes encoding the Cas system are inserted into the plasmid such that they will be expressed in the organism of interest. Upstream of the Cas system, a promoter is included that is recognized by the organism of interest to drive expression of the Cas system. Between the promoter and the Cas system, a ribosomal binding site (RBS) is included that is recognized by the organism of interest.

Next, genome-targeting spacers that have been identified bioinformatically are inserted into the plasmid that expresses the Cas system. Upstream of the repeat-spacer-repeat, promoter is included that is recognized by the organism of interest to drive expression of the crRNA. Examples of such promoters are listed in Table 1B. This cloning must be performed in an organism or strain that is not targeted by the spacer being cloned.

Next, a non-targeting spacer is inserted into the plasmid that expresses the Cas system. The sequence of this spacer can be randomly generated and then confirmed bioinformatically to not have targeting sites in the genome of the organism of interest. Upstream of the repeat-spacer-repeat, a promoter that is recognized by the organism of interest to drive expression of the crRNA is included.

Next, the killing efficacy of each tested spacer is determined. The plasmids listed in Table 4 are normalized to the same molar concentration. Each plasmid is transferred to the organism of interest by transformation, conjugation, or any other method for introducing a plasmid into a cell. The transformed cells are plated onto the appropriate selective media (e.g. antibiotic-containing agar). Following cell growth into colonies, the colonies that resulted from each different plasmid transfer are enumerated. Plasmids containing targeting spacers with a significantly lower transfer rate than the control plasmid containing the non-targeting spacer are considered to be successful at targeting the bacterial genome.

TABLE 4
Plasmids and controls used

| Plasmid | Function |
|---|---|
| Empty backbone vector | control for plasmid transfer efficiency |
| Vector containing the Cas system | control for Cas system toxicity |
| Vector containing the Cas system | control for off-target effects |
| and the non-targeting spacer | |
| Vector containing the Cas system | test sample |
| and the targeting spacer | |

While preferred embodiments of the present disclosures have been shown and described herein, it will be obvious to those skilled in the art that such embodiments are provided by way of example only. Numerous variations, changes, and substitutions will now occur to those skilled in the art without departing from the disclosures. It should be understood that various alternatives to the embodiments of the disclosures described herein may be employed in practicing the disclosures. It is intended that the following claims define the scope of the disclosures and that methods and structures within the scope of these claims and their equivalents be covered thereby.

What is claimed is:

1. A bacteriophage comprising a nucleic acid sequence encoding a Type I CRISPR-Cas system comprising:
(a) a CRISPR array;
(b) a Cascade polypeptide; and
(c) a Cas3 polypeptide.

2. The bacteriophage of claim 1, wherein the CRISPR array comprises a spacer sequence and at least one repeat sequence.

3. The bacteriophage of claim 2, wherein the at least one repeat sequence is operably linked to the spacer sequence at either its 5′ end or its 3′ end.

4. The bacteriophage of any one of claims 2-3, wherein the spacer sequence is complementary to a target nucleotide sequence in a target bacterium.

5. The bacteriophage of claim 4, wherein the target nucleotide sequence comprises a coding sequence.

6. The bacteriophage of claim 4, wherein the target nucleotide sequence comprises a non-coding or intergenic sequence.

7. The bacteriophage of claim 4, wherein the target nucleotide sequence comprises all or a part of a promoter sequence.

8. The bacteriophage of claim 5, wherein the target nucleotide sequence comprises all or a part of a nucleotide sequence located on a coding strand of a transcribed region of an essential gene.

9. The bacteriophage of claim 8 wherein the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK.

10. The bacteriophage of any one of claims 1-9, wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.

11. The bacteriophage of any one of claims 1-10, wherein the Cascade complex comprises:
(i) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system);
(ii) a Cas6b polypeptide, a Cas8b polypeptide, a Cas7 polypeptide, and a Cas5 polypeptide (Type I-B CRISPR-Cas system);
(iii) a Cas5d polypeptide, a Cas8c polypeptide, and a Cas7 polypeptide (Type I-C CRISPR-Cas system);
(iv) a Cas10d polypeptide, a Csc2 polypeptide, a Csc1 polypeptide, a Cas6d polypeptide (Type I-D CRISPR-Cas system);
(v) a Cse1 polypeptide, a Cse2 polypeptide, a Cas7 polypeptide, a Cas5 polypeptide, and a Cas6e polypeptide (Type I-E CRISPR-Cas system);
(vi) a Csy1 polypeptide, a Csy2 polypeptide, a Csy3 polypeptide, and a Csy4 polypeptide (Type I-F CRISPR-Cas system).

12. The bacteriophage of claim 11, wherein the Cas complex comprises a Cas5d polypeptide, a Cas8c polypeptide, and a Cas7 polypeptide (Type I-C CRISPR-Cas system).

13. The bacteriophage of any one of claims 1-12, wherein the nucleic acid sequence further comprises a promoter sequence.

14. The bacteriophage of any one of claims 4-13, wherein the target bacterium is killed solely by lytic activity of the bacteriophage.

15. The bacteriophage of any one of claims 4-14, wherein the target bacterium is killed solely by activity of the CRISPR-Cas system.

16. The bacteriophage of any one of claims 4-13, wherein the target bacterium is killed by lytic activity of the bacteriophage in combination with activity of the CRISPR-Cas system.

17. The bacteriophage of any one of claims 4-11, wherein the target bacterium is killed by the activity of the CRISPR-Cas system, independently of the lytic activity of the bacteriophage.

18. The bacteriophage of any one of claims 4-13, wherein the activity of the CRISPR-Cas system supplements or enhances the lytic activity of the bacteriophage.

19. The bacteriophage of any one of claims 17-18, wherein the lytic activity of the bacteriophage and the activity of the CRISPR-Cas system are synergistic.

20. The bacteriophage of any one of claims 14-19, wherein the lytic activity of the bacteriophage, the activity of the CRISPR-Cas system, or both, is modulated by a concentration of the bacteriophage

21. The bacteriophage of any one of claims 1-20, wherein the bacteriophage infects multiple bacterial strains.

22. The bacteriophage of any one of claims 4-21, wherein the target bacterium is an Acinetobacter species, an Actinomyces species, Burkholderia cepacia complex, a Campylobacter species, a Candida species, Clostridium difficile, Corynebacterium minutissium, Corynebacterium pseudodiphtherias, Corynebacterium stratium, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter felis, Helicobacter pylori, Clostridium bolteae, and any combination thereof.

23. The bacteriophage of any one of claims 1-22, wherein the bacteriophage is an obligate lytic bacteriophage.

24. The bacteriophage of any one of claims 1-23, wherein the bacteriophage is a temperate bacteriophage that is rendered lytic.

25. The bacteriophage of claim 24, wherein the temperate bacteriophage is rendered lytic by the removal, replacement, or inactivation of a lysogeny gene.

26. The bacteriophage of any one of claims 1-24, wherein the bacteriophage is p1772, p2131, p2132, p2973, p4209, p1106, p1587, p1835, p2037, p2421, p2363, p004k, or PB1.

27. The bacteriophage of any one of claims 1-26, wherein the nucleic acid sequence is inserted into a non-essential bacteriophage gene.

28. A pharmaceutical composition comprising:
(a) the bacteriophage of any one of claims 1-27; and
(b) a pharmaceutically acceptable excipient.

29. The pharmaceutical composition of claim 28, wherein the pharmaceutical composition is in the form of a tablet, a liquid, a syrup, an oral formulation, an intravenous formulation, an intranasal formulation, an ocular formulation, an otic formulation, a subcutaneous formulation, an inhalable respiratory formulation, a suppository, and any combination thereof.

30. A method of killing a target bacterium comprising introducing into the target bacterium a bacteriophage comprising a nucleic acid sequence encoding a Type I CRISPR-Cas system comprising:
(a) a CRISPR array;
(b) a Cascade polypeptide; and
(c) a Cas3 polypeptide.

31. The method of claim 30, wherein the CRISPR array comprises a spacer sequence and at least one repeat sequence.

32. The method of claim 31, wherein the at least one repeat sequence is operably linked to the spacer sequence at either its 5′ end or its 3′ end.

33. The method of any one of claims 30-32 wherein the spacer sequence is complementary to a target nucleotide sequence in a target bacterium.

34. The method of claim 33, wherein the target nucleotide sequence comprises a coding sequence.

35. The method of claim 33, wherein the target nucleotide sequence comprises a non-coding or intergenic sequence.

36. The method of claim 33, wherein the target nucleotide sequence comprises all or a part of a promoter sequence.

37. The method of claim 34, wherein the target nucleotide sequence comprises all or a part of a nucleotide sequence located on a coding strand of a transcribed region of an essential gene.

38. The method of claim 37, wherein the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK.

39. The method of any one of claims 30-38, wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.

40. The method of any one of claims 30-39, wherein the Cascade complex comprises:
(i) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system);
(ii) a Cas6b polypeptide, a Cas8b polypeptide, a Cas7 polypeptide, and a Cas5 polypeptide (Type I-B CRISPR-Cas system);
(iii) a Cas5d polypeptide, a Cas8c polypeptide, and a Cas7 polypeptide (Type I-C CRISPR-Cas system);
(iv) a Cas10d polypeptide, a Csc2 polypeptide, a Csc1 polypeptide, a Cas6d polypeptide (Type I-D CRISPR-Cas system);
(v) a Cse1 polypeptide, a Cse2 polypeptide, a Cas7 polypeptide, a Cas5 polypeptide, and a Cas6e polypeptide (Type I-E CRISPR-Cas system);
(vi) a Csy1 polypeptide, a Csy2 polypeptide, a Csy3 polypeptide, and a Csy4 polypeptide (Type I-F CRISPR-Cas system).

41. The method of claim 40, wherein the Cascade complex comprises a Cas5d polypeptide, a Cas8c polypeptide, and a Cas7 polypeptide (Type I-C CRISPR-Cas system).

42. The method of any one of claims 30-41, wherein the nucleic acid sequence further comprises a promoter sequence.

43. The method of any one of claims 33-41, wherein the target bacterium is killed solely by activity of the CRISPR-Cas system.

44. The method of any one of claims 33-41, wherein the target bacterium is killed by lytic activity of the bacteriophage in combination with activity of the CRISPR-Cas system.

45. The method of any one of claims 33-41, wherein the target bacterium is killed by the activity of the CRISPR-Cas system, independently of the lytic activity of the bacteriophage.

46. The method of any one of claims 33-41, wherein the activity of the CRISPR-Cas system supplements or enhances the lytic activity of the bacteriophage.

47. The method of any one of claims 33-41, wherein the lytic activity of the bacteriophage and the activity of the CRISPR-Cas system are synergistic.

48. The method of any one of claims 42-47, wherein the lytic activity of the bacteriophage, the activity of the CRISPR-Cas system, or both, is modulated by a concentration of the bacteriophage

49. The method of any one of claims 30-48, wherein the bacteriophage infects multiple bacterial strains.

50. The method of any one of claims 33-49, wherein the target bacterium is an Acinetobacter species, an Actinomyces species, Burkholderia cepacia complex, a Campylobacter species, a Candida species, Clostridium difficile, Corynebacterium minutissium, Corynebacterium pseudodiphtheriae, Corynebacterium stratium, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter felis, Helicobacter pylori, Clostridium bolteae, and any combination thereof.

51. The method of any one of claims 30-50, wherein the bacteriophage is an obligate lytic bacteriophage.

52. The method of any one of claims 30-50, wherein the bacteriophage is a temperate bacteriophage that is rendered lytic.

53. The method of claim 52, wherein the temperate bacteriophage is rendered lytic by the removal, replacement, or inactivation of a lysogeny gene.

54. The method of any one of claims 30-53, wherein the bacteriophage is p1772, p2131, p2132, p2973, p4209, p1106, p1587, p1835, p2037, p2421, p2363, p4209, p004k, or PB1.

55. The method of any one of claims 30-54, wherein the nucleic acid sequence is inserted in pace of or adjacent to a non-essential bacteriophage gene.

56. The method of any one of claims 30-55, wherein a mixed population of bacterial cells comprises the target bacterium.

57. A method of treating a disease in an individual in need thereof, the method comprising administering to the individual a bacteriophage comprising a nucleic acid sequence encoding a Type I CRISPR-Cas system comprising:
(a) a CRISPR array;
(b) a Cascade polypeptide; and
(c) a Cas3 polypeptide.

58. The method of claim 57, wherein the CRISPR array comprises a spacer sequence and at least one repeat sequence.

59. The method of claim 58, wherein the at least one repeat sequence is operably linked to the spacer sequence at either its 5′ end or its 3′ end.

60. The method of any one of claims 57-59, wherein the spacer sequence is complementary to a target nucleotide sequence in a target bacterium.

61. The method of claim 60, wherein the target nucleotide sequence comprises a coding sequence.

62. The method of claim 60, wherein the target nucleotide sequence comprises a non-coding or intergenic sequence.

63. The method of claim 60, wherein the target nucleotide sequence comprises all or a part of a promoter sequence.

64. The method of claim 61, wherein the target nucleotide sequence comprises all or a part of a nucleotide sequence located on a coding strand of a transcribed region of an essential gene.

65. The method of claim 64, wherein the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK.

66. The method of any one of claims 57-65, wherein the Cascade complex comprises Cascade polypeptides of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.

67. The method of any one of claims 57-66, wherein the Cascade complex comprises:
(i) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system);
(ii) a Cas6b polypeptide, a Cas8b polypeptide, a Cas7 polypeptide, and a Cas5 polypeptide (Type I-B CRISPR-Cas system);
(iii) a Cas5d polypeptide, a Cas8c polypeptide, and a Cas7 polypeptide (Type I-C CRISPR-Cas system);
(iv) a Cas10d polypeptide, a Csc2 polypeptide, a Csc1 polypeptide, a Cas6d polypeptide (Type I-D CRISPR-Cas system);
(v) a Cse1 polypeptide, a Cse2 polypeptide, a Cas7 polypeptide, a Cas5 polypeptide, and a Cas6e polypeptide (Type I-E CRISPR-Cas system);
(vi) a Csy1 polypeptide, a Csy2 polypeptide, a Csy3 polypeptide, and a Csy4 polypeptide (Type I-F CRISPR-Cas system).

68. The method of claim 67, wherein the CASCADE complex comprises a Cas5d polypeptide, a Cas8c polypeptide, and a Cas7 polypeptide (Type I-C CRISPR-Cas system).

69. The method of any one of claims 57-68, wherein the nucleic acid sequence further comprises a promoter sequence.

70. The method of any one of claims 60-69, wherein the target bacterium is killed solely by activity of the CRISPR-Cas system.

71. The method of any one of claims 60-69, wherein the target bacterium is killed by lytic activity of the bacteriophage in combination with activity of the CRISPR-Cas system.

72. The method of any one of claims 60-69, wherein the target bacterium is killed by the activity of the CRISPR-Cas system, independently of the lytic activity of the bacteriophage.

73. The method of any one of claims 60-69, wherein the activity of the CRISPR-Cas system supplements or enhances the lytic activity of the bacteriophage.

74. The method of any one of claims 60-69, wherein the lytic activity of the bacteriophage and the activity of the CRISPR-Cas system are synergistic.

75. The method of any one of claims 60-74, wherein the lytic activity of the bacteriophage, the activity of the CRISPR-Cas system, or both, is modulated by a concentration of the bacteriophage

76. The method of any one of claims 57-75, wherein the bacteriophage infects multiple bacterial strains.

77. The method of any one of claims 57-76, wherein the bacteriophage is an obligate lytic bacteriophage.

78. The method of any one of claims 57-77, wherein the bacteriophage is a temperate bacteriophage that is rendered lytic.

79. The method of claim 78, wherein the temperate bacteriophage is rendered lytic by the removal, replacement, or inactivation of a lysogeny gene.

80. The method of any one of claims 57-79, wherein the bacteriophage is p1772, p2131, p2132, p2973, p4209, p1106, p1587, p1835, p2037, p2421, p2363, p4209, p004k, or PB1.

81. The method of any one of claims 57-80, wherein the nucleic acid sequence is inserted in pace of or adjacent to a non-essential bacteriophage gene.

82. The method of any one of claims 57-81, wherein the disease is a bacterial infection.

83. The method of any one of claims 60-82, wherein the target bacterium causing the disease is a drug resistant bacterium that is resistant to at least one antibiotic.

84. The method of claim 83, wherein the drug resistant bacterium is resistant to at least one antibiotic.

85. The method of any one of claims 60-84, wherein the target bacterium causing the disease is a multidrug resistant bacterium.

86. The method of claim 85, wherein the multi-drug resistant bacterium is resistant to at least one antibiotic.

87. The method of any one of claims 83-86, wherein the antibiotic comprises a cephalosporin, a fluoroquinolone, a carbapenem, a colistin, an aminoglycoside, vancomycin, streptomycin, or methicillin.

88. The method of any one of claims 60-87, wherein the target bacterium causing the bacterial infection is an Acinetobacter species, an Actinomyces species, Burkholderia cepacia complex, a Campylobacter species, a Candida species, Clostridium difficile, Corynebacterium minutissium, Corynebacterium pseudodiphtheriae, Corynebacterium stratium, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter felis, Helicobacter pylori, Clostridium bolteae, and any combination thereof.

89. The method of claim 88, wherein the target bacterium causing the disease is Pseudomonas.

90. The method of claim 89, wherein the target bacterium causing the disease is P. aeruginosa.

91. The method of any one of claims 57-90, wherein the administering is intra-arterial, intravenous, intraurethral, intramuscular, oral, subcutaneous, inhalation, or any combination thereof.

92. The method of any one of claims 57-91, wherein the individual is a mammal.

93. A bacteriophage comprising a nucleic acid sequence encoding a Type I CRISPR-Cas system comprising:
(a) a CRISPR array;
(b) a Cascade polypeptide comprising Cas5, Cas8c and Cas7; and
(c) a Cas3 polypeptide.

94. The bacteriophage of claim 93, wherein the CRISPR array comprises a spacer sequence and at least one repeat sequence.

95. The bacteriophage of claim 94, wherein the at least one repeat sequence is operably linked to the spacer sequence at either its 5′ end or its 3′ end.

96. The bacteriophage of any one of claims 93-95, wherein the spacer sequence is complementary to a target nucleotide sequence in a target bacterium.

97. The bacteriophage of claim 96, wherein the target nucleotide sequence comprises a coding sequence.

98. The bacteriophage of claim 96, wherein the target nucleotide sequence comprises a non-coding or intergenic sequence.

99. The bacteriophage of claim 96, wherein the target nucleotide sequence comprises all or a part of a promoter sequence.

100. The bacteriophage of claim 97, wherein the target nucleotide sequence comprises all or a part of a nucleotide sequence located on a coding strand of a transcribed region of an essential gene.

101. The bacteriophage of claim 98, wherein the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK.

102. The bacteriophage of any one of claims 93-101, wherein the nucleic acid sequence further comprises a promoter sequence.

103. The bacteriophage of any one of claims 96-102, wherein the target bacterium is killed solely by lytic activity of the bacteriophage.

104. The bacteriophage of any one of claims 96-102, wherein the target bacterium is killed solely by activity of the CRISPR-Cas system.

105. The bacteriophage of any one of claims 96-102, wherein the target bacterium is killed by lytic activity of the bacteriophage in combination with activity of the CRISPR-Cas system.

106. The bacteriophage of any one of claims 96-102, wherein the target bacterium is killed by the activity of the CRISPR-Cas system, independently of the lytic activity of the bacteriophage.

107. The bacteriophage of any one of claims 96-102, wherein the activity of the CRISPR-Cas system supplements or enhances the lytic activity of the bacteriophage.

108. The bacteriophage of any one of claims 105-107, wherein the lytic activity of the bacteriophage and the activity of the CRISPR-Cas system are synergistic.

109. The bacteriophage of any one of claims 103-108, wherein the lytic activity of the bacteriophage, the activity of the CRISPR-Cas system, or both, is modulated by a concentration of the bacteriophage

110. The bacteriophage of any one of claims 93-109, wherein the bacteriophage infects multiple bacterial strains.

111. The bacteriophage of any one of claims 96-110, wherein the target bacterium is an Acinetobacter species, an Actinomyces species, Burkholderia cepacia complex, a Campylobacter species, a Candida species, Clostridium difficile, Corynebacterium minutissium, Corynebacterium pseudodiphtheriae, Corynebacterium stratium, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens, Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter felis, Helicobacter pylori, Clostridium bolteae, and any combination thereof.

112. The bacteriophage of any one of claims 93-111, wherein the bacteriophage is an obligate lytic bacteriophage.

113. The bacteriophage of any one of claims 93-111, wherein the bacteriophage is a temperate bacteriophage that is rendered lytic.

114. The bacteriophage of claim 113, wherein the temperate bacteriophage is rendered lytic by the removal, replacement, or inactivation of a lysogeny gene.

115. The bacteriophage of any one of claims 93-114, wherein the bacteriophage is p1772, p2131, p2132, p2973, p4209, p1106, p1587, p1835, p2037, p2421, p2363, p4209, p004k, or PB1.

116. The bacteriophage of any one of claims 93-115, wherein the nucleic acid sequence is inserted into a non-essential bacteriophage gene.

117. A pharmaceutical composition comprising:
(a) the bacteriophage of any one of claims 93-116; and
(b) a pharmaceutically acceptable excipient.

118. The pharmaceutical composition of claim 117, wherein the pharmaceutical composition is in the form of a tablet, a liquid, a syrup, an oral formulation, an intravenous formulation, an intranasal formulation, an ocular formulation, an otic formulation, a subcutaneous formulation, an inhalable respiratory formulation, a suppository, and any combination thereof.

119. A method of sanitizing a surface in need thereof, the method comprising administering to the surface a bacteriophage comprising a nucleic acid sequence encoding a Type I CRISPR-Cas system comprising:
(d) a CRISPR array;
(e) a Cascade polypeptide; and
(f) a Cas3 polypeptide.

120. The method of claim 119, wherein the surface is a hospital surface, a vehicle surface, an equipment surface, or an industrial surface.

121. A method of preventing contamination in a food product or a nutritional supplement, the method comprising administering to the a food product or the nutritional supplement a bacteriophage comprising a nucleic acid sequence encoding a Type I CRISPR-Cas system comprising:
(g) a CRISPR array;
(h) a Cascade polypeptide; and
(i) a Cas3 polypeptide.

122. The method of claim 121, wherein the food product or nutritional supplement comprises milk, yoghurt, curd, cheese, fermented milks, milk based fermented products, ice-creams, fermented cereal based products, milk based powders, infant formulae or tablets, liquid suspensions, dried oral supplement, wet oral supplement, or dry-tube-feeding.
